SEQUENCE LISTING (156 sequences)

SEQ ID NO: 1 - DNA, 90 bp, synthetic construct
  misc_feature 1..90: note = Description of sequence: template sequence
  source 1..90: mol_type = other DNA
  variation 32..72: note = n is any one of a, c, g, and t
cacctaatacgactcactatagcggatccgannnnnnnnnnnnnnnnnnnnnnnnnnnnnnnnnnnnnnnnctggctcgaacaagcttgc

SEQ ID NO: 2 - DNA, 40 bp, synthetic construct
  misc_feature 1..40: note = Description of sequence: Aptamer candidate: IL10.58
  source 1..40: mol_type = other DNA
catctcttggtcaaggtgaagttgcgctaacttcgccaga

SEQ ID NO: 3 - DNA, 40 bp, synthetic construct
  misc_feature 1..40: note = Description of sequence: Aptamer candidate: IL10.11
  source 1..40: mol_type = other DNA
ggcgtcttgtataataccattcgcattgtgcaaaatggtc

SEQ ID NO: 4 - DNA, 42 bp, synthetic construct
  misc_feature 1..42: note = Description of sequence: Aptamer candidate: IL10.08
  source 1..42: mol_type = other DNA
gatatcctttaccgctgtccacaatgcggtatacgaattagt

SEQ ID NO: 5 - DNA, 41 bp, synthetic construct
  misc_feature 1..41: note = Description of sequence: Aptamer candidate: IL10.49
  source 1..41: mol_type = other DNA
gaagtgggggatcatatcgtgctgcaaacacggtttgattg

SEQ ID NO: 6 - DNA, 42 bp, synthetic construct
  misc_feature 1..42: note = Description of sequence: Aptamer candidate: IL10.31
  source 1..42: mol_type = other DNA
gatagtcgtatcgatctgtagaaactttctgatacgttccgg

SEQ ID NO: 7 - DNA, 42 bp, synthetic construct
  misc_feature 1..42: note = Description of sequence: Aptamer candidate: IL10.60
  source 1..42: mol_type = other DNA
gatagttctctgtgactgtgtgagactcatcgctgagatccg

SEQ ID NO: 8 - DNA, 42 bp, synthetic construct
  misc_feature 1..42: note = Description of sequence: Aptamer candidate: IL10.9
  source 1..42: mol_type = other DNA
ctacacaagtatgagccctaggcacaaacattgatttctgct

SEQ ID NO: 9 - DNA, 40 bp, synthetic construct
  misc_feature 1..40: note = Description of sequence: Aptamer candidate: IL10.21
  source 1..40: mol_type = other DNA
cattaaaggatagagtattgtgttaggccacacactattc

SEQ ID NO: 10 - DNA, 40 bp, synthetic construct
  misc_feature 1..40: note = Description of sequence: Aptamer candidate: IL10.38
  source 1..40: mol_type = other DNA
ccgatcttacacagaagcaggtctctgctctccggcagta

SEQ ID NO: 11 - DNA, 40 bp, synthetic construct
  misc_feature 1..40: note = Description of sequence: Aptamer candidate: IL10.28
  source 1..40: mol_type = other DNA
cgttgggcgtcgctggacagtgttggaccgacggctggac

SEQ ID NO: 12 - DNA, 40 bp, synthetic construct
  misc_feature 1..40: note = Description of sequence: Aptamer candidate: 2xT. IL10.30
  source 1..40: mol_type = other DNA
cccgggctggtgggctaggaagcaattggatccctggcta

SEQ ID NO: 13 - DNA, 40 bp, synthetic construct
  misc_feature 1..40: note = Description of sequence: Aptamer candidate: 2xT.IL10.13
  source 1..40: mol_type = other DNA
tgcaaaatagtgcacgcgggggagttgacgggctgatctg

SEQ ID NO: 14 - DNA, 40 bp, synthetic construct
  misc_feature 1..40: note = Description of sequence: Aptamer candidate: 2xT.IL10.57
  source 1..40: mol_type = other DNA
cctacaacaccacaaggcgttgatgtagtgaggattgtta

SEQ ID NO: 15 - DNA, 40 bp, synthetic construct
  misc_feature 1..40: note = Description of sequence: Aptamer candidate: 2xT.IL10.33
  source 1..40: mol_type = other DNA
tagggtgaccacaggggtgataatctatcgcctcgaaagt

SEQ ID NO: 16 - DNA, 40 bp, synthetic construct
  misc_feature 1..40: note = Description of sequence: Aptamer candidate: 2xT.IL10.39
  source 1..40: mol_type = other DNA
cttgggaccgctgttaagaaaaaagtgtcgcaagcgggaa

SEQ ID NO: 17 - DNA, 40 bp, synthetic construct
  misc_feature 1..40: note = Description of sequence: Aptamer candidate: 2xT.IL10.37
  source 1..40: mol_type = other DNA
aggcgggggctagatccgaagggaaggactgtaagtcccc

SEQ ID NO: 18 - DNA, 40 bp, synthetic construct
  misc_feature 1..40: note = Description of sequence: Aptamer candidate: 2xT.IL10.14
  source 1..40: mol_type = other DNA
cgccctggctatcgttaaagccgctacgatgcagagtgca

SEQ ID NO: 19 - DNA, 40 bp, synthetic construct
  misc_feature 1..40: note = Description of sequence: Aptamer candidate: 2xT.IL10.27
  source 1..40: mol_type = other DNA
aagatgtgccgcaggccgagatcctaacttcagtcactca

SEQ ID NO: 20 - DNA, 40 bp, synthetic construct
  misc_feature 1..40: note = Description of sequence: Aptamer candidate: 2xT.IL 10.4
  source 1..40: mol_type = other DNA
gcactcgagcggtccgggccgcactctcggcattgcgcaa

SEQ ID NO: 21 - DNA, 40 bp, synthetic construct
  misc_feature 1..40: note = Description of sequence: Aptamer candidate: 2xT.IL 10.75
  source 1..40: mol_type = other DNA
atggtgtgttggactacatagacacctcgagggtgagggg

SEQ ID NO: 22 - DNA, 40 bp, synthetic construct
  misc_feature 1..40: note = Description of sequence: Aptamer candidate: 2rn.IL10.2
  source 1..40: mol_type = other DNA
caacccatgtattacgggtaaccggatcagaaacccagtg

SEQ ID NO: 23 - DNA, 40 bp, synthetic construct
  misc_feature 1..40: note = Description of sequence: Aptamer candidate: 2rn.IL10.3
  source 1..40: mol_type = other DNA
tagggccgttgttggtcagggggaaacgcctggacgatct

SEQ ID NO: 24 - DNA, 40 bp, synthetic construct
  misc_feature 1..40: note = Description of sequence: Aptamer candidate: 2rn.IL10.4
  source 1..40: mol_type = other DNA
tgggcataccactaggggacggatcgacaaaagggcgacg

SEQ ID NO: 25 - DNA, 40 bp, synthetic construct
  misc_feature 1..40: note = Description of sequence: Aptamer candidate: 2rn.IL10.10
  source 1..40: mol_type = other DNA
ggtaccatgggtacctcatactcagcattcaagccagaac

SEQ ID NO: 26 - DNA, 40 bp, synthetic construct
  misc_feature 1..40: note = Description of sequence: Aptamer candidate: 2rm.IL10.13
  source 1..40: mol_type = other DNA
atagtggcagggtactcttagagcggatggatggaggagc

SEQ ID NO: 27 - DNA, 40 bp, synthetic construct
  misc_feature 1..40: note = Description of sequence: Aptamer candidate: 2rn.IL10.14
  source 1..40: mol_type = other DNA
cacgcgggaagtcggtggtagtgatctatgggcgcgaccg

SEQ ID NO: 28 - DNA, 40 bp, synthetic construct
  misc_feature 1..40: note = Description of sequence: Aptamer candidate: 2rn.IL10.23
  source 1..40: mol_type = other DNA
atacaactgtcttgtcagggtgttcggggagttctgaggc

SEQ ID NO: 29 - DNA, 40 bp, synthetic construct
  misc_feature 1..40: note = Description of sequence: Aptamer candidate: 2rn.IL10.25
  source 1..40: mol_type = other DNA
ttggttatggccaaagtaacgcctgagcgttggtcggtta

SEQ ID NO: 30 - DNA, 40 bp, synthetic construct
  misc_feature 1..40: note = Description of sequence: Aptamer candidate: 2rn.IL10.22
  source 1..40: mol_type = other DNA
ctatttatgaggaatatcccagacgggacatcagcatgcc

SEQ ID NO: 31 - DNA, 40 bp, synthetic construct
  misc_feature 1..40: note = Description of sequence: Aptamer candidate: 2rn.IL10.5
  source 1..40: mol_type = other DNA
gctcgcagtgtatacggtctgctaacagctcagagagaca

SEQ ID NO: 32 - DNA, 40 bp, synthetic construct
  misc_feature 1..40: note = Description of sequence: Aptamer candidate: IL12.36
  source 1..40: mol_type = other DNA
tgcaacggggccgacccgacattttggcagtgggagaacg

SEQ ID NO: 33 - DNA, 40 bp, synthetic construct
  misc_feature 1..40: note = Description of sequence: Aptamer candidate: IL12.35
  source 1..40: mol_type = other DNA
ggcctagggacacgctctccaacaagagcatgaggggcaa

SEQ ID NO: 34 - DNA, 40 bp, synthetic construct
  misc_feature 1..40: note = Description of sequence: Aptamer candidate: IL12.13
  source 1..40: mol_type = other DNA
aggtaacagttaccaatctttttgaggatagcatttggaa

SEQ ID NO: 35 - DNA, 40 bp, synthetic construct
  misc_feature 1..40: note = Description of sequence: Aptamer candidate: IL12.28
  source 1..40: mol_type = other DNA
ggcatttgggatttggttatgttaacaaggcatgggcggg

SEQ ID NO: 36 - DNA, 40 bp, synthetic construct
  misc_feature 1..40: note = Description of sequence: Aptamer candidate: Il12.19
  source 1..40: mol_type = other DNA
gacaagtaatcatcgagattcggatttacggtggtgtgca

SEQ ID NO: 37 - DNA, 40 bp, synthetic construct
  misc_feature 1..40: note = Description of sequence: Aptamer candidate: IL12.20
  source 1..40: mol_type = other DNA
agttggcctaatgtaacgccaaggtgccaagtgtttctat

SEQ ID NO: 38 - DNA, 40 bp, synthetic construct
  misc_feature 1..40: note = Description of sequence: Aptamer candidate: IL12.10
  source 1..40: mol_type = other DNA
tcggactggtttgagggttgcggaacagagccgtacccca

SEQ ID NO: 39 - DNA, 40 bp, synthetic construct
  misc_feature 1..40: note = Description of sequence: Aptamer candidate: IL12.33
  source 1..40: mol_type = other DNA
tgtgatgtatattaagcgtgggcctctatcgcacgcttac

SEQ ID NO: 40 - DNA, 40 bp, synthetic construct
  misc_feature 1..40: note = Description of sequence: Aptamer candidate: IL12.5
  source 1..40: mol_type = other DNA
acacgacatatccacgttaaccacataatccgctgtggta

SEQ ID NO: 41 - DNA, 40 bp, synthetic construct
  misc_feature 1..40: note = Description of sequence: Aptamer candidate: IL12.4
  source 1..40: mol_type = other DNA
caaattggcacctactcgtagaataaggtaattgtgctct

SEQ ID NO: 42 - DNA, 40 bp, synthetic construct
  misc_feature 1..40: note = Description of sequence: Aptamer candidate: IL12.3
  source 1..40: mol_type = other DNA
tacacttgtacacgtaatcgtagggggacggtaacgggtc

SEQ ID NO: 43 - DNA, 41 bp, synthetic construct
  misc_feature 1..41: note = Description of sequence: Aptamer candidate: T.7
  source 1..41: mol_type = other DNA
ctaaaagaataggtgatggggatatgggatcataggtgcac

SEQ ID NO: 44 - DNA, 40 bp, synthetic construct
  misc_feature 1..40: note = Description of sequence: Aptamer candidate: T.10
  source 1..40: mol_type = other DNA
ggggatacccctctacagcaattgcgattacaccccttgt

SEQ ID NO: 45 - DNA, 40 bp, synthetic construct
  misc_feature 1..40: note = Description of sequence: Aptamer candidate: T.57
  source 1..40: mol_type = other DNA
gctcggtgtttatatggagtgacgccaccggggacgggag

SEQ ID NO: 46 - DNA, 40 bp, synthetic construct
  misc_feature 1..40: note = Description of sequence: Aptamer candidate: T.69
  source 1..40: mol_type = other DNA
ctggcggggcactcgggagaaaacggagagcacttaactg

SEQ ID NO: 47 - DNA, 40 bp, synthetic construct
  misc_feature 1..40: note = Description of sequence: Aptamer candidate: T.1
  source 1..40: mol_type = other DNA
gttataagtgacgtaccctcagagcatatgcttggtgtgg

SEQ ID NO: 48 - DNA, 38 bp, synthetic construct
  misc_feature 1..38: note = Description of sequence: Aptamer candidate: T.2
  source 1..38: mol_type = other DNA
ttagcactttatttaggttttcgggccacgtgtggttg

SEQ ID NO: 49 - DNA, 40 bp, synthetic construct
  misc_feature 1..40: note = Description of sequence: Aptamer candidate: T.3
  source 1..40: mol_type = other DNA
cggcatattggtgtgttgaatcgcttagattaaggaacct

SEQ ID NO: 50 - DNA, 39 bp, synthetic construct
  misc_feature 1..39: note = Description of sequence: Aptamer candidate: T.4
  source 1..39: mol_type = other DNA
gtgcttcatttaccttcaggttggggattgtgatagagc

SEQ ID NO: 51 - DNA, 40 bp, synthetic construct
  misc_feature 1..40: note = Description of sequence: Aptamer candidate: T.23
  source 1..40: mol_type = other DNA
cagcagcacagtgatgtttgccgaccagggttatgaggtt

SEQ ID NO: 52 - DNA, 90 bp, synthetic construct; identical to SEQ ID NO: 1
  misc_feature 1..90: note = Description of sequence: Name: Aptamer Library (N40)
  source 1..90: mol_type = other DNA
  variation 31..72: note = n is any one of a, c, g, and t

SEQ ID NO: 53 - DNA, 26 bp, synthetic construct
  misc_feature 1..26: note = Description of sequence: Name: Forward primer
  source 1..26: mol_type = other DNA
cacctaatacgactcactatagcgga

SEQ ID NO: 54 - DNA, 19 bp, synthetic construct
  misc_feature 1..19: note = Description of sequence: Name: Reverse primer
  source 1..19: mol_type = other DNA
gcaagcttgttcgagccag

SEQ ID NO: 55 - DNA, 19 bp, synthetic construct
  misc_feature 1..19: note = Description of sequence: Name: Reverse biotin primer
  source 1..19: mol_type = other DNA
  modified_base 1: mod_base = OTHER; note = biotinylated
gcaagcttgttcgagccag

SEQ ID NO: 56 - DNA, 56 bp, synthetic construct
  misc_feature 1..56: note = Description of sequence: Name: Extended forward primer
  source 1..56: mol_type = other DNA
aaaaaaaaaaaaaaaaaaaaaaaaaaaaaacacctaatacgactcactatagcgga

SEQ ID NO: 57 - DNA, 49 bp, synthetic construct
  misc_feature 1..49: note = Description of sequence: Name: Extended reverse primer
  source 1..49: mol_type = other DNA
aaaaaaaaaaaaaaaaaaaaaaaaaaaaaagcaagcttgttcgagccag

SEQ ID NO: 58 - DNA, 18 bp, synthetic construct
  misc_feature 1..18: note = Description of sequence: Name: Thrombin AS-18
  source 1..18: mol_type = other DNA
gatatgggatcataggtg

SEQ ID NO: 59 - DNA, 21 bp, synthetic construct
  misc_feature 1..21: note = Description of sequence: Name: Thrombin AS-21
  source 1..21: mol_type = other DNA
ggatcataggtgcacctggct

SEQ ID NO: 60 - DNA, 24 bp, synthetic construct
  misc_feature 1..24: note = Description of sequence: Name: Thrombin AS-24
  source 1..24: mol_type = other DNA
ggatcataggtgcacctggctcga

SEQ ID NO: 61-66 - skipped (6 entries)

SEQ ID NO: 67 - DNA, 29 bp, synthetic construct
  misc_feature 1..29: note = Description of sequence: Name: T29 Scrambled
  source 1..29: mol_type = other DNA
ctcttggccaattggccaatcatgcatta

SEQ ID NO: 68 - DNA, 90 bp, synthetic construct
  misc_feature 1..90: note = Description of sequence: Name: T-7 (90)
  source 1..90: mol_type = other DNA
cacctaatacgactcactatagcggatccgactaaagaataggtgatggggatatgggatcataggtgcacctggctcgaacaagcttgc

SEQ ID NO: 69 - DNA, 59 bp, synthetic construct
  misc_feature 1..59: note = Description of sequence: Name: T-7 (59)
  source 1..59: mol_type = other DNA
ctaaagaataggtgatggggatatgggatcataggtgcacctggctcgaacaagcttgc

SEQ ID NO: 70 - DNA, 71 bp, synthetic construct
  misc_feature 1..71: note = Description of sequence: Name: T-7 (71)
  source 1..71: mol_type = other DNA
cacctaatacgactcactatagcggatccgactaaagaataggtgatggggatatgggatcataggtgcac

SEQ ID NO: 71 - DNA, 40 bp, synthetic construct
  misc_feature 1..40: note = Description of sequence: Name: T-7 (40)
  source 1..40: mol_type = other DNA
ctaaagaataggtgatggggatatgggatcataggtgcac

SEQ ID NO: 72 - DNA, 46 bp, synthetic construct
  misc_feature 1..46: note = Description of sequence: Name: T-7 (46)
  source 1..46: mol_type = other DNA
gatggggatatgggatcataggtgcacctggctcgaacaagcttgc

SEQ ID NO: 73 - DNA, 30 bp, synthetic construct
  misc_feature 1..30: note = Description of sequence: Name: T-7 (30)
  source 1..30: mol_type = other DNA
gatggggatatgggatcataggtgcacctg

SEQ ID NO: 74 - DNA, 44 bp, synthetic construct
  misc_feature 1..44: note = Description of sequence: Name: T-7 (45.1)
  source 1..44: mol_type = other DNA
cacctaatacgctcactatagcggatccgactaaagaataggtg

SEQ ID NO: 75 - DNA, 40 bp, synthetic construct
  misc_feature 1..40: note = Description of sequence: Name: T-7 (45.2)
  source 1..40: mol_type = other DNA
gatatgggatcataggtgcacctggctcgaacaagcttgc

SEQ ID NO: 76 - DNA, 91 bp, synthetic construct
  misc_feature 1..91: note = Description of sequence: candidate sequence
  source 1..91: mol_type = other DNA
cacctaatacgactcactatagcggatccgagaagtgggggatcatatcgtgctgcaaacacggtttgattgctggctcgaacaagcttgc

SEQ ID NO: 77 - DNA, 90 bp, synthetic construct
  misc_feature 1..90: note = Description of sequence: candidate sequence
  source 1..90: mol_type = other DNA
cacctaatacgactcactatagcggatccgatagggccgttgttggtcagggggaaacgcctggacgatctctggctcgaacaagcttgc

SEQ ID NO: 78 - DNA, 90 bp, synthetic construct
  misc_feature 1..90: note = Description of sequence: candidate sequence
  source 1..90: mol_type = other DNA
cacctaatacgactcactatagcggatccgatgcaaaatagtgcacgcgggggagttgacgggctgatctgctggctcgaacaagcttgc

SEQ ID NO: 79 - DNA, 90 bp, synthetic construct
  misc_feature 1..90: note = Description of sequence: candidate sequence
  source 1..90: mol_type = other DNA
cacctaatacgactcactatagcggatccgaggcctagggacacgctctccaacaagagcatgaggggcaactggctcgaacaagcttgc

SEQ ID NO: 80 - DNA, 76 bp, synthetic construct
  misc_feature 1..76: note = Description of sequence: candidate sequence
  source 1..76: mol_type = other DNA
cacctaatacgactcactatagcggatccgaaaaatgtacggggggaagagactcttctggctcgaacaagcttgc

SEQ ID NO: 81 - DNA, 89 bp, synthetic construct
  misc_feature 1..89: note = Description of sequence: candidate sequence
  source 1..89: mol_type = other DNA
cacctaatacgactcactatagcggatccgaggcctagggacacgcttccaacaagagcatgaggggcaactggctcgaacaagcttgc

SEQ ID NO: 82 - DNA, 90 bp, synthetic construct
  misc_feature 1..90: note = Description of sequence: candidate sequence
  source 1..90: mol_type = other DNA
cacctaatacgactcactatagcggatccgaaaggcgccgagacgtagtaataagtcgggggaaaggtgaactggctcgaacaagcttgc

SEQ ID NO: 83 - DNA, 77 bp, synthetic construct
  misc_feature 1..77: note = Description of sequence: Name: SscambledKK
  source 1..77: mol_type = other DNA
ggagagagagagaatataagaaaacagcagatcagtctatcttctcctgatgggttcctatttataggtgaagctgt

SEQ ID NO: 84 - DNA, 45 bp, synthetic construct
  misc_feature 1..45: note = Description of sequence: Name: T-7 (45)
  source 1..45: mol_type = other DNA
cacctaatacgactcactatagcggatccgactaaagaataggtg

SEQ ID NO: 85 - DNA, 42 bp, synthetic construct; identical to SEQ ID NO: 4
  misc_feature 1..42: note = Description of sequence: Aptamer candidate: IL 10.8
  source 1..42: mol_type = other DNA

SEQ ID NO: 86 - DNA, 40 bp, synthetic construct
  misc_feature 1..40: note = Description of sequence: Aptamer candidate: GM.1
  source 1..40: mol_type = other DNA
ttccaaagggcagtcaagtgtttctatctcgtccggtata

SEQ ID NO: 87 - DNA, 40 bp, synthetic construct
  misc_feature 1..40: note = Description of sequence: Aptamer candidate: GM.6
  source 1..40: mol_type = other DNA
tacatcaataaatgtttgcatattgatctgtatgtttaga

SEQ ID NO: 88 - DNA, 40 bp, synthetic construct
  misc_feature 1..40: note = Description of sequence: Aptamer candidate: GM.14
  source 1..40: mol_type = other DNA
aaaattgtgctggttcaatgtacggggggaagagactctt

SEQ ID NO: 89 - DNA, 40 bp, synthetic construct
  misc_feature 1..40: note = Description of sequence: Aptamer candidate: GM.17
  source 1..40: mol_type = other DNA
ttaatttcaggtttagcagtactttgaaatttttaagttt

SEQ ID NO: 90 - DNA, 40 bp, synthetic construct
  misc_feature 1..40: note = Description of sequence: Aptamer candidate: GM.23
  source 1..40: mol_type = other DNA
gacagtccagtacggaaataatattcgaaccatttgttat

SEQ ID NO: 91 - DNA, 40 bp, synthetic construct
  misc_feature 1..40: note = Description of sequence: Aptamer candidate: GM.31
  source 1..40: mol_type = other DNA
attaatagaatgagatgtatctgggtaagtttttaagtat

SEQ ID NO: 92 - DNA, 40 bp, synthetic construct
  misc_feature 1..40: note = Description of sequence: Aptamer candidate: GM.32
  source 1..40: mol_type = other DNA
atctataacgagaaatgttaagtcttttctaatcacctga

SEQ ID NO: 93 - DNA, 40 bp, synthetic construct
  misc_feature 1..40: note = Description of sequence: Aptamer candidate: GM.33
  source 1..40: mol_type = other DNA
ttgtggaaaaatcatttcagataatttattaatgttgcat

SEQ ID NO: 94 - DNA, 40 bp, synthetic construct
  misc_feature 1..40: note = Description of sequence: Aptamer candidate: GM.41
  source 1..40: mol_type = other DNA
ccgaagtatgttttcaattgcacctagttttttctatctt

SEQ ID NO: 95 - DNA, 40 bp, synthetic construct
  misc_feature 1..40: note = Description of sequence: Aptamer candidate: GM.46
  source 1..40: mol_type = other DNA
tctattttctagcaacttttatatgagttctttctgttgg

SEQ ID NO: 96 - DNA, 40 bp, synthetic construct
  misc_feature 1..40: note = Description of sequence: Aptamer candidate: GM.57
  source 1..40: mol_type = other DNA
tgttggttattttacgtcacagaccgactagacattgtct

SEQ ID NO: 97 - DNA, 40 bp, synthetic construct
  misc_feature 1..40: note = Description of sequence: Aptamer candidate: BDNF.3
  source 1..40: mol_type = other DNA
cttatagcttgggaaaagtccctaatcagatgatgtttca

SEQ ID NO: 98 - DNA, 40 bp, synthetic construct
  misc_feature 1..40: note = Description of sequence: Aptamer candidate: BDNF.7
  source 1..40: mol_type = other DNA
atgcgccaatttaggtataaaatgtaaagagtcatgatag

SEQ ID NO: 99 - DNA, 41 bp, synthetic construct
  misc_feature 1..41: note = Description of sequence: Aptamer candidate: BDNF.8
  source 1..41: mol_type = other DNA
tgttcaataaagaatgttattgtattaacagatagtttcaa

SEQ ID NO: 100 - DNA, 40 bp, synthetic construct
  misc_feature 1..40: note = Description of sequence: Aptamer candidate: BDNF.11
  source 1..40: mol_type = other DNA
gtagttagatttcatctttgcttgtatgcttatgttataa

SEQ ID NO: 101 - DNA, 40 bp, synthetic construct
  misc_feature 1..40: note = Description of sequence: Aptamer candidate: BDNF.12
  source 1..40: mol_type = other DNA
gactgatgttacgctgtgcgatcatgcggacgcgcggttg

SEQ ID NO: 102 - DNA, 40 bp, synthetic construct
  misc_feature 1..40: note = Description of sequence: Aptamer candidate: BDNF.14
  source 1..40: mol_type = other DNA
gtcgtgttggtatttggtttgtattgtgaatgcatttata

SEQ ID NO: 103 - DNA, 40 bp, synthetic construct
  misc_feature 1..40: note = Description of sequence: Aptamer candidate: BDNF.34
  source 1..40: mol_type = other DNA
ggagtgtatctatagttcacttgttaggacattagtggcg

SEQ ID NO: 104 - DNA, 40 bp, synthetic construct
  misc_feature 1..40: note = Description of sequence: Aptamer candidate: BDNF.46
  source 1..40: mol_type = other DNA
gtgatgaaagatgagttaagtatttgaacaaggtctatac

SEQ ID NO: 105 - DNA, 40 bp, synthetic construct
  misc_feature 1..40: note = Description of sequence: Aptamer candidate: BDNF.48
  source 1..40: mol_type = other DNA
aaggcgccgagacgtagtaataagtcgggggaaaggtgaa

SEQ ID NO: 106 - DNA, 40 bp, synthetic construct
  misc_feature 1..40: note = Description of sequence: Aptamer candidate: BDNF.53
  source 1..40: mol_type = other DNA
accattatattcgaatggactcaaccagctccaacacata

SEQ ID NO: 107 - DNA, 40 bp, synthetic construct
  misc_feature 1..40: note = Description of sequence: Selected Candidate
  source 1..40: mol_type = other DNA
ttggtcgaatagctcgaaggtccatacaggggtgtaagct

SEQ ID NO: 108 - DNA, 40 bp, synthetic construct
  misc_feature 1..40: note = Description of sequence: Selected Candidate
  source 1..40: mol_type = other DNA
tgtaggaggggaccgggctagttatatttcacctctatcg

SEQ ID NO: 109 - DNA, 40 bp, synthetic construct
  misc_feature 1..40: note = Description of sequence: Selected Candidate
  source 1..40: mol_type = other DNA
gctcttaatgttaaggggtacagtccgaacaattctggtg

SEQ ID NO: 110 - DNA, 40 bp, synthetic construct
  misc_feature 1..40: note = Description of sequence: Selected Candidate
  source 1..40: mol_type = other DNA
gcagtaggggcgtgtccaagtctactgagccgacagatgg

SEQ ID NO: 111 - DNA, 40 bp, synthetic construct
  misc_feature 1..40: note = Description of sequence: Selected Candidate
  source 1..40: mol_type = other DNA
atagggggcttccacgtcctgatagaatagaataatgagt

SEQ ID NO: 112 - DNA, 40 bp, synthetic construct
  misc_feature 1..40: note = Description of sequence: Selected Candidate
  source 1..40: mol_type = other DNA
cgtcatacccaaatataagtataccagagaggggccatta

SEQ ID NO: 113 - DNA, 40 bp, synthetic construct; identical to SEQ ID NO: 71
  misc_feature 1..40: note = Description of sequence: Selected Candidate
  source 1..40: mol_type = other DNA

SEQ ID NO: 114 - DNA, 40 bp, synthetic construct
  misc_feature 1..40: note = Description of sequence: Selected Candidate
  source 1..40: mol_type = other DNA
gatctggggtcgttattttacggtagaacgcattttggta

SEQ ID NO: 115 - DNA, 40 bp, synthetic construct
  misc_feature 1..40: note = Description of sequence: Selected Candidate
  source 1..40: mol_type = other DNA
acgccccgaatgtaggtccgtcgtggtctggggttgttta

SEQ ID NO: 116 - DNA, 40 bp, synthetic construct; identical to SEQ ID NO: 44
  misc_feature 1..40: note = Description of sequence: Selected Candidate
  source 1..40: mol_type = other DNA

SEQ ID NO: 117 - DNA, 40 bp, synthetic construct
  misc_feature 1..40: note = Description of sequence: Selected Candidate
  source 1..40: mol_type = other DNA
ccttaagccaccgggggcatgacctgtttctttattattc

SEQ ID NO: 118 - DNA, 39 bp, synthetic construct
  misc_feature 1..39: note = Description of sequence: Selected Candidate
  source 1..39: mol_type = other DNA
ttcttttgaatgtgctggggttactcgagtggtatctat

SEQ ID NO: 119 - DNA, 40 bp, synthetic construct
  misc_feature 1..40: note = Description of sequence: Selected Candidate
  source 1..40: mol_type = other DNA
ctcggtatttatccatacttggggtacgatcgttttagtc

SEQ ID NO: 120 - DNA, 41 bp, synthetic construct
  misc_feature 1..41: note = Description of sequence: Selected Candidate
  source 1..41: mol_type = other DNA
ggattccggggctagtgtaggtttcagtttcgttacatgac

SEQ ID NO: 121 - DNA, 40 bp, synthetic construct
  misc_feature 1..40: note = Description of sequence: Selected Candidate
  source 1..40: mol_type = other DNA
tggggagtaagctgaggctgaacctatcttacctgagtcc

SEQ ID NO: 122 - DNA, 40 bp, synthetic construct
  misc_feature 1..40: note = Description of sequence: Selected Candidate
  source 1..40: mol_type = other DNA
ggggattatggtaaatggcagtacacttcccaggttttca

SEQ ID NO: 123 - DNA, 40 bp, synthetic construct
  misc_feature 1..40: note = Description of sequence: Selected Candidate
  source 1..40: mol_type = other DNA
gttctactcgacggtatatagttcgtggggatggtcgaca

SEQ ID NO: 124 - DNA, 40 bp, synthetic construct
  misc_feature 1..40: note = Description of sequence: Selected Candidate
  source 1..40: mol_type = other DNA
aaataaccgcccccgagtgggccgtattacaggggtaagt

SEQ ID NO: 125 - DNA, 40 bp, synthetic construct
  misc_feature 1..40: note = Description of sequence: Selected Candidate
  source 1..40: mol_type = other DNA
agtttcattgcatcgtccgggggtcgataccactcacaag

SEQ ID NO: 126 - DNA, 40 bp, synthetic construct
  misc_feature 1..40: note = Description of sequence: Selected Candidate
  source 1..40: mol_type = other DNA
cagagaccatggccctttatgcggatctgttgcggggatc

SEQ ID NO: 127 - DNA, 40 bp, synthetic construct
  misc_feature 1..40: note = Description of sequence: Selected Candidate
  source 1..40: mol_type = other DNA
tttagtagcatcacatcgttttccggggatgggcactaca

SEQ ID NO: 128 - DNA, 39 bp, synthetic construct
  misc_feature 1..39: note = Description of sequence: Selected Candidate
  source 1..39: mol_type = other DNA
gttatcgcggggaatacagggtgaagctagcccgccgga

SEQ ID NO: 129 - DNA, 40 bp, synthetic construct
  misc_feature 1..40: note = Description of sequence: Selected Candidate
  source 1..40: mol_type = other DNA
gccattgggattaaatgcgtggggatccgttttgattgag

SEQ ID NO: 130 - DNA, 40 bp, synthetic construct
  misc_feature 1..40: note = Description of sequence: Selected Candidate
  source 1..40: mol_type = other DNA
acttgaattataacggggtcctgttgagcagattcaacgt

SEQ ID NO: 131 - DNA, 40 bp, synthetic construct
  misc_feature 1..40: note = Description of sequence: Selected Candidate
  source 1..40: mol_type = other DNA
tgtaaacggggatgtctcttcagatgcaggaaatggggac

SEQ ID NO: 132 - DNA, 40 bp, synthetic construct
  misc_feature 1..40: note = Description of sequence: Selected Candidate
  source 1..40: mol_type = other DNA
aatgtacctcggggtaaagttaattagtgggttgtgtgtg

SEQ ID NO: 133 - DNA, 39 bp, synthetic construct
  misc_feature 1..39: note = Description of sequence: Selected Candidate
  source 1..39: mol_type = other DNA
gtccgtacttagcatagttggggcccccgttcatatggg

SEQ ID NO: 134 - DNA, 40 bp, synthetic construct
  misc_feature 1..40: note = Description of sequence: Selected Candidate
  source 1..40: mol_type = other DNA
ttttggtggggagagtgtattgtcctgagtccttgcagct

SEQ ID NO: 135 - DNA, 40 bp, synthetic construct
  misc_feature 1..40: note = Description of sequence: Selected Candidate
  source 1..40: mol_type = other DNA
tcccagttattcaagctcgttctattaacgtattaggggg

SEQ ID NO: 136 - DNA, 40 bp, synthetic construct
  misc_feature 1..40: note = Description of sequence: Selected Candidate
  source 1..40: mol_type = other DNA
aatatgcgcatggagatcactgcgtgcccgcgggggatcg

SEQ ID NO: 137 - DNA, 40 bp, synthetic construct
  misc_feature 1..40: note = Description of sequence: Selected Candidate
  source 1..40: mol_type = other DNA
tagtggggaatgttaatggagtcagaatgtagtctatgag

SEQ ID NO: 138 - DNA, 39 bp, synthetic construct
  misc_feature 1..39: note = Description of sequence: Selected Candidate
  source 1..39: mol_type = other DNA
ttgtccggaccgttcccttgacggggttactttagcaat

SEQ ID NO: 139 - DNA, 40 bp, synthetic construct
  misc_feature 1..40: note = Description of sequence: Selected Candidate
  source 1..40: mol_type = other DNA
gcggggtgaataaacaggtgttatctttgaactgtagggt

SEQ ID NO: 140 - DNA, 40 bp, synthetic construct
  misc_feature 1..40: note = Description of sequence: Selected Candidate
  source 1..40: mol_type = other DNA
gggatttaggaacaaatctgatatggggcctcatacggtt

SEQ ID NO: 141 - DNA, 40 bp, synthetic construct
  misc_feature 1..40: note = Description of sequence: Selected Candidate
  source 1..40: mol_type = other DNA
gctgaagagtaagccgctccgaaggatttaacggggtgaa

SEQ ID NO: 142 - DNA, 40 bp, synthetic construct
  misc_feature 1..40: note = Description of sequence: Selected Candidate
  source 1..40: mol_type = other DNA
gggtgctataccggccggactgtggggttcacttcatggt

SEQ ID NO: 143 - DNA, 40 bp, synthetic construct
  misc_feature 1..40: note = Description of sequence: Selected Candidate
  source 1..40: mol_type = other DNA
ttgttgcattgccttactaattaattggccgagggggagc

SEQ ID NO: 144 - DNA, 40 bp, synthetic construct
  misc_feature 1..40: note = Description of sequence: Selected Candidate
  source 1..40: mol_type = other DNA
ggagtgtagggatgtataggggcgtctgggccacgtgatt

SEQ ID NO: 145 - DNA, 40 bp, synthetic construct
  misc_feature 1..40: note = Description of sequence: Selected Candidate
  source 1..40: mol_type = other DNA
aattcattgatagggggggtgcttcactgagggagatgtt

SEQ ID NO: 146 - DNA, 40 bp, synthetic construct
  misc_feature 1..40: note = Description of sequence: Selected Candidate
  source 1..40: mol_type = other DNA
ctagacattataatagggacttcatgtgagttaaggatgg

SEQ ID NO: 147 - DNA, 40 bp, synthetic construct
  misc_feature 1..40: note = Description of sequence: Selected Candidate
  source 1..40: mol_type = other DNA
acgtcagacgtgtaactcactgtatgtaaatgcggaagtc

SEQ ID NO: 148 - DNA, 40 bp, synthetic construct
  misc_feature 1..40: note = Description of sequence: Selected Candidate
  source 1..40: mol_type = other DNA
aagttgaatttgtaactccacggaattatcattgttccgg

SEQ ID NO: 149 - DNA, 40 bp, synthetic construct
  misc_feature 1..40: note = Description of sequence: Selected Candidate
  source 1..40: mol_type = other DNA
taaaaattaccatgcccgttcaattcgggcaagggacgtg

SEQ ID NO: 150 - DNA, 40 bp, synthetic construct
  misc_feature 1..40: note = Description of sequence: Selected Candidate
  source 1..40: mol_type = other DNA
taaaggcaccccacctaataaaattgccccttagtactat

SEQ ID NO: 151 - DNA, 40 bp, synthetic construct; identical to SEQ ID NO: 146
  misc_feature 1..40: note = Description of sequence: Selected Candidate
  source 1..40: mol_type = other DNA

SEQ ID NO: 152 - DNA, 40 bp, synthetic construct
  misc_feature 1..40: note = Description of sequence: Selected Candidate
  source 1..40: mol_type = other DNA
agaattatatataaatgtaaatgtacggtactacgtctga

SEQ ID NO: 153 - DNA, 40 bp, synthetic construct; identical to SEQ ID NO: 45
  misc_feature 1..40: note = Description of sequence: Selected Candidate
  source 1..40: mol_type = other DNA

SEQ ID NO: 154 - DNA, 40 bp, synthetic construct
  misc_feature 1..40: note = Description of sequence: Selected Candidate
  source 1..40: mol_type = other DNA
taagtaaacatgggagtttgtagctcgagagactctataa

SEQ ID NO: 155 - DNA, 40 bp, synthetic construct
  misc_feature 1..40: note = Description of sequence: Selected Candidate
  source 1..40: mol_type = other DNA
ttacttagatcatggtgatcgatgcgtctcttcgaacatg

SEQ ID NO: 156 - DNA, 40 bp, synthetic construct; identical to SEQ ID NO: 46
  misc_feature 1..40: note = Description of sequence: Selected Candidate
  source 1..40: mol_type = other DNA

(19) United States
(12) Patent Application Publication
(10) Pub. No.: US 2026/0308599 A1
(43) Pub. Date: Oct. 8, 2026

(54) MATERIALS AND METHODS FOR APTAMER SELECTION

(71) Applicant: THE PENN STATE RESEARCH FOUNDATION, University Park, PA (US)

(72) Inventors: Yong WANG, University Park, PA (US); Naveen SINGH, University Park, PA (US)

(21) Appl. No.: 19/133,833

(22) PCT Filed: Nov. 29, 2023
(86) PCT No.: PCT/US2023/081604

§ 371 Date: May 29, 2025

Related U.S. Application Data
(60) Provisional application No. 63/428,466, filed on Nov. 29, 2022.
(60) Provisional application No. 63/472,942, filed on Jun. 14, 2023.

(51) Int. Cl. C12N 15/115

(52) U.S. Cl. CPC C12N 15/115; C12N 2310/16; C12N 2320/10

(57) ABSTRACT

Described are methods for identifying or producing an aptamer to a target. The target can be conjugated to a microporous hydrogel. Described are also aptamer-target complexes including an aptamer and a target, where the target is conjugated to a porous hydrogel, and the aptamer selectively associates with the target.

### CROSS-REFERENCE TO RELATED APPLICATION

This application claims priority to, and the benefit of U.S. Provisional Application 63/428,466, filed on Nov. 29, 2022 and U.S. Provisional Application 63/472,942, filed on Jun. 14, 2023, the contents of which are hereby incorporated in their entirety.

### STATEMENT REGARDING FEDERALLY SPONSORED RESEARCH OR DEVELOPMENT

This invention was made with government support under Grant Nos. HL122311 and AR073364 awarded by National Institutes of Health. The government has certain rights in the invention.

### REFERENCE TO SEQUENCE LISTING

The Sequence Listing submitted Jun. 3, 2026, as a text filed named “11196-092US1_ST26.xml” created Jun. 3, 2026, and having a file size of 201,252 bytes is hereby incorporated by reference pursuant to 37 C.F.R. § 1.52 (e) (5).

### BACKGROUND

Nucleic acid aptamers (or aptamers) are single-stranded oligonucleotides screened from DNA/RNA libraries with high affinities and specificities against target molecules. They are known as “chemical antibodies”. Simply speaking, aptamers can be used in many, if not all, applications where antibodies have been used. These areas include but are not limited to cancer therapy, regenerative medicine, molecular biosensing, cell surface engineering, drug or nanoparticle delivery, detoxification, anti-inflammation, and anti-coagulation. Aptamers possess many desirable characteristics, including high binding affinity, high binding specificity, tolerance of harsh conditions, low immunogenicity or toxicity, low batch-to-batch variation, and/or ease of synthesis.

Aptamers can possess many advantages over antibodies. First, aptamers can be selected in vitro. This is in contrast with antibodies, whose discovery and production relies on the use of live cells or animals. Second, in principle, aptamers can be rigorously selected against any molecules with binding affinities similar to or even superior to antibodies. Aptamers also have remarkable specificity with the ability to discriminate targets based on subtle structural differences. Third, aptamers can be chemically modified against nuclease degradation in biological fluids. Studies have shown that aptamers entirely composed of 2′-O-methyl nucleotides were completely resistant to biodegradation during a 96-h incubation in plasma at 37° C. Moreover, aptamer have a high tolerance to harsh thermal, physical, and chemical conditions. By contrast, antibodies can easily lose bioactivity under harsh conditions. Fourth, aptamers have little immunogenicity or toxicity in comparison to many ligands or biomolecules including antibodies because they are not only composed of nucleotides but also small in size (usually about 20 to 30 nucleotides long). Fifth, aptamers are synthesized with a standard chemical procedure without batch-to-batch variation. Antibody production has to be accomplished in cells or live animals. Therefore, aptamers have been widely studied as “chemical antibodies” for various applications.

The concept of aptamer selection was initially developed in 1990. The selection involves incubation, partition, and amplification in each cycle. The theoretical calculation suggests that one cycle of selection can enrich the library by one to two orders of magnitude even if the selection runs smoothly. Because the initial library contains ~10¹⁴ molecules to ensure rigorous selection, multiple cycles of selection are required to reduce the number of aptamer candidates. It generally takes 12 to 15 cycles for selecting an aptamer. Despite its iterative and time-consuming cycles, the selection often fails. One of the important factors is selection biases caused by PCR bias, non-specific sequence amplification, and the removal of high-affinity sequences. Resultantly, low-affinity or nonspecific candidates may have an overwhelmingly higher number in the final enriched aptamer library compared to high-affinity ones.

Existing methods of aptamer selection can be simply classified into two categories. The first category involves aptamer-target immobilization on the surface of a substrate. Most methods belong to this category, including filter membrane selection, plate surface selection, microbead surface selection, microarray surface selection, etc. Molecular binding is determined by both association and dissociation. As the selection is carried out on the surface, it is inevitable to lose high-affinity candidates once they are dissociated from the surface during the washing and enrichment step. Another major concern is the nonspecific binding of sequences to the substrate regardless of whether aptamer-target complexes are physically or chemically immobilized on the surface. As the nonspecific binding is random, the enrichment of nonspecific sequences is significant despite negative selection. The second category is based on the motion of aptamers, targets, and their complexes. Under electrical fields, their mobility shifts are different due to their different sizes, charges, and molecular weights. However, the use of specific electrolyte buffers and electrical fields can change the functional structures of proteins and aptamer candidates. The nonspecific binding of candidates to substrates is also problematic. Thus, these methods still require multiple cycles of selection and are rarely used for aptamer selection. It is also important to note that many methods require specific expertise or instruments (e.g., flow cytometer and capillary electrophoresis system) for separating candidate sequences.

Given the interest in aptamers, there remains a need for alternative materials and methods for aptamer selection. The compositions and methods disclosed herein address these and other needs.

### SUMMARY

Provided herein are aptamer-target complexes. These complexes can include an aptamer; and a target. The target can be conjugated to a porous hydrogel. The aptamer can selectively associate with the target. For example, in some embodiments, the aptamer can exhibit an affinity constant (Kₐ) of greater than about 10⁵ M⁻¹ with the target, such as greater than about 10⁶ M⁻¹, greater than about 10⁷ M⁻¹, greater than about 10⁸ M⁻¹, greater than about 10⁹ M⁻¹, greater than about 10¹⁰ M⁻¹, greater than about 10¹¹ M⁻¹, or greater than about 10¹² M⁻¹.

Also described herein are methods for identifying or producing an aptamer to a target. These methods can include: contacting a candidate mixture of nucleic acids with the target, wherein nucleic acids having affinity to the target bind the target and form nucleic acid-target complexes; partitioning nucleic acid-target complexes from free nucleic acids in the candidate mixture; amplifying the nucleic acids that bind to the target; and identifying nucleic acids that bind to the target, thereby identifying or producing an aptamer to a target; wherein the target is conjugated to a porous hydrogel.

In some embodiments, the aptamers identified using the methods described herein can be used to develop biomaterials for regenerative medicine or immunotherapy applications.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIG. 1 shows an illustration of hydrogel-based aptamer selection (HAS). Target molecules are immobilized in the macroporous hydrogel. Through the three-dimensional diffusion-binding process, low-affinity or medium-affinity candidates are quickly removed from the hydrogel while high-affinity candidates are well-retained in the hydrogel. The sizes and dimensions of pores, targets, and aptamer candidates in the cartoon do not reflect their real sizes and dimensions.

FIGS. 2A-2C show the process validation. Comparison between modeling analysis and experimental measurement. (2A) Simulation of aptamer diffusion in a 3 dimension (3-D) porous hydrogel. Simulation was performed with COMSOL software. The arrow indicates the increase of binding affinity (from 10 μM to 1 μM, 100 nM, 10 nM, and 1 nM). (2B) Fluorescence images of porous hydrogels conjugated with thrombin. FAM-labeled DNA 60-18 aptamer; Cy5 labeled-control aptamer. (2C) Time-dependent retention of thrombin aptamer in porous hydrogels conjugated with thrombin.

FIGS. 3A-3H shows hydrogel characterization and aptamer selection. (3A) Scheme of hydrogel synthesis through cryogelation. (3B) Confocal images of macroporous PEG hydrogel. Scale bar, 200 μm. (3C) SEM images of macroporous PEG hydrogel. Scale bar, 200 μm (top) and 50 μm (bottom). (3D) Pore size distribution of hydrogel examined via SEM. (3E) Examination of hydrogel swelling (n=6). (3F) Scheme of protein conjugation into hydrogel through carbodiimide reaction. (3G) Examination of conjugation efficiency. The initial amount of protein loaded into the hydrogel for conjugation is defined as 100% (n=3). (3H) FTIR spectra of PEG hydrogels with proteins (PEG-PRT).

FIGS. 4A-4B show the retention kinetics of aptamer library in native PEG hydrogels and PEG hydrogels functionalized with protein targets. (4A) shows polyacrylamide gel electrophoresis images of PCR amplified product of diffused aptamer library (ssDNA) from hydrogel-protein (IL10, IL12 and Thrombin) and hydrogel as control at different time intervals (h). (4B) Retention kinetics profile, release of ssDNA of aptamer library from protein-hydrogel (IL10, IL12 and Thrombin) and hydrogel (PEG) as control.

FIGS. 5A-5C show the characterization of HAS enriched library. SPR sensogram for enriched aptamer library with HAS and native aptamer library (Lib) with surface immobilized proteins, IL10 (5A), IL12 (5B) and Thr (5C).

FIG. 6 show the workflow for next generation sequencing (NGS) data analysis and docking for IL10, IL12 and thrombin aptamer selection. NGS data analysis pipeline, data analysis was performed with GALXY online tools (https://usegalaxy.org/). Totally, 178,233 NGS reads were acquired. After processing, 5,905 sequences (6,489 reads) were obtained. The top 50 sequences represented a total of 300 reads (4.63% of the 6,489 reads).

FIGS. 7A-7C show the SPR signals of aptamer candidates for IL10. (7A): one-round 60-our HAS selection. (7B): two-round 60-hour HAS selection. (7C): (2×T) one-round 120-hour HAS selection. IL10. X indicates the number of aptamer candidates. The SPR experiments were performed over three individual SPR chips.

FIGS. 8A-8B show the SPR signals of aptamer candidates for mIL12 (8A) and thrombin (T) (8B). The aptamer candidates were selected with one-round HAS selection.

FIGS. 9A-9D show binding analysis of T.7 aptamers with SPR. (9A) SPR sensorgram of T.7. The concentration was varied from 2.5 to 50 nM. (9B) Maximum SPR signal versus each concentration. (9C) Comparison of different aptamers including truncated T.7 aptamers, aptamer library, and two previously reported aptamers (29mer and 15mer) using SPR (n=2). The SPR signals were normalized by molecular weight of each aptamer. The numbers in the parenthesis show the length of each aptamer sequence. (9D) Representative SPR sensorgrams of truncated aptamers including four T.7 truncated aptamers, 60-18 (29) aptamer (29mer), and 15mer. Aptamer concentration, 10 nM. (9E) Secondary structure of four truncated aptamers including T7 (40), T7 (46), T (59) and T7 (71). In (9A-9C, and 9D), data are presented as mean values+/−SD.

FIGS. 10A-10B show binding analysis of mIL12 aptamer using SPR. (10A) SPR sensorgram. The concentration was varied from 2.5 nM to 50 nM. (10B) Maximum SPR signal versus each concentration.

FIGS. 11A-11D show binding analysis of IL-10 aptamers using SPR. (11A) IL10.49; (11B) 2T. IL10.13; (11C) 2.IL10.3. The concentration was varied from 2.5 nM to 50 nM. (11D) Binding affinity comparison of obtained IL10 aptamers from various cycle Maximum SPR signal versus concentration.

FIG. 12 shows the final selected aptamer sequences with respective secondary structure and measured K_{d} values of selected aptamers.

FIG. 13A-13C show biosensing demonstration of selected aptamer and specificity. (13A) Schematic illustration of antisense displacement assay with thrombin (T.7) aptamer. (13B) Calibration curve of T (thrombin) in 1×PBS buffer. (13C) Examination of aptamer specificity against various analogous proteins at 100 nM concentration (TNF-α: tumor necrosis factor; IFN-λ: Interferon gamma; Interleukin 7, Interleukin 6, HSA: human serum albumin).

FIG. 14A-14E shows selection and characterization of aptamers for GM-CSF, IL-12, IL-10, and BDNF. (14A) Gel electrophoresis images of the aptamer library released at different time points during the process of aptamer selection. (14B) Electrophoretic mobility shift assay (EMSA) for comparing the initial aptamer library and enriched aptamer pools in binding to target proteins. HAS: enriched aptamer pool acquired after one-step HAS; LIB: initial aptamer library. (14C) SPR analysis of enriched aptamer pools and the initial library in binding to target proteins. (14D) Binding analysis of representative selected aptamer candidates with SPR (n=2). The aptamer concentration was varied from 2.5 to 50 nM. Data are presented as mean values+/−SD. (14E) Secondary structures of the representative selected aptamers.

FIG. 15A-15B Numerical simulation of aptamer diffusion. (15A) Aptamer retention in the porous hydrogel at 60 h. (15B) Aptamer (Kd, 10 nM) distribution in the porous hydrogel at different time points.

FIG. 16A-16B Rheological characterization of the porous PEG hydrogel. (16A) Strain sweep. (16B) Frequency sweep. G′: Storage modulus, G″: Loss modulus.

FIG. 17A-17C (17A) Gel electrophoresis mobility shift analysis of the enriched aptamer pool and the initial library in binding to thrombin. LIB+T: thrombin mixed with the library; HAS+T: thrombin mixed with the enriched aptamer pool acquired after one-step HAS. This experiment has been done in triplicate with similar results. (17B) Release of the enriched pool (HAS) and the initial aptamer library (LIB) from the thrombin-conjugated porous PEG hydrogel. The same amounts of HAS and LIB were loaded into the PEG hydrogels. The sequences released from the PEG hydrogel were PCR-amplified under the same working condition and then analyzed with polyacrylamide gel electrophoresis. SYBR GOLD was used to stain the polyacrylamide gel. L-(0.1×): DNA library or enrichment pool initially loaded into the thrombin-conjugated porous PEG hydrogel was diluted 10 times for gel electrophoresis. 30 s-(0.5×): released buffer collected during the first 30 s post loading was diluted 2 times for gel electrophoresis. Higher fluorescence intensity indicates faster release. Quantification of fluorescence intensity of DNA bands using ImageJ was used to calculate the release and retention percentage of HAS and LIB from the thrombin-conjugated porous PEG hydrogel. This experiment has been repeated twice independently with similar results. (17C) Comparison between the initial library and the eluted solutions collected at 6, 24, and 60 hours using SPR.

FIG. 18 Comparison between the initial aptamer library and the enriched aptamer pools. The enriched aptamer pools were acquired by using three hydrogels conjugated with 0.01 μg (0.01×), 0.1 μg (0.1×) and 1 μg (1×) thrombin. The pools were eluted from the hydrogels at 60 hours. The samples were analyzed under the same conditions. The profiles show the time-dependent association and dissociation. The data suggest that when the protein density is low enough, the conjugated proteins will not be able to retain enough desired aptamer candidates.

FIG. 19 Cladogram of aptamer candidates analyzed via Clustal Omega. Each family is represented by a blue column. In the cladogram, the first number represents the sequence ID followed by the output of a Multiple Sequence Alignment (MSA) index. It corresponds to the evolutionary distance between sequences as defined by their “length”. The sequence highlighted in yellow were selected for further analysis based on the maximum MSA score and Gibbs energy.

FIG. 20A-20B Examination and comparison of truncated aptamers. (20A) Fluorescence analysis of thrombin-immobilized microparticles. The microparticle aliquots were treated with FAM-labeled aptamers of the same concentration (2.5 μM) and then analyzed using flow cytometry. Control aptamer: a FAM-labeled sequence not binding to thrombin. (20B) Competition of FAM-T7 (40), FAM-29mer and FAM-15mer with heparin in binding to thrombin-immobilized microparticles.

FIG. 21A-21D shows selection and characterization of aptamers for GM-CSF, and BDNF. (21A-21B) Quantification of library retention in hydrogels conjugated with or without target protein (n=2 for (21A) GM-CSF and (26D) BDNF. (21C-21D) SPR analysis of aptamer candidates in binding to target proteins (n=2 for (21C, D). The concentration of the aptamers was 10 nM. Aptamer candidates with binding signals of approximately 10 or lower than 10 RU are treated as low-affinity candidates (or internal controls). All data are presented as mean values+/−SD.

Like reference symbols in the various drawings indicate like elements.

### DETAILED DESCRIPTION

A number of embodiments of the disclosure have been described. Nevertheless, it will be understood that various modifications may be made without departing from the spirit and scope of the invention. Accordingly, other embodiments are within the scope of the following claims.

### Definitions

To facilitate understanding of the disclosure set forth herein, a number of terms are defined below. Unless defined otherwise, all technical and scientific terms used herein generally have the same meaning as commonly understood by one of ordinary skill in the art to which this disclosure belongs. Publications cited herein and the materials for which they are cited are specifically incorporated by reference.

As used in this specification and the following claims, the terms “comprise” (as well as forms, derivatives, or variations thereof, such as “comprising” and “comprises”) and “include” (as well as forms, derivatives, or variations thereof, such as “including” and “includes”) are inclusive (i.e., open-ended) and do not exclude additional elements or steps. For example, the terms “comprise” and/or “comprising,” when used in this specification, specify the presence of stated features, integers, steps, operations, elements, and/or components, but do not preclude the presence or addition of one or more other features, integers, steps, operations, elements, components, and/or groups thereof. Other than where noted, all numbers expressing quantities of ingredients, reaction conditions, geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Accordingly, these terms are intended to not only cover the recited element(s) or step(s), but may also include other elements or steps not expressly recited. Furthermore, as used herein, the use of the terms “a”, “an”, and “the” when used in conjunction with an element may mean “one,” but it is also consistent with the meaning of “one or more,” “at least one,” and “one or more than one.” Therefore, an element preceded by “a” or “an” does not, without more constraints, preclude the existence of additional identical elements.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. A range may be construed to include the start and the end of the range. For example, a range of 10% to 20% (i.e., range of 10%-20%) can includes 10% and also includes 20%, and includes percentages in between 10% and 20%, unless explicitly stated otherwise herein.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

It is understood that when combinations, subsets, groups, etc. of elements are disclosed (e.g., combinations of components in a composition, or combinations of steps in a method), that while specific reference of each of the various individual and collective combinations and permutations of these elements may not be explicitly disclosed, each is specifically contemplated and described herein.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

As used herein, “nucleic acid,” “oligonucleotide,” and “polynucleotide” are used interchangeably to refer to a polymer of nucleotides of any length, and such nucleotides may include deoxyribonucleotides, ribonucleotides, and/or analogs or chemically modified deoxyribonucleotides or ribonucleotides. The terms “polynucleotide,” “oligonucleotide,” and “nucleic acid” include double- or single-stranded molecules as well as triple-helical molecules.

The polynucleotide sequence may be modified, for example, to enhance efficacy and/or to reduce immune responsivity, by using, for example, base modifications or end-capping. In other embodiments, an unmodified polynucleotide sequence is used. For example, the polynucleotide can be an RNA sequence or a DNA sequence. In some embodiments, the mRNA can include an optimized codon. By codon optimizing, the formation of secondary structures can be reduced and translational efficiency improved. In certain embodiments, the codon optimization includes GC enrichment of the coding region. In certain embodiments, the codon optimization includes codon quality enrichment of the coding region. In certain aspects, the mRNA can include one or more regions or parts, which act or function as an untranslated region (UTRs) of a gene. UTRs are transcribed but not translated. In mRNA, the 5′ UTR starts at the transcription start site and continues to the start codon but does not include the start codon. The 3′ UTR starts immediately following the stop codon and continues until the transcriptional termination signal. The use of human-derived UTRs may facilitate the expression of the polypeptide in cells. In some embodiments, the polynucleotide comprises at least one chemically modified nucleotide. In some embodiments, the at least one chemically modified nucleotide comprises a chemically modified nucleobase, a chemically modified ribose, a chemically modified phosphodiester linkage, or a combination thereof. In some embodiments, the polynucleotide sequence as used comprise modified nucleosides such as 5-methylcystonsine or psudouridine.

As used herein “modified” refers to a changed state or structure of a molecule of the invention. Molecules may be modified in many ways including chemically, structurally, and functionally. In one embodiment, the polynucleotides of the present invention are “chemically modified” by the introduction of non-natural nucleosides and/or nucleotides, e.g., as it relates to the natural ribonucleotides A, U, G, and C. Modifications of the nucleosides and/or nucleotides as used in the present invention may be naturally occurring (i.e. comprise a nucleotide and/or nucleoside other than the natural ribonucleotides A, U, G, and C) or may be artificial. Non-canonical nucleotides such as the cap structures are not considered “modified” although they differ from the chemical structure of A, G, C, and U ribonucleotides. As used herein, a “structural” modification is one in which two or more linked nucleosides are inserted, deleted, duplicated, inverted or randomized in a polynucleotide without significant chemical modification to the nucleotides themselves. Because chemical bonds will necessarily be broken and reformed to effect a structural modification, structural modifications are of a chemical nature and hence are chemical modifications. However, structural modifications will result in a different sequence of nucleotides. When the polynucleotides of the present invention are chemically and/or structurally modified, the polynucleotides may be referred to as “modified nucleotides”.

In some embodiments, the nucleic acids disclosed herein can include at least one chemically modified nucleotide. In some embodiments, the at least one chemically modified nucleotide comprises a chemically modified nucleobase, a chemically modified ribose, a chemically modified phosphodiester linkage, or a combination thereof.

In one embodiment, the at least one chemically modified nucleotide is a chemically modified nucleobase.

In one embodiment, the chemically modified nucleobase is selected from 5-formylcytidine (5fC), 5-methylcytidine (5meC), 5-methoxycytidine (5moC), 5-hydroxycytidine (5hoC), 5-hydroxymethylcytidine (5hmC), 5-formyluridine (5fU), 5-methyluridine (5-meU), 5-methoxyuridine (5moU), 5-carboxymethylesteruridine (5camU), pseudouridine (Ψ), N¹-methylpseudouridine (me¹Ψ), N⁶-methyladenosine (me⁶A), or thienoguanosine (^{th}G).

In some embodiments, the chemically modified nucleobase is 5-methoxyuridine (5moU). In some embodiments, the chemically modified nucleobase is pseudouridine (Ψ). In some embodiments, the chemically modified nucleobase is N¹-methylpseudouridine (me¹Ψ).

The structures of these modified nucleobases are shown below:

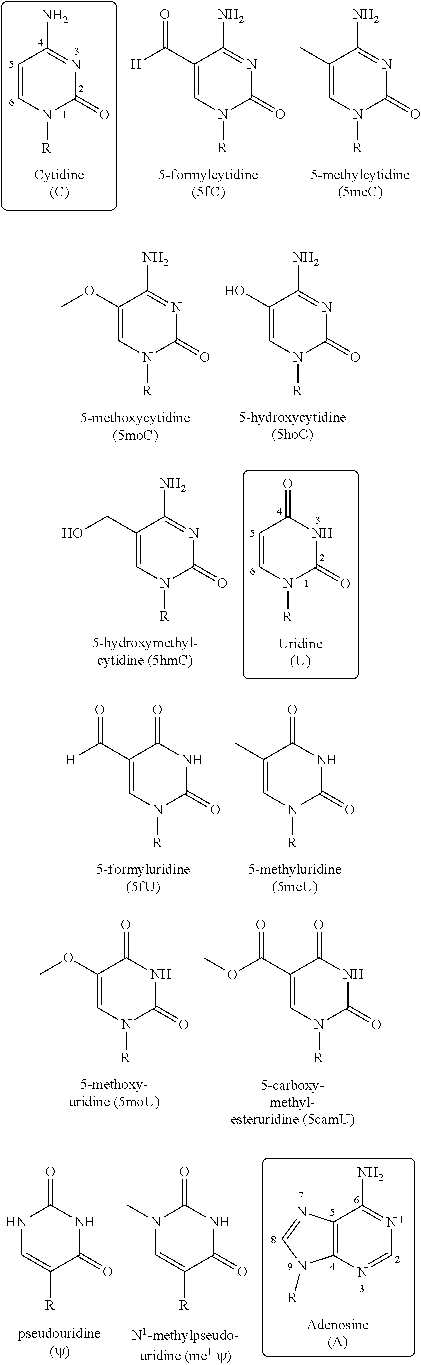

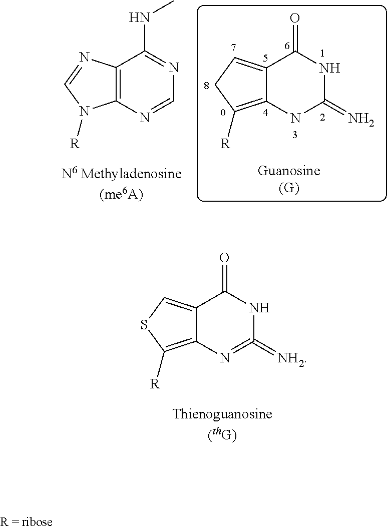

In one embodiment, the at least one chemically modified nucleotide is a chemically modified ribose.

In one embodiment, the chemically modified ribose is selected from 2′-O-methyl(2′-O-Me), 2′-Fluoro (2′-F), 2′-deoxy-2′-fluoro-beta-D-arabino-nucleic acid (2′F-ANA), 4′-S, 4′-SFANA, 2′-azido, UNA, 2′-O-methoxy-ethyl(2′-O-ME), 2′-O-Allyl, 2′-O-Ethylamine, 2′-O-Cyanoethyl, Locked nucleic acid (LAN), Methylene-cLAN, N-MeO-amino BNA, or N-MeO-aminooxy BNA. In one embodiment, the chemically modified ribose is 2′-O-methyl(2′-O-Me). In one embodiment, the chemically modified ribose is 2′-Fluoro (2′-F).

The structures of these modified riboses are shown below:

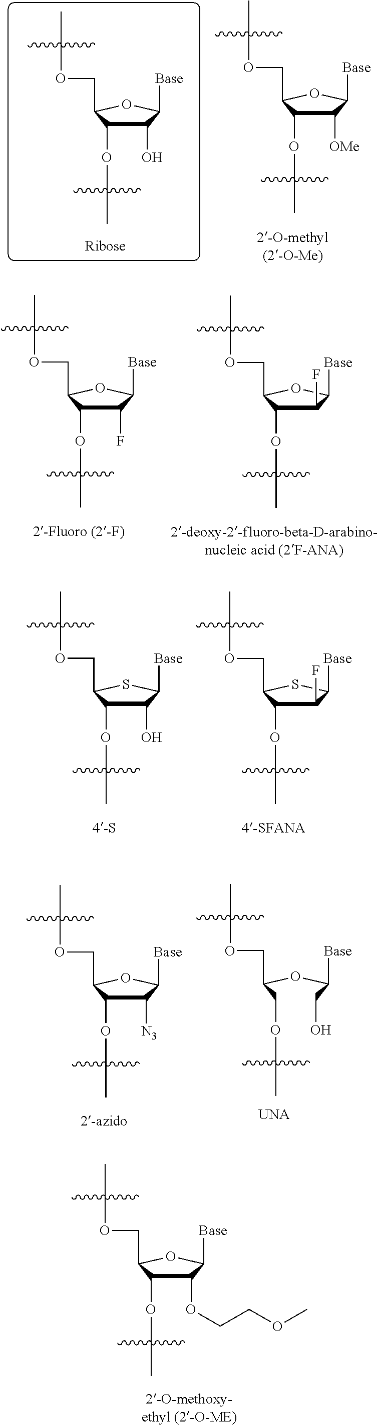

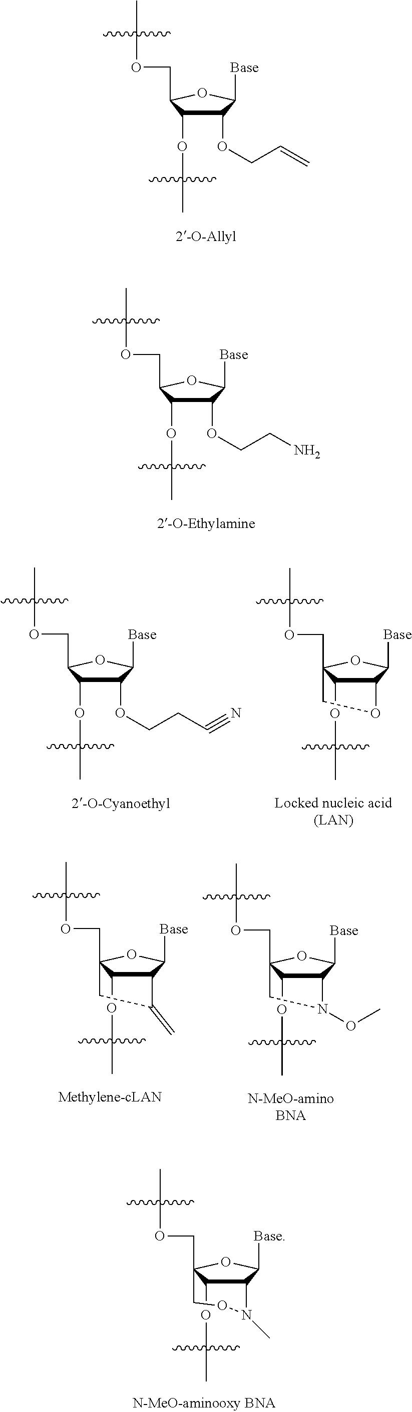

In one embodiment, the at least one chemically modified nucleotide is a chemically modified phosphodiester linkage.

In one embodiment, the chemically modified phosphodiester linkage is selected from phosphorothioate (PS), boranophosphate, phosphodithioate (PS2), 3′,5′-amide, N3′-phosphoramidate (NP), Phosphodiester (PO), or 2′,5′-phosphodiester (2′,5′-PO). In one embodiment, the chemically modified phosphodiester linkage is phosphorothioate.

The structures of these modified phosphodiester linkages are shown below:

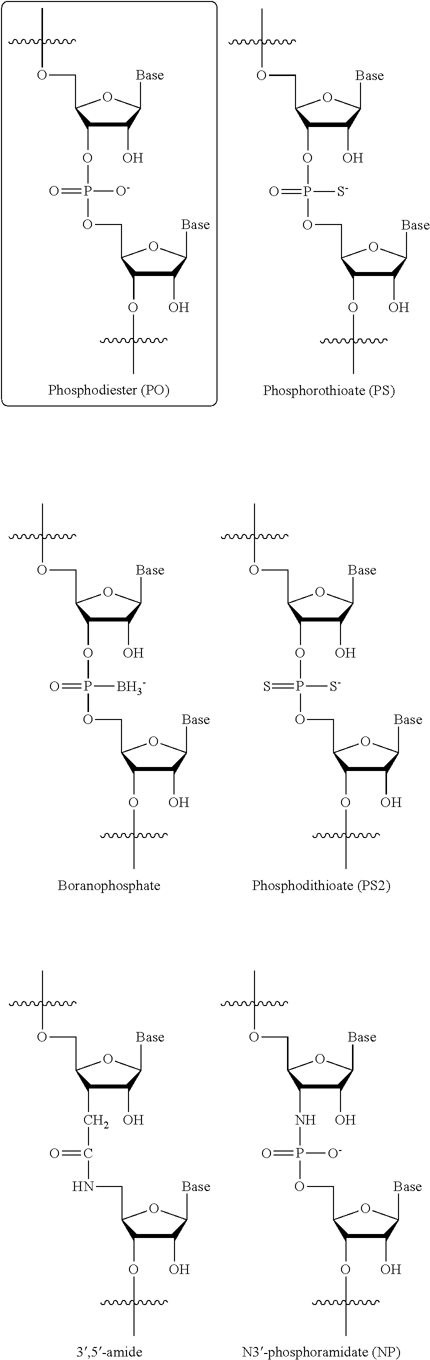

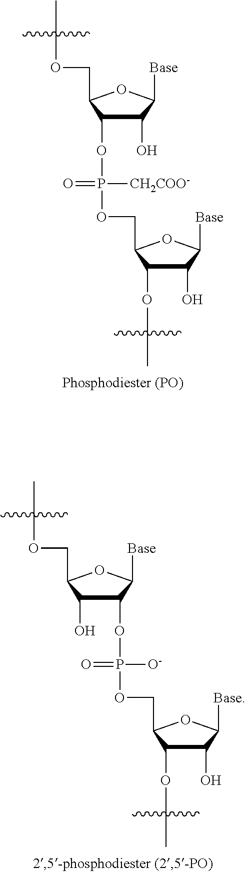

In some embodiments, the mRNA can include a heterologous 5′ untranslated region (5′UTR). In some embodiments, the mRNA can include a heterologous 3′ untranslated region (3′UTR).

If present, chemical modifications of a nucleotide can include, singly or in any combination, 2′-position sugar modifications, 5-position pyrimidine modifications (e.g., 5-(N-benzylcarboxyamide)-2′-deoxyuridine, 5-(N-isobutylcarboxyamide)-2′-deoxyuridine, 5-(N-tryptaminocarboxyamide)-2′-deoxyuridine, 5-(N-[1-(3-trimethylammonium) propyl]carboxyamide)-2′-deoxyuridine chloride, 5-(N-naphthylmethylcarboxamide)-2′-deoxyuridine, or 5-(N-[1-(2,3-dihydroxypropyl)]carboxyamide)-2′-deoxyuridine), modifications at exocyclic amines, substitution of 4-thiouridine, substitution of 5-bromo- or 5-iodo-uracil, backbone modifications, methylations, unusual base-pairing combinations such as the isobases, isocytidine and isoguanidine, and the like.

In one embodiment, the term “C-5 modified pyrimidine” refers to a pyrimidine with a modification at the C-5 position. Examples of a C-5 modified pyrimidine include those described in U.S. Pat. Nos. 5,719,273 and 5,945,527. Examples of a C-5 modification include substitution of deoxyuridine at the C-5 position with a substituent selected from: benzylcarboxyamide (alternatively benzylaminocarbonyl) (Bn), naphthylmethylcarboxyamide (alternatively naphthylmethylaminocarbonyl) (Nap), tryptaminocarboxyamide (alternatively tryptaminocarbonyl) (Trp), and isobutylcarboxyamide (alternatively isobutylaminocarbonyl) (iBu) as illustrated immediately below.

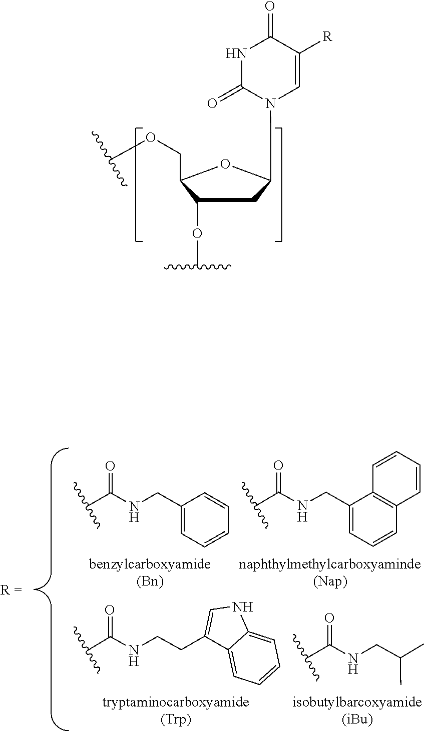

As delineated above, representative C-5 modified pyrimidines include: 5-(N-benzylcarboxyamide)-2′-deoxyuridine (BndU), 5-(N-isobutylcarboxyamide)-2′-deoxyuridine (iBudU), 5-(N-tryptaminocarboxyamide)-2′-deoxyuridine (TrpdU) and 5-(N-naphthylmethylcarboxyamide)-2′-deoxyuridine (NapdU).

Modifications can also include 3′ and 5′ modifications, such as capping or pegylation. Other modifications can include substitution of one or more of the naturally occurring nucleotides with an analog, internucleotide modifications such as, for example, those with uncharged linkages (e.g., methyl phosphonates, phosphotriesters, phosphoamidates, carbamates, etc.) and those with charged linkages (e.g., phosphorothioates, phosphorodithioates, etc.), those with intercalators (e.g., acridine, psoralen, etc.), those containing chelators (e.g., metals, radioactive metals, boron, oxidative metals, etc.), those containing alkylators, and those with modified linkages (e.g., alpha anomeric nucleic acids, etc.). Further, any of the hydroxyl groups ordinarily present in a sugar may be replaced by a phosphonate group or a phosphate group; protected by standard protecting groups; or activated to prepare additional linkages to additional nucleotides or to a solid support. The 5′ and 3′ terminal OH groups can be phosphorylated or substituted with amines, organic capping group moieties of from about 1 to about 20 carbon atoms, or organic capping group moieties of from about 1 to about 20 polyethylene glycol (PEG) polymers or other hydrophilic or hydrophobic biological or synthetic polymers. If present, a modification to the nucleotide structure may be imparted before or after assembly of a polymer. A sequence of nucleotides may be interrupted by non-nucleotide components. A polynucleotide may be further modified after polymerization, such as by conjugation with a labeling component.

Polynucleotides can also contain analogous forms of ribose or deoxyribose sugars that are generally known in the art, including 2′-O-methyl-, 2′-O-allyl, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside. As noted above, one or more phosphodiester linkages may be replaced by alternative linking groups. These alternative linking groups include embodiments wherein phosphate is replaced by P(O) S (“thioate”), P(S)S (“dithioate”), (O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalky, cycloalkenyl or araldyl. Not all linkages in a polynucleotide need be identical. Substitution of analogous forms of sugars, purines, and pyrimidines can be advantageous in designing a final product, as can alternative backbone structures like a polyamide backbone, for example.

In some embodiments, the aptamers can include at least additional one chemical modification selected from the group consisting of a 2′-position sugar modification, a 2′-amino (2′—NH2), a 2′-fluoro (2′-F), a 2′-O-methyl(2′-OMe) a modification at a cytosine exocyclic amine, a substitution of 5-bromouracil, a substitution of 5-bromodeoxyuridine, a substitution of 5-bromodeoxycytidine, a backbone modification, methylation, a 3′cap, and a 5′cap.

In some embodiments, a DNA encoded chemical library (DECL) chemically or physically linked with a small molecule, a protein, a peptide, a polysaccharide, or a lipid are used. This linkage allows the identity of the small molecule, protein, peptide, polysaccharide, or lipid to be determined by sequencing the associated DNA. It can be used in high-throughput screening for drug discovery, by attaching unique DNA tags to individual small molecules, proteins, peptides, polysaccharides, or lipids.

These modified nucleotides have been shown to produce novel aptamers that have very slow off-rates from their respective targets while maintaining high affinity to the target. In one embodiment, the C-5 position of the pyrimidine bases may be modified. Aptamers containing nucleotides with modified bases have a number of properties that are different than the properties of standard aptamers that include only naturally occurring nucleotides (i.e., unmodified nucleotides). In one embodiment, the method for modification of the nucleotides includes the use of an amide linkage. However, other suitable methods for modification may be used.

As used herein the term “amplification” or “amplifying” means any process or combination of process steps that increases the amount or number of copies of a molecule or class of molecules.

As used herein, “partitioning” means any process whereby one or more components of a mixture are separated from other components of the mixture. For example, aptamers bound to target molecules can be partitioned from other nucleic acids that are not bound to target molecules and from non-target molecules. More broadly stated, partitioning allows for the separation of all the nucleic acids in a candidate mixture into at least two pools based on their relative affinity and/or dissociation rate to the target molecule. Partitioning can be accomplished by various methods known in the art, including filtration, affinity chromatography, liquid-liquid partitioning, HPLC, etc. For example, nucleic acid-protein pairs can be bound to nitrocellulose filters while unbound nucleic acids are not. Columns that specifically retain nucleic acid-target complexes can also be used for partitioning. For example, oligonucleotides able to associate with a target molecule bound on a column allow the use of column chromatography for separating and isolating the highest affinity aptamers. Beads upon which target molecules are conjugated can also be used to partition aptamers in a mixture. If the beads are paramagnetic, the partitioning can be achieved through application of a magnetic field. Surface plasmon resonance technology can be used to partition nucleic acids in a mixture by immobilizing a target on a sensor chip and flowing the mixture over the chip, wherein those nucleic acids having affinity for the target can be bound to the target, and the remaining nucleic acids can be washed away. Liquid-liquid partitioning can be used as well as filtration gel retardation and density gradient centrifugation. Affinity tags on the target molecules can also be used to separate nucleic acid molecules bound to the tagged target from aptamers that are free in solution. For example, biotinylated target molecules, along with aptamers bound to them, can be sequestered from the solution of unbound nucleic acid sequences using streptavidin paramagnetic beads. Affinity tags can also be incorporated into the aptamer during preparation.

As used herein “binding” generally refers to the formation of a non-covalent association between the ligand and the target, although such binding is not necessarily reversible. The terms “nucleic acid-target complex” or “complex” or “affinity complex” are used to refer to the product of such non-covalent binding association.

“Polypeptide,” “peptide,” and “protein” are used interchangeably herein to refer to polymers of amino acids of any length. The polymer may be linear or branched, it may comprise modified amino acids, and/or it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. Polypeptides can be single chains or associated chains. “Marker” is used to describe a target molecule, frequently a protein, that is a specific indicator or predictor of a specific disease or condition for which a diagnosis is desired.

The term “polypeptide” refers to a compound made up of a single chain of D- or L-amino acids or a mixture of D- and L-amino acids joined by peptide bonds. A polypeptide is comprised of approximately twenty, standard naturally occurring amino acids, although natural and synthetic amino acids which are not members of the standard twenty amino acids may also be used. The standard twenty amino acids include alanine (Ala, A), arginine (Arg, R), asparagine (Asn, N), aspartic acid (Asp, D), cysteine (Cys, C), glutamine (Gln, Q), glutamic acid (Glu, E), glycine (Gly, G), histidine, (His, H), isoleucine (Ile, I), leucine (Leu, L), lysine (Lys, K), methionine (Met, M), phenylalanine (Phe, F), proline (Pro, P), serine (Ser, S), threonine (Thr, T), tryptophan (Trp, W), tyrosine (Tyr, Y), and valine (Val, V). The terms “polypeptide sequence” or “amino acid sequence” are an alphabetical representation of a polypeptide molecule.

Conservative substitutions of amino acids in proteins and polypeptides are known in the art. For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the polypeptides provided herein.

Substantial changes in protein function or immunological identity are made by selecting substitutions that are less conservative, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

The term “nucleobase” refers to the part of a nucleotide that bears the Watson/Crick base-pairing functionality. The most common naturally-occurring nucleobases, adenine (A), guanine (G), uracil (U), cytosine (C), and thymine (T) bear the hydrogen-bonding functionality that binds one nucleic acid strand to another in a sequence specific manner.

As used herein, “modified nucleic acid” refers to a nucleic acid sequence containing one or more modified nucleotides. In some embodiments it may be desirable that the modified nucleotides are compatible with the SELEX process.

“SELEX” refers to a process that combines the selection of nucleic acids that interact with a target in a desirable manner (e.g., binding to a protein) with the amplification of those selected nucleic acids. Optional iterative cycling of the selection/amplification steps allows selection of one or a small number of nucleic acids that interact most strongly with the target from a pool that contains a very large number of nucleic acids. Cycling of the selection/amplification procedure is continued until a selected goal is achieved. The SELEX methodology is described in the SELEX Patents. In some embodiments of the SELEX process, aptamers that bind non-covalently to their targets are generated. In other embodiments of the SELEX process, aptamers that bind covalently to their targets are generated. In some embodiments the targets used in the SELEX process are fixed in the same manner that an analytical sample would be fixed during the use of the slow off-rate aptamer in the histological or cytological characterization of that analytical sample.

“SELEX target” or “target molecule” or “target” refers herein to any compound upon which a nucleic acid can act in a desirable manner. A SELEX target molecule can be a protein, peptide, nucleic acid, carbohydrate, lipid, polysaccharide, glycoprotein, hormone, receptor, antigen, antibody, virus, pathogen, toxic substance, substrate, metabolite, transition state analog, cofactor, inhibitor, drug, dye, nutrient, growth factor, cell, tissue, any portion or fragment of any of the foregoing, etc., without limitation. Further the target may be modified in one or more fashion. For example, proteins may be modified by glycosylation, phosphorylation, acetylation, phospholipids, and so forth. The target may be modified to different levels. Slow off-rate aptamers could be produced to differentiate the type or level of modification. In one embodiment, a SELEX target does not include molecules that are known to bind nucleic acids, such as, for example, known nucleic acid binding proteins (e.g. transcription factors). Virtually any chemical or biological effector may be a suitable SELEX target. Molecules of any size can serve as SELEX targets. A target can also be modified in certain ways to enhance the likelihood or strength of an interaction between the target and the nucleic acid. A target can also include any minor variation of a particular compound or molecule, such as, in the case of a protein, for example, minor variations in amino acid sequence, disulfide bond formation, glycosylation, lipidation, acetylation, phosphorylation, or any other manipulation or modification, such as conjugation with a labeling component, which does not substantially alter the identity of the molecule. A “target molecule” or “target” is a set of copies of one type or species of molecule or multimolecular structure that is capable of binding to an aptamer. “Target molecules” or “targets” refer to more than one such set of molecules. Embodiments of the SELEX process in which the target is a peptide are described in U.S. Pat. No. 6,376,190, entitled “Modified SELEX Processes Without Purified Protein,” incorporated herein by reference in its entirety. FIG. 7 lists over 500 targets for which aptamers have been produced including a variety of slow off-rate aptamers. The target may also be a “marker” or a molecule that is indicative of a specific disease state or condition and may be used in the diagnosis of that specific disease state or for selection of an appropriate therapeutic regimen or as an indication of potential therapeutic efficacy. Examples of such markers include prostate specific antigen for prostate cancer, CMBK for heart disease, CEA, CA125 for cancer, HPV16 and HPV18 for cervical cancer, etc. An example of a marker that is predictive of therapeutic efficacy is HER2.

Reference will now be made in detail to specific aspects of the disclosed materials, compounds, compositions, articles, and methods, examples of which are illustrated in the accompanying Examples and Figures.

### Compositions

Described herein are aptamer-target complexes comprising an aptamer; and a target. In some embodiments, the aptamer can selectively associate with the target.

The term “selectively associates”, as used herein when referring to an aptamer, refers to a binding reaction which is determinative for the target in a heterogeneous population of other similar compounds. Generally, the interaction is dependent upon the presence of a particular structure (e.g., an antigenic determinant or epitope) on the binding partner. By way of example, an antibody or antibody fragment selectively associates to its particular target (e.g., an antibody specifically binds to an antigen) but it does not bind in a significant amount to other proteins present in the sample or to other proteins to which the antibody may come in contact in an organism. Likewise, an aptamer selectively associates to its particular target (e.g., an antibody specifically binds to an antigen) but it does not bind in a significant amount to other proteins present in the sample or to other proteins to which the antibody may come in contact in an organism.

In some embodiments, an aptamer that “specifically binds” a target has an affinity constant (Kₐ) greater than about 10⁵ M⁻¹ (e.g., greater than about 10⁶ M⁻¹, greater than about 10⁷ M⁻¹, greater than about 10⁸ M⁻¹, greater than about 10⁹ M⁻¹, greater than about 10¹⁰ M⁻¹, greater than about 10¹¹ M⁻¹, greater than about 10¹² M⁻¹, or more) with that target.

In some embodiments, the target can be conjugated to a porous hydrogel. In some embodiments, the target can be covalently bound to the porous hydrogel. The term “hydrogel” means a hydrophilic or amphiphilic three-dimensional polymer network capable of absorbing large amounts of water. Such networks can be composed of homopolymers or copolymers, and such networks are present in the presence of covalent chemical or physical (ionic, hydrophobic interactions, entanglement) crosslinks. Due to insolubility. Such cross-linking provides network structure and physical integrity. Hydrogels exhibit thermodynamic compatibility with water, so that the hydrogel can swell in aqueous media. The chains of the network are connected so that there are pores and a significant portion of such pores have dimensions of 1 nm to 100 nm.

In some embodiments, porous hydrogel can include a non-fouling polymer. “Non-fouling polymer” refers to any polymer that resists the absorption of proteins and/or adhesion of cells. In some embodiments, the non-fouling polymer can include a crosslinked hydrophilic polymer, zwitterionic polymer, mixtures or copolymers thereof. In some embodiments, the non-fouling polymer can include a crosslinked hydrophilic polymer. In some embodiments, the non-fouling polymer can include a zwitterionic polymer.

In some embodiments, porous hydrogel can include a zwitterionic polymer. Zwitterionic polymers refers to a family of materials that have the same number of cations and anions along their polymer chains. Typical cations are quaternized ammonium, and zwitterionic groups can be classified into sulfobetaine (SB), carboxybetaine (CB), phosphorylcholine (PC) according to anions. Suitable zwitterionic polymer can include, but are not limited to, examples of zwitterionic polymers: poly(methacryloyloxylethyl phosphorylcholine), polyMPC; poly(sulfobetaine methacrylate), polySBMA; and poly(sulfobetaineacrylamide), polySBAAm.

In some embodiments, porous hydrogel can include a crosslinked hydrophilic polymer. Suitable crosslinked hydrophilic polymers can include, but are not limited to, poly(ethylene glycol), polyoxazoline, polyaliphatic polyurethanes, polyether polyurethanes, polyester polyurethanes, polyethylene copolymers, polyamides, polyvinyl alcohols, poly(ethylene oxide), polypropylene oxide, polypropylene glycol, polytetramethylene oxide, polyvinyl pyrrolidone, polyacrylamide, poly(hydroxy ethyl acrylate), poly(hydroxy ethyl methacrylate), or mixtures or co-polymers thereof.

In some embodiments, the hydrophilic polymer can include poly(ethylene glycol) (PEG). In some embodiments, the PEG can be selected from: PEG-vinyl sulfone (PEG-VS), PEG-acrylate (PEG-Acr), PEG diacrylate, or mixtures or copolymers thereof.

In some embodiments, the porous hydrogel can have a pore size of from 10 nm to 200 μm, such as from 10 nm to 50 nm, from 10 nm to 1 μm, from 10 nm to 50 μm, from 10 nm to 100 μm, from 10 nm to 200 μm, from 50 nm to 1 μm, from 50 nm to 50 μm, from 50 nm to 100 μm, from 50 nm to 200 μm, from 1 μm to 20 μm, from 1 μm to 50 μm, from 1 μm to 100 μm, from 1 μm to 200 μm, from 50 μm to 100 μm, from 50 μm to 200 μm, or from 100 μm to 200 μm.

In some embodiments, the porous hydrogel can be a macroporous hydrogel. In some embodiments, the macroporous hydrogel can have a pore size of from 1 μm to 200 μm, such as from 1 μm to 20 μm, from 1 μm to 50 μm, from 1 μm to 100 μm, from 1 μm to 200 μm, from 10 μm to 50 μm, from 10 μm to 100 μm, from 10 μm to 200 μm, from 50 μm to 100 μm, from 50 μm to 200 μm, or from 100 μm to 200 μm.

In some embodiments, the macroporous hydrogel can have a porosity of from 30% to 90%, such as from 30% to 80%, from 30% to 70%, from 30% to 60%, from 30% to 50%, from 30% to 40%, from 40% to 80%, from 40% to 70%, from 40% to 60%, from 40% to 50%, from 50% to 80%, from 50% to 70%, from 50% to 60%, from 60% to 80%, from 60% to 70%, or from 70% to 80%.

Suitable methods for preparing the porous hydrogel are generally known in the art such as including but not limited to cryogelation; gas formation; salt leaching; or particle leaching. Different methods are known in the art for conjugating a target molecule such as proteins to a porous hydrogel network such as EDC/NHS, and click chemistry.

In some embodiments, the target can include a protein, a peptide, a receptor, an antibody, a growth factor, small molecule, toxins, polysaccharide, cell, bacteria, or virus. In some embodiments, the target can include a protein, a peptide, a receptor, an antibody, and a growth factor.

According to some aspects, the target can be an antibody such as cetuximab, anti-CD24 antibody, panitumumab, bevacizumab, immunoglobulins (e.g., IgG, IgA, IgM, IgD, IgE), and mixtures thereof. In some embodiments, the target can be growth factor such as transforming growth factor-α (“TGF-α”), transforming growth factors (“TGF-β”), platelet-derived growth factors (“PDGF”), fibroblast growth factors (“FGF”), including FGF acidic isoforms 1 and 2, FGF basic form 2 and FGF 4, 8, 9 and 10, nerve growth factors (“NGF”) including NGF 2.5 s, NGF 7.0s and beta NGF and neurotrophins, brain derived neurotrophic factor, cartilage derived factor, bone growth factors (BGF), basic fibroblast growth factor, insulin-like growth factor (IGF), vascular endothelial growth factor (VEGF), granulocyte colony stimulating factor (G-CSF), insulin like growth factor (IGF) I and II, hepatocyte growth factor, glial neurotrophic growth factor (GDNF), stem cell factor (SCF), keratinocyte growth factor (KGF), transforming growth factors (TGF), including TGFs alpha, beta, beta1, beta2, beta3, skeletal growth factor, bone matrix derived growth factors, and bone derived growth factors and mixtures thereof.

In some embodiments, the target can be a protein such as cytokines, cardiotrophin, stromal cell derived factor, macrophage derived chemokine (MDC), melanoma growth stimulatory activity (MGSA), macrophage inflammatory proteins 1 alpha (MIP-1 alpha), 2, 3 alpha, 3 beta, 4 and 5, IL-1, IL-2, IL-3, IL-4, IL-5, IL-6, IL-7, IL-8, IL-9, IL-10, IL-11, IL-12, IL-13, TNF-α, TNF-β, VEGF (vascular endothelial growth factor), NGFs (nerve growth factors), PDGF-AA, PDGF-BB, PDGF-AB, FGFb, FGFa, BGF, leptin, leukemia inhibitory factor (LIF), tumor necrosis factor alpha and beta, endostatin, thrombospondin, osteogenic protein-1, bone morphogenetic proteins 2 and 7, osteonectin, somatomedin-like peptide, osteocalcin, interferon alpha, interferon alpha A, interferon beta, interferon gamma, interferon 1 alpha, and interleukins 15, 16, 17 and 18. For example, in some embodiments, the target can be selected from: IL-10, IL-12, and thrombin.

The aptamer can be any non-naturally occurring nucleic acid that has or may have a desirable action on a target. A desirable action includes, but is not limited to, binding of the target, catalytically changing the target, reacting with the target in a way that modifies or alters the target or the functional activity of the target, covalently attaching to the target (as in a suicide inhibitor), and facilitating the reaction between the target and another molecule. In one embodiment, the action is specific binding affinity for a target molecule, such target molecule being a three-dimensional chemical structure, other than a polynucleotide, that binds to the aptamer through a mechanism which is predominantly independent of Watson/Crick base pairing or triple helix binding, wherein the aptamer is not a nucleic acid having the known physiological function of being bound by the target molecule. Aptamers include nucleic acids that are identified from a contacting a candidate mixture of nucleic acids with the target, wherein nucleic acids having affinity to the target bind the target and form nucleic acid-target complexes; partitioning nucleic acid-target complexes from free nucleic acids in the candidate mixture; amplifying the nucleic acids that bind to the target; and identifying nucleic acids that bind to the target, thereby identifying or producing an aptamer to a target; wherein the target is conjugated to a porous hydrogel.

It is recognized that affinity interactions are a matter of degree; however, in this context, the “specific binding affinity” or “selective association” of an aptamer for its target means that the aptamer binds to its target generally with a much higher degree of affinity than it binds to other, non-target, and components in a mixture or sample. An “aptamer” or “nucleic acid ligand” is a set of copies of one type or species of nucleic acid molecule that has a particular nucleotide sequence. An aptamer can include any suitable number of nucleotides. “Aptamers” refer to more than one such set of molecules. Different aptamers may have either the same number or a different number of nucleotides. Aptamers may be DNA or RNA and may be single stranded, double stranded, or contain double stranded regions.

In some embodiments, the aptamer can be a single-stranded nucleic acid or a double-stranded nucleic acid. In some embodiments, the aptamer can be a single-stranded nucleic acid. In some embodiments, the aptamer can be a double-stranded nucleic acid. In some embodiments, the aptamer can include DNA, RNA, or both DNA and RNA. In some embodiments, the aptamers can include DNA. In some embodiments, the aptamers can include RNA. In some embodiments, the aptamers can include both DNA and RNA.

In some embodiments, the aptamer can include a detectable moiety. Suitable detectable moieties can include but are not limited to a dye, a quantum dot, a radiolabel, an electrochemical functional group, an enzyme, an enzyme substrate, a ligand and a receptor.

As used herein, the term “labeling agent,” “label,” or “detectable moiety,” or “detectable element” or “detectable component” refers to one or more reagents that can be used to detect a target molecule/aptamer complex. A detectable moiety or label is capable of being detected directly or indirectly. In general, any reporter molecule that is detectable can be a label. Labels include, for example, (i) reporter molecules that can be detected directly by virtue of generating a signal, (ii) specific binding pair members that may be detected indirectly by subsequent binding to a cognate that contains a reporter molecule, (iii) mass tags detectable by mass spectrometry, (iv) oligonucleotide primers that can provide a template for amplification or ligation, and (v) a specific polynucleotide sequence or recognition sequence that can act as a ligand, such as, for example, a repressor protein, wherein in the latter two instances the oligonucleotide primer or repressor protein will have, or be capable of having, a reporter molecule, and so forth. The reporter molecule can be a catalyst, such as an enzyme, a polynucleotide coding for a catalyst, promoter, dye, fluorescent molecule, quantum dot, chemiluminescent molecule, coenzyme, enzyme substrate, radioactive group, a small organic molecule, amplifiable polynucleotide sequence, a particle such as latex or carbon particle, metal sol, crystallite, liposome, cell, etc., which may or may not be further labeled with a dye, catalyst or other detectable group, a mass tag that alters the weight of the molecule to which it is conjugated for mass spectrometry purposes, and the like. The label can be selected from electromagnetic or electrochemical materials. In one embodiment, the detectable label is a fluorescent dye. Other labels and labeling schemes will be evident to one skilled in the art based on the disclosure herein.

A detectable moiety (element or component) can include any of the reporter molecules listed above and any other chemical or component that may be used in any manner to generate a detectable signal. The detectable moiety, or signal generating label, may be detected via a fluorescent signal, a chemiluminescent signal, or any other detectable signal that is dependent upon the identity of the moiety. In the case where the detectable moiety is an enzyme (for example, alkaline phosphatase), the signal may be generated in the presence of the enzyme substrate and any additional factors necessary for enzyme activity. In the case where the detectable moiety is an enzyme substrate, the signal may be generated in the presence of the enzyme and any additional factors necessary for enzyme activity. Suitable reagent configurations for attaching the detectable moiety to a target molecule include covalent attachment of the detectable moiety to the target molecule, non-covalent association of the detectable moiety with another labeling agent component that is covalently attached to the target molecule, and covalent attachment of the detectable moiety to a labeling agent component that is non-covalently associated with the target molecule.

Detectable moieties may be incorporated into an aptamer during synthesis by using labeled dNTPs, dyes that have been generated as phosphoramidites, or other chemistries that can be employed during oligonucleotide synthesis, or may be incorporated by modification of the final aptamer product after synthesis. Each aptamer may include multiple detectable moieties to enhance signal generation. When multiple targets from the same sample, for example a histological tissue section, are to be detected then each target specific aptamer may be produced with a unique detectable moiety for simultaneous analysis of multiple targets.

### Methods

Described herein are methods for identifying or producing an aptamer to a target, the method can include: contacting a candidate mixture of nucleic acids with the target, wherein nucleic acids having affinity to the target bind the target and form nucleic acid-target complexes; partitioning nucleic acid-target complexes from free nucleic acids in the candidate mixture; amplifying the nucleic acids that bind to the target; and identifying nucleic acids that bind to the target, thereby identifying or producing an aptamer to a target; wherein the target is conjugated to a porous hydrogel. In some embodiments, the method can further include amplifying the nucleic acids that bind to the target. In some embodiments, the methods for identifying or producing an aptamer to a target described herein can include: contacting a candidate mixture of nucleic acids with the target, wherein nucleic acids having affinity to the target bind the target and form nucleic acid-target complexes; partitioning nucleic acid-target complexes from free nucleic acids in the candidate mixture; amplifying the nucleic acids that bind to the target; and identifying nucleic acids that bind to the target, thereby identifying or producing an aptamer to a target; wherein the target is conjugated to a porous hydrogel.

In some embodiments, described herein are methods for identifying an aptamer to a target, the method can include: contacting a candidate mixture of nucleic acids with the target, wherein nucleic acids having affinity to the target bind the target and form nucleic acid-target complexes; partitioning nucleic acid-target complexes from free nucleic acids in the candidate mixture; amplifying the nucleic acids that bind to the target; and identifying nucleic acids that bind to the target, thereby identifying or producing an aptamer to a target; wherein the target is conjugated to a porous hydrogel.

In some embodiments, described herein are method of producing an aptamer to a target, the method can include: contacting a candidate mixture of nucleic acids with the target, wherein nucleic acids having affinity to the target bind the target and form nucleic acid-target complexes; partitioning nucleic acid-target complexes from free nucleic acids in the candidate mixture; amplifying the nucleic acids that bind to the target; and identifying nucleic acids that bind to the target, thereby identifying or producing an aptamer to a target; wherein the target is conjugated to a porous hydrogel.

As used herein, a candidate mixture is also sometimes referred to as a “pool” or a “library.” For example, an “RNA pool” refers to a candidate mixture comprised of RNA. As used herein, “candidate mixture” is a mixture of nucleic acids of differing sequence from which to select a desired ligand. The source of a candidate mixture can be from naturally occurring nucleic acids or fragments thereof, chemically synthesized nucleic acids, enzymatically synthesized nucleic acids or nucleic acids made by a combination of the foregoing techniques. Modified nucleotides, can be incorporated into the candidate mixture. In addition, a SELEX process can be used to produce a candidate mixture, that is, a first SELEX process experiment can be used to produce a ligand-enriched mixture of nucleic acids that is used as the candidate mixture in a second SELEX process experiment. A candidate mixture can also comprise nucleic acids with one or more common structural motifs.

A candidate mixture of nucleic acids, or a library of nucleic acids, may be produced by an enzymatic method using a solid phase. In one embodiment, this method comprises the same basic steps described above. In this case the goal is the synthesis of an antisense library and these libraries are produced with a 5′ biotin modification. All remaining synthetic processes are as described above. Once the synthetic library is prepared, the nucleic acids may be used in a primer extension mix containing one or more modified nucleotides to produce the final candidate mixture in a classic primer extension method.

In one embodiment, a method for producing a synthetic library of nucleic acids comprises: 1) synthesizing the nucleic acids; 2) deprotecting the nucleic acids; 3) purifying the nucleic acids; and 4) analyzing the nucleic acids. In the synthesis step, a monomer mixture is prepared where the ratio of the various nucleotides in the mix is optimized to yield equal ratios of each nucleotide in the final product. One or more of the monomers in the mixture may comprise a modified nucleotide. Amidite protection groups are used in this procedure and in one embodiment, the monomer concentration is 0.1 M. During synthesis, the five prime protecting group is retained in the product nucleic acid. Synthesis is conducted on a solid support (controlled pore glass, CPG) and at least about 80 cycles are completed to synthesize the final product.

After the synthesis process, the nucleic acid product is deprotected. A 1.0 M aqueous lysine buffer, pH 9.0 is employed to cleave apurinic sites while the product is retained on the support (controlled pore glass, CPG). These cleaved truncated sequences are washed away with deionized (dI) water two times. 500 μL of dI water are added after the two washes in preparation for the deprotection step. This step involves the treatment with 1.0 mL of t-butylamine: methanol:water, 1:1:2, for 5 hours at 70° C., followed by freezing, filtration, and evaporation to dryness. The nucleic acid product is purified based on the hydrophobicity of the protecting group on a PRP-3 HPLC column (Hamilton). Appropriate column fractions are collected and pooled, desalted, and evaporated to dryness to remove the volatile elution buffers. The final product is washed with water by a centrifugation process and then re-suspended. Finally, the resuspended material is treated to deprotect the final product. Final product is characterized by base composition, primer extension, and sequencing gel.

Aptamers may be synthesized by the same chemistry that is used for the synthesis of a library. However, instead of a mixture of nucleotides, one nucleotide is introduced at each step in the synthesis to control the final sequence generated by routine methods. Modified nucleotides may be introduced into the synthesis process at the desired positions in the sequence. Other functionalities may be introduced as desired using known chemical modifications of nucleotides.

In various embodiments, each nucleic acid in a candidate mixture may have fixed sequences on either side of a randomized region, to facilitate the amplification process. The nucleic acids in the candidate mixture of nucleic acids can each further comprise fixed regions or “tail” sequences at their 5′ and 3′ termini to prevent the formation of high molecular weight parasites during the amplification process.

In one embodiment, the variable region of the aptamer includes nucleotides that include modified bases. Certain modified aptamers may be used in any of the described methods, devices, and kits.

In some embodiments, the aptamers identified using the methods described herein can be use to develop biomaterials for regenerative medicine, immunotherapy applications, among others.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the methods described herein without departing from the concept, spirit and scope of the disclosure. More specifically, it will be apparent that certain agents which are both chemically related may be substituted for the agents described herein while the same or similar results would be achieved. All such similar substitutes and modifications apparent to those skilled in the art are deemed to be within the spirit, scope and concept of the disclosure as defined by the appended claims.

By way of non-limiting illustration, examples of certain embodiments of the present disclosure are given below.

### EXAMPLES

### Example 1

Aptamers are “chemical antibodies”. Aptamers are synthetic oligonucleotides with the functions of rivaling natural antibodies. Despite this potential, the selection of aptamers is extremely challenging.

A method that is fundamentally different from any of current-state-of-the-art methods in concept was explored. This method is established according to molecular diffusion and automatic partition in a three-dimensional non-fouling macroporous hydrogel. The success of developing this new method leads to aptamer selection in one-single step. Its success will accelerate various aptamer-based basic studies and biomedical applications.

The hydrogel for aptamer selection (HAS) method makes aptamer selection extremely easy. Target molecules were conjugated into a three-dimensional macroporous hydrogel. After the aptamer library is loaded into this hydrogel, the hydrogel is transferred to a selection buffer. After 60 hours, the hydrogel is taken out and the remaining aptamers are eluted from the hydrogel.

### Automatic Selection and Sequential Partition in a Three-Dimensional Space (FIG. 1).

Almost all methods used for aptamer selection today are performed on a surface, which only depends on binding association and dissociation. However, once a high-affinity aptamer candidate dissociates and leaves from the surface to the environment, it is difficult to come back to the surface for re-binding. That is one of the fundamental reasons why multiple cycles of selection are needed. By contrast, our method is a binding-diffusion procedure. When a high-affinity candidate dissociates from an immobilized target molecule, it has the opportunity to bind the next target molecule continuously in a three-dimensional space. This binding-diffusion procedure is virtually an automatic, iterative, and sequential one-step selection and partition process that occurs in one single step. Resultantly, the best candidates will stay within the three-dimensional hydrogel whereas those weaker candidates quickly leave and diffuse out of the hydrogel.

Our approach for HAS is schematically illustrated in FIG. 1. Macro-porous PEGMA/PEGDA cryo-hydrogel was prepared through cryofreezing (−20° C.) polymerization. Target protein was covalently linked to the hydrogel functionalized with acrylic acid crosslinked inside the hydrogel. Later the aptamer library was incubated with target and non-binder, or low binding affinity candidates partitioned from hydrogel-target through the diffusion. Binders, candidate with high binding affinity PCR amplified and sequenced over NGS platform and probable aptamer candidates were selected for further characterization. The essence of our method has relied on the well-efficient partitioning of non-binders from binders' candidates of aptamer library from target linked hydrogel. When high-affinity binder candidates dissociate from immobilized target molecules, these candidates continuously have the opportunity to bind targets in a three-dimensional space. This binding-diffusion procedure is virtually the same as an automatic numerous step-by-step selection and partition process. Resultantly, the best binder candidates stayed within the three-dimensional hydrogel whereas those weaker nonbinder candidates quickly leave and diffuse out of the hydrogel. Taking advantage of the limited practical diversity of aptamer library for candidate selection after NGS. For a random region of N-40 (nucleotide-40 mer) aptamer library, the theoretical diversity should be in order of 4⁴⁰~10²⁴. It means 10²⁴ different types of aptamer candidates should be present in the pool of aptamer library. However, diversity of random pool in working aptamer library is much lesser (10¹⁴⁻¹⁵) than the theoretical value due to practical synthesis limitation (Pobanz and Lupták, 2016). Moreover, many sequences from pool of aptamer library can adapt into a similar nucleic acid secondary structure and conformation (e.g., CGCGAT: ATCGCG and ACTGAC: GTCAGT both form similar structures beside different sequences). Consequently, there is a high probability of presence of similar structures and sequences in the pool of aptamer library. Thus, enrichment of identical candidates with multiple hits after stringent partitioning and one round PCR-amplification, suggests possible aptamer candidates against the target. Negative selection is routinely used for aptamer selection because nucleic acid sequences nonspecifically bind to the surface of substrates or blocking agents (e.g., bovine serum albumin). The probability of fortuitously developed aptamer against matrix or blocking agent is significant factor for low success rate of standard SELEX procedure, these limitations were considered. The nonspecific absorption was minimized by immobilization of target over PEG hydrogel i.e., well known for its excellent antifouling properties. As a result, selection, and enrichment of nonspecific candidates for target is dramatically minimized.

Selection in a non-fouling space without negative selection or complicated instruments. Negative selection is routinely used for aptamer selection because 1) it is inevitable for nonspecific nucleic acid sequences to bind the surface of substrates; and 2) the surface cannot be blocked by using another molecule (e.g., bovine serum albumin. If blocking molecules are used, aptamers will be selected against the blocking molecules.). In our method, the entire substrate is a macroporous polyethylene glycol (PEG) hydrogel. PEG is a well-known non-fouling polymer. The nonspecific binding of aptamer candidates to PEG is minimal compared to the substrates used in current state-of-the-art methods. Thus, the enrichment of nonspecific candidates will be dramatically minimized. In addition, the proposed method does not involve any complicated instruments like flow cytometers for particle sorting. It does not involve electric fields or specific electrolyte solutions that may dramatically change the functional conformations of proteins and aptamer candidates.

High diversity and short time. Our method does not change the diversity of the aptamer library. In another word, the number of candidates in the library will not be reduced. By contrast, the diversity of the library or the number of candidates in other methods such as bead-based aptamer pools are dramatically decreased. This decrease in diversity diminishes the opportunity for selecting high-quality aptamers. This one-step aptamer selection will take only several days rather than months to accomplish. In contrast, methods like membrane filtration take at least several months or much longer to identify aptamer candidates. It is because the selection procedure always has unexpected or unidentified problems with the iteration.

### Development of Macroporous Hydrogels and Evaluation of Aptamer Release

### Synthesis of Macroporous PEG Hydrogels.

Macroporous PEG hydrogels are the substrates to be used for aptamer selection. Two methods of preparing macroporous PEG hydrogels were established, which forms a solid foundation to investigate and establish the proposed method. The first method is free radical polymerization coupled with carbon dioxide formation. Free radical polymerization allows PEG FIG. 1. Illustration of the concept. diacrylate to crosslink to form a polymer network. But free radical polymerization itself does not lead to the formation of large pores within the hydrogel matrix. To produce large pores for downstream protein conjugation and aptamer selection, the prepolymer solution is mixed with acetic acid. When the solution is added into a mold containing sodium bicarbonate, carbon dioxide is produced to generate bubbles in the hydrogels. Thus, free radical polymerization coupled with carbon dioxide formation can lead to the development of macroporous PEG hydrogels. The effects of the concentration of PEG on the pore formation was examined. With the increase of the concentration from 5 to 20% (w/v), large and deep pores formed in the PEG hydrogels. The 15% and 20% PEG hydrogels exhibited similar morphology. The pores had a normal distribution ranging from 20 to 100 μm and with an average size of 55 μm. With these large pores, the macroporous hydrogels would allow for the quick release of low-affinity or nonspecific DNA/RNA sequences to ensure fast and effective aptamer selection. Another method for preparing macroporous hydrogels was also developed. It is a polymerization-freezing procedure. In this method, acetic acid and sodium bicarbonate are not involved. The formation of pores is based on the formation of ice crystals at −20° C. The melting of ice crystals leads to the formation of large pores.

Thus, two methods to for generating macroporous non-fouling PEG hydrogels for aptamer selection were established.

Diffusion and Release of High-Affinity and Low-Affinity Aptamers from Protein-Conjugated Macroporous Hydrogels.

The fundamental concept of this new method is that the apparent diffusion and release kinetics of candidates are different because of their different binding affinities.

Previous studies have shown that high-affinity ligands can dramatically reduce the apparent diffusion coefficients of proteins by several orders of magnitude^{19,27,28}. Before performing aptamer selection with the new method, a simple modeling analysis and an experiment to understand aptamer diffusion were performed. Herein the similar analysis of aptamer transport in a macroporous hydrogel based on a two-dimensional diffusion-binding model was demonstrated.

Nonspecific aptamer sequences in a porous hydrogel have apparent diffusion coefficients at the level of ~10⁻⁷ cm²/s. This can be further validated using aptamer release from a macroporous hydrogel. The aptamer release from PEG gel without thrombin and also from thrombin-conjugated PGE gel will be studied.

PEG hydrogels with acrylic acid monomers were synthesized. After activation of the carboxyl group with the standard carbodiimide reaction, it was possible to conjugate proteins to the PEG hydrogel network. FAM-labeled BSA was used as a model to illustrate the success of protein conjugation.

Thrombin was conjugated to the macroporous PEG hydrogel. After washing the free molecules out of the hydrogel, fluorophore-labeled anti-thrombin aptamers were loaded into the hydrogels and then incubated the hydrogel in a phosphate buffered solution (PBS). The release or retention of the aptamers was evaluated by examining the fluorescence intensity of the PBS supernatant and the PEG hydrogel.

The fluorescence intensity of PBS increased with time. Correspondingly, the fluorescence intensity of the hydrogels decreased with time. In comparison, the hydrogel with the high-affinity aptamer had much higher fluorescence intensity than those with the low-affinity or scrambled aptamer. These data demonstrate that the aptamers could be released from thrombin-conjugated macroporous PEG hydrogels. The data further suggest that the high-affinity aptamer could be retained in the thrombin-conjugated macroporous PEG hydrogel with a much higher level. By contrast, the retention of the low-affinity or scrambled aptamer was much lower. In particular, the scrambled aptamer exhibited a very significant burst release within the first day.

Taken together, the retention and release data strongly suggest that nonspecific binding or low-affinity aptamers in a DNA/RNA library will be released from a protein-conjugated macroporous PEG hydrogel very quickly. High-affinity aptamers will be retained in the macroporous PEG hydrogel.

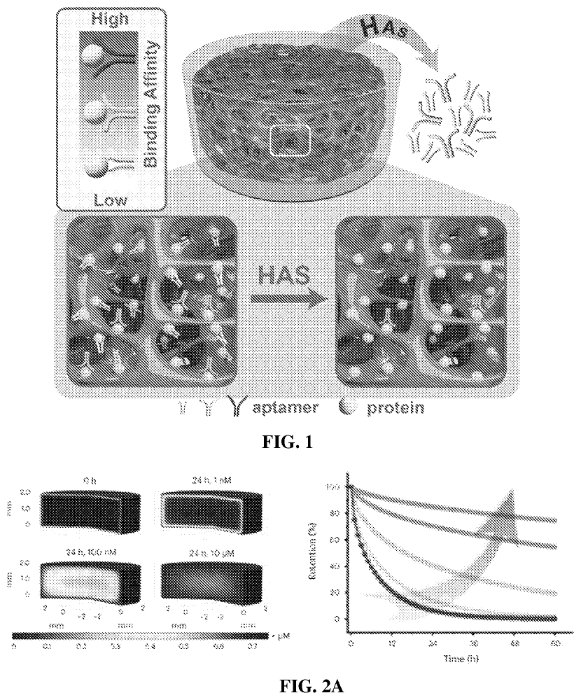

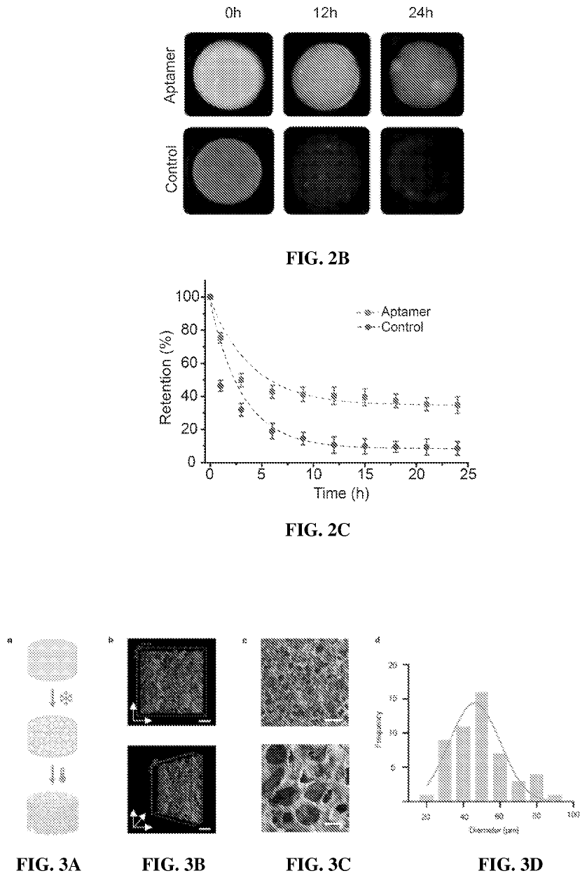

To support the applicability of the concept, the differential retention capacity of a target (protein) bound hydrogel for high, moderate, and low binding affinity aptamers was evaluated. Simulated the release of aptamer candidates from hydrogel with COMSOL. The concentration profile was shown in and at the surface of the hydrogel. This model predicts the diffusion of a biomolecule from the center to the surface (outer media) of hydrogel. Simulated result from COMSOL 3-D model was displayed as a diffusion profile over a period of 72 h (FIG. 2A). It is evident from the figure that aptamer candidates diffuse more rapidly in the absence of any affinity from hydrogel and complied with the established inverse relationship between binding affinity and diffusivity. Simulated diffusivity result for the release of biomolecules with different binding affinity was also shown in respective retention kinetics profiles.

The practical versatility of the concept was validated, thus fluorophore-labeled thrombin aptamer {T (DNA 60-18) 29 mer with Kd-0.5 nM (Tasset et al., 1997)} and its scrambled sequences were used to show the binding affinity-dependent retention profile. The thrombin-hydrogels exhibited strong fluorescence, confirming the successful aptamer interaction and incorporation into the hydrogel network. Protein-target specific binding and diffusion gradient in and out of the hydrogel network, thrombin aptamer (or scrambled) diffused outside of hydrogel into the buffer at a different rate. Which induced the decrease of aptamer concentration inside the hydrogel as depicted by a decrease in fluorescence intensity (FIG. 2B). The diffused aptamer concentration change in buffer was recorded at multiple time points until the equilibrium was obtained, the cumulative release of aptamers with time were shown in FIG. 2C. The cumulative release of high binding affinity aptamer (T29) was about 60% and in the case of respective scrambled aptamer cumulative release was increased to about 90%. The high cumulative release of scrambled aptamer was attributed to the diffusion effect and low binding affinity with a target.

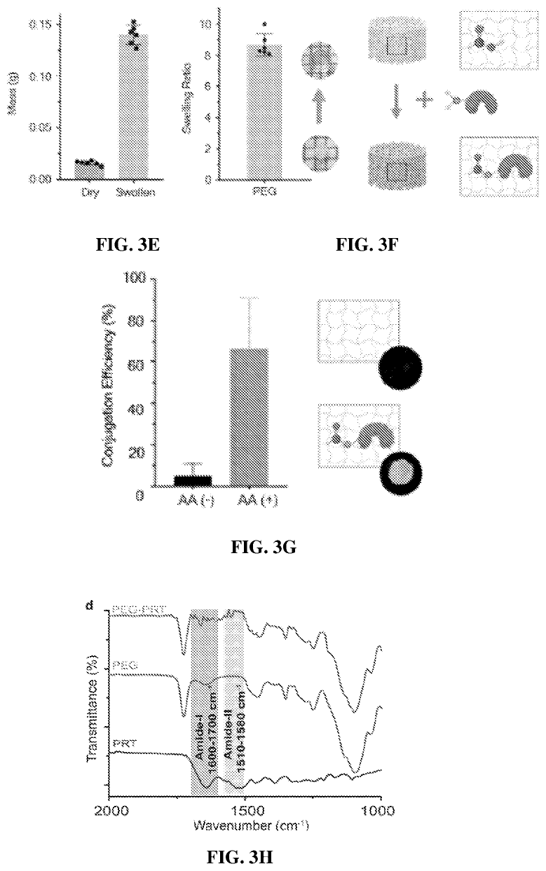

Macro porous PEGMA/PEGDA hydrogel was synthesized through free radical polymerization with a cryo-freezing procedure (FIG. 3A). Homogenous macro porous hydrogel (FIG. 3B, 3C, 3D) and porosity and pore structure was assessed via the confocal and bright field microscopy was achieved. The interconnected pores with clear boundaries were distributed throughout the hydrogel. The averaged pore diameter was 50±20 μm and calculated porosity ranged from 40% to 55%. The swealing ratio (FIG. 3E) of prepared hydrogel was demonstrated and schematic for conjugation (FIG. 3F) of target protein with hydrogel was depicted.

The incorporation of acrylic acid (AA) facilitates the covalent immobilization of the target protein on the hydrogel by providing the free carboxylic functional group. AA optimization study (FIG. 3G) was performed the with FITC-BSA immobilization through EDC/NHS chemistry. 1% AA in hydrogel was able to provide complete coverage of FITC-BSA over the hydrogel. Later the covalent interaction was confirmed through the FTIR (FIG. 3H). The covalent interaction between hydrogel and protein was confirmed by characteristic amide bond peaks at ~1670 cm-1 (from C═O group of amides), ~1540 cm-1 (—NH bending vibration of amine), it support the covalent interaction of protein with hydrogel. Besides the covalent binding between hydrogel and protein some other interactions such as dipole bond, hydrogen bond and electrostatic/ionic linkage cannot be ruled out. As a result of these interactions, the protein was prevented from percolating from the hydrogel under stringent diffusing conditions.

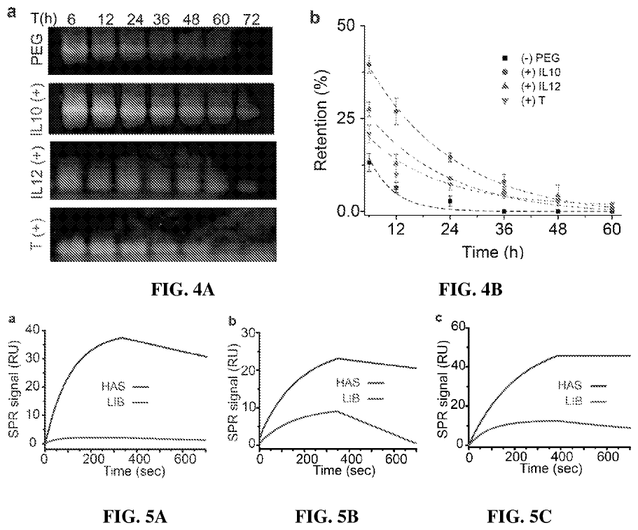

Protein (IL10, IL12, and Thrombin) conjugated hydrogel was subjected to the release of aptamer library. According to FIG. 4A, 4B, over 95% of the aptamer library's ssDNA was released from the protein-hydrogel after 60 hours, as opposed to the control hydrogel where all the molecules were released after 36 hours. The released samples were collected at different intervals and subjected them to PCR amplification and found similar results. These results clearly show that the release of ssDNA of aptamer library from protein hydrogel was significantly less compared to the control, which demonstrates that the protein hydrogel efficiently holds ssDNA with high binding affinity.

HAS was performed against the IL10, IL12, and thrombin and enriched the aptamer library with high-affinity aptamer candidates. The binding affinity of the enriched aptamer library was evaluated compared to native aptamer library through surface plasmon resonance (SPR) spectroscopy and electrophoretic mobility assay. The bulk dissociation constant (KD) was measured with SPR as shown in FIGS. 5A, 5C, and 5E, as expected enriched aptamer library displayed a very high binding affinity compared to the native library.

### Method for Aptamer Selection

### Experimental Design & Methods

A binding reaction can reduce the apparent diffusion of molecules. Thus, when aptamer candidates are loaded into protein-conjugated PEG hydrogels, those candidates with higher binding affinities will be released more slowly than those with lower binding affinities. Resultantly, higher-affinity aptamer candidates can be automatically separated from those low-affinity sequences through this binding-diffusion process.

Successful aptamer selection has the following requirements. The nonspecific binding of candidates to substrates for aptamer selection needs to be minimal. Proteins need to be easily conjugated with the hydrogel under mild conditions. The aptamer library needs to be easily loaded into the hydrogel. Those with low-binding affinities need to diffuse out of the hydrogel quickly. A macroporous PEG structure can satisfy all these requirements. Two methods of synthesizing macroporous PEG hydrogels to mitigate potential technical problems for aptamer selection to a great degree were established. method 1 will be use (i.e., polymerization coupled with gas formation) for aptamer selection and Method 2 (i.e., polymerization coupled with freezing/drying) as an alternative solution. Aptamers were selected against five proteins including human thrombin, human IL-10, murine IL-12, Brain derived neurotropic factor (BDNF) and Granulocyte Macrophage-Colony Stimulating Factor (GMCSF) (see Tables 1 and 3). The reasons for using these three proteins as models are as follows. 1) They have different isoelectric points (pI) that represents negatively, positively, and neutrally charged molecules, respectively. 2) The three model proteins are from different species (human and mouse). 3) The existing thrombin aptamer is probably the most studied aptamer in the literature. When new aptamers against thrombin are select, the new method with traditional ones can be compared. 4) These proteins are important for basic studies or biomedical applications such as biosensing or immunosuppression.

### Synthesis of Macroporous Hydrogels

Method 1: polymerization coupled with gas formation. The prepolymerization solution will contain PEGDA (15% w/v), acrylic acid (1% w/v), tetramethylethylenediamine (TEMED; 0.2% w/v), ammonium persulfate (APS; 0.4% w/v), and Pluronic F-127 (4% w/v). The prepolymerization solution will be transferred into a mold containing sodium bicarbonate to induce formation of macroporous hydrogels. Fully cured hydrogels will be washed using deionized water to remove unreacted molecules and initiator/catalysts. Macroporous hydrogels will be lyophilized and sterilized using 70% ethanol.

Method 2: polymerization coupled with freezing/drying. The prepolymerization solution will be incubated in ice before the addition of APS (0.4% w/v) and TEMED (0.2% v/v). The mixture will be poured into a 35 mm petri dish and the solution will be frozen at −20° C. overnight. The hydrogel will be removed from the freezer and allowed to fully thaw at room temperature. Hydrogels will be washed in distilled water to remove all unreacted monomers and initiator/catalysts. The macroporous hydrogels will be lyophilized and sterilized.

### Protein Conjugation

Covalent immobilization of protein can be achieved using the standard amine coupling method. In brief, the macroporous PEG hydrogel were incubated in a mixture of freshly prepared 30 mM 1-ethyl-3-(3-dimethylaminopropyl) carbodiimide hydrochloride (EDC) and 20 mM N-hydroxysuccinimide (NHS) in MES buffer for 1 h at 4° C. After gentle washing, the activated hydrogel was incubated with 1 μg of protein (300 μL) at 4° C. for 2 h. The hydrogel was gently washed with washing buffer (1×PBS, 0.2% Triton X, 300 mM NaCl) for 5 min (3 times) to remove any loosely/physically bound protein. The conjugated hydrogel can be stored at 4° C. until used. Unconjugated proteins in the washing solution were analyzed using ELISA to determine the total amount of conjugated proteins in the hydrogel network. Reaction parameters such as protein concentration can be varied.

### Aptamer Selection

A DNA aptamer library (5′-CACCTAATACGACTCACTATAGCGGATCCGA-N40-CTGGCTCGAACAAGCTTGC-3′ (SEQ ID NO: 1)) can be ordered from IDT and dissolved in binding buffer (1×PBS, 5 mM MgCl₂, 2.5 mM CaCl₂) and 2.5 mM KCl). The library was denatured at 95° C. for 5 min followed by cooling at room temperature for 10 min. The aptamer library in 400 μL binding buffer was incubated with the protein-conjugated PEG hydrogel with one-hour shaking. The hydrogel was then transferred to and incubated in 400 μL binding buffer. The supernatants was collected and replaced with fresh binding buffer solutions at an interval of 12 h for N times, where N can be 5 to 8. At the end of diffusion, all of the strongly bound aptamer candidates were eluted by incubation in 1M NaCl for 6 h. The eluted candidates were desalted with Zeba desalting column and concentrated until approximately ~50 μL solution was be obtained. The concentrated sequences were amplified and purified. The list of primer and aptamer library was incorporated in table 2.

TABLE 2
Aptamer library, primers, and antisense sequences for aptamer selection.

| Name | SEQ ID NO. | Sequence (5′-3′) |
|---|---|---|
| Aptamer | 52 | CACCTAATACGACTCACTATAGCGGATCCGA-N40- |
| Library | | CTGGCTCGAACAAGCTTGC |
| (N40) | | |
| Forward | 53 | CACCTAATACGACTCACTATAGCGGA |
| primer | | |
| Reverse | 54 | GCAAGCTTGTTCGAGCCAG |
| primer | | |
| Reverse | 55 | biotin-GCAAGCTTGTTCGAGCCAG |
| biotin | | |
| primer | | |
| Extended | 56 | AAAAAAAAAAAAAAAAAAAAAAAAAAAAAACACCTAA |
| forward | | TACGACTCACTATAGCGGA |
| primer | | |
| Extended | 57 | AAAAAAAAAAAAAAAAAAAAAAAAAAAAAAGCAAGC |
| reverse | | TTGTTCGAGCCAG |
| primer | | |
| Thrombin | 58 | GAT ATG GGA TCA TAG GTG |
| AS-18 | | |
| Thrombin | 59 | GGA TCA TAG GTG CAC CTG GCT |
| AS-21 | | |
| Thrombin | 60 | GGA TCA TAG GTG CAC CTG GCT CGA |
| AS-24 | | |
| T29 | 67 | CTC TTG GCC AAT TGG CCA ATC ATG CAT TA |
| Scrambled | | |
| Sscambled | 83 | GGAGAGAGAGAGAATATAAGAAAACAGCAGATCAGTCT |
| KK | | ATCTTCTCCTGATGGGTTCCTATTTATAGGTGAAGCTGT |

### Binding Analysis of Enriched Aptamer Pools

The binding affinities (represented by the K_{D} values) of the enriched aptamer pool were examined by using SPR spectroscopy according to the standard protocol developed in our lab. In brief, the sensor chip with carboxyl groups on the surface was activated using the standard amine coupling method. 100 μL of NHS and EDC mixture was injected over the biochip surface at a flow rate of 5 μL/min. Protein solution of 20 μg/mL was prepared in acetate buffer at a pH value close to the protein's isoelectric point. The protein solution was flowed over the activated surface at 10 μL/min for 5 min. 35 μL of ethanolamine was injected over the surface to deactivate the remaining reactive sites on the biochip. Hank's balanced salt solution (HBSS) with 20 mM HEPES buffer (pH 7.4) was used to rinse the chip. The aptamers were dissolved in the binding solution (HBSS with 4 mM CaCl₂), 4 mM MgCl₂, and 0.005% Tween 20) for the association analysis. The solution ran at a constant rate of 30 μL/min. The binding solution without the aptamers was used as washing buffer to analyze the dissociation. The kinetic parameters, kₒₙ and k_{off}, were determined by the direct curve fitting of the sensorgrams using the Reichert's Autolink software. The dissociation equilibrium constant, K_{D}, was calculated as k_{off}/kₒₙ.

### Results

Low-affinity DNA sequences quickly diffuse out of protein-conjugated macroporous PEG hydrogels. The enriched aptamer pool binds to target proteins with a much lower K_{D} value than the initial aptamer library. As techniques of preparing hydrogels and conjugating proteins are developed. Selection of aptamers after 60-hour diffusion is intended. This period of time for diffusion can be prolong to two-rounds of 60-hour selection, or increase the stringency of selection (e.g., using chaotropic agents). The proposed method can be combined with traditional methods. Results for these experiments can be found in FIGS. 3A-3D, 4A-4B, 5A and 5C-5D.

### Determine the Sequences and Functions of Selected Aptamers

### Experimental Design & Methods

The purpose of aptamer selection is to reduce the number of candidates (i.e., the enrichment of high-affinity aptamer candidates). After the selection, one needs to find the exact sequences of aptamer candidates and further validate the binding functions of the identified aptamers. Thus, aptamer candidates will undergo downstream sequencing and functional validation.

Experiments were performed for next generation sequencing and bioinformatics analysis. After sequencing, aptamer candidates were be truncated, and their capability of binding their target molecules was examined using SPR. Besides the SPR analysis, individual aptamers can be further analyzed using functional coagulation or cell assays.

### Aptamer Sequencing

Purified sequences were analyzed using Illumina's next generation sequencing platform with distinct prespecified barcodes and sequenced from the Azenta USA. The raw data obtained in the FASTQ format were imported into Galaxy web server for analysis. An overall evaluation of the data and general statistics was be obtained using the “FASTQC and Trimmometric” tool. The format of the data was then converted to FASTA. The length of the sequences was constrained to 80-100 bp to remove amplification and sequencing artifacts from the data sets. Unique sequences were then identified using the “Collapse” tool. Abundant sequences were identified, analyzed, and characterized. The most abundant sequences from last step (72 h) of the eluted product were identified after trimming, QC, collapsing, analyzed and characterized as mentioned in detail under the supporting information. Finally, the top 50 selected candidates based on abundance hit were identified as potential binders with target protein.

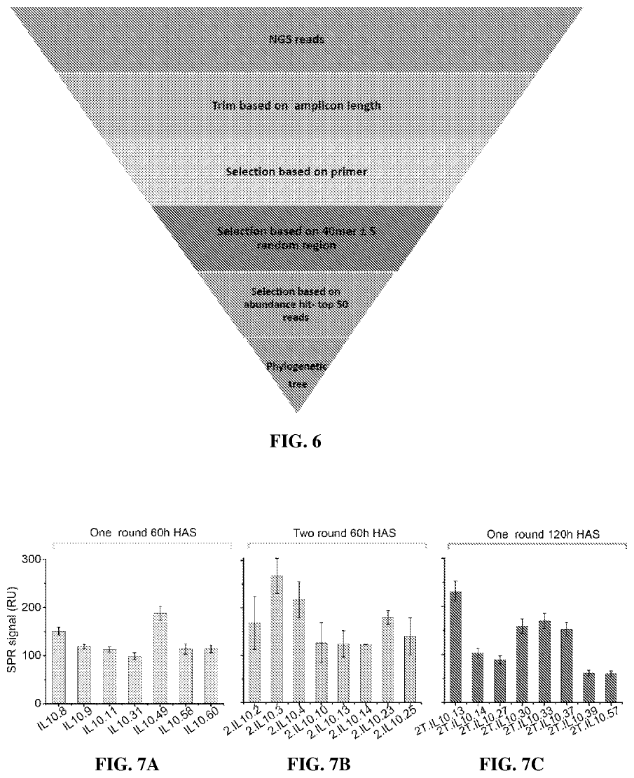

Multiple sequence alignment of these sequences using the external tool Clustal Omega (https://www.ebi.ac.uk/Tools/msa/clustalo/) was finally performed to identify sequence families. Based on this information, single candidates from each family were chosen based of Gibbs energy for further bioinformatics analysis. See FIG. 6.

### Aptamer Truncation

Truncation of non-essential nucleotides will be performed using a secondary structure generated by mFold/RNA structure online tool. The secondary structures and Gibbs energy of truncated aptamers will be analyzed and compared with parent aptamers using computer algorithms.

### Binding Analysis of Selected and Truncated Aptamers

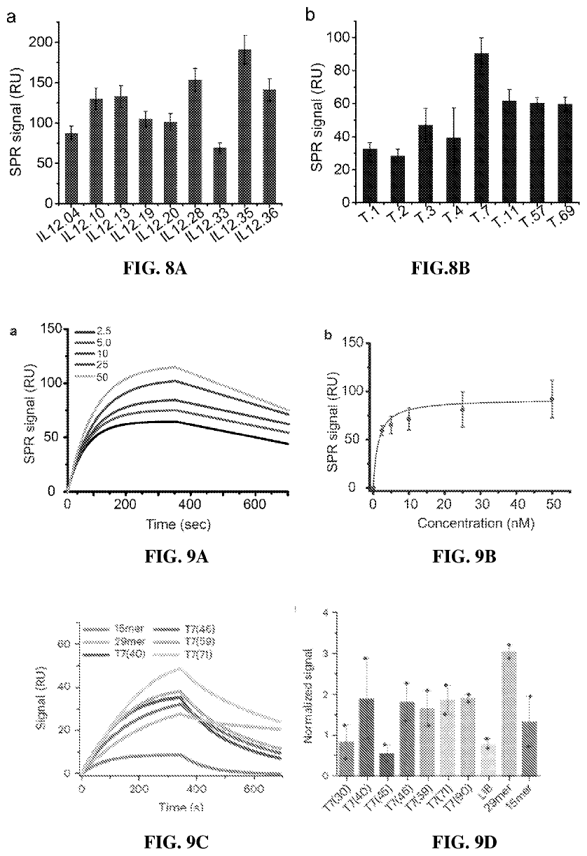

Binding affinities of selected and truncated aptamers will be measured using surface plasmon resonance (SPR) spectroscopy as previously described. In addition, biological assays will be performed to examine the binding or neutralizing capabilities of the aptamers. For anti-thrombin aptamers, thrombin time (i.e., thrombin clotting time) will be used. In brief, 50 μL of release medium will be incubated for 2 min at 37° C. in 100 μL of fresh human plasma. 50 μL of thrombin containing 2 NIH U/mL in 25 mM CaCl₂) will be then added into the human plasma solution. Clotting time will be measured using a coagulometer. For anti-IL-12 aptamers, human & mouse IL-12 reporter HEK 293 cells (InvivoGen) will be used. In brief, 50,000 cells will be treated with the mixture of IL-12 (10 ng/mL) and aptamers (varied concentration) overnight. 20 μL of induced HEK-Blue IL-12 cell supernatant will be transferred to 180 μL of Quanti-Blue Solution. The SEAP levels will be determined at 630 nm using a spectrophotometer. Dose-response of HEK 293 cells to IL-12 will be acquired to determine effective concentration of IL-12 (i.e., EC50) with or without aptamers. The similar method and procedure will be applied to determine the function of anti-IL-10 aptamers by using IL-10 reporter HEK 293 cells. See FIGS. 7A-7C and 8A-8B.

### Results

Based on next generation sequencing and analysis of abundant sequences, top 50 candidates can be identified. After alignment and phylogenetic analysis, 10 to 12 candidates can be chosen for the evaluation of binding strength. The top 3 to 5 candidates are expected to bind target proteins with a K_{D} value at nanomolar or sub-nanomolar levels. The aptamer selected with the proposed method will bind to thrombin with a lower K_{D} value than those aptamers that were selected using the traditional membrane filtration method. With truncation, the length of the candidates can be shortened to 30 to 50 nucleotide long. The truncated aptamers can bind to target proteins with similar strength as the parent aptamers. Some of the truncated aptamers will not only bind, but also neutralize target proteins; others will bind but not neutralize target proteins. The selected aptamers can be used to develop biomaterials for regenerative medicine or immunotherapy applications.

After the bioinformatics analysis, 10±2 candidates for each target were selected for binding affinity analysis. FIGS. 7 and 8 show the comparative binding responses of each candidate to the respective target. Candidates IL10.49, 2.IL10.3, 2T.IL10.13, T.7 and IL12.35 were selected based on maximum response for further binding affinity analysis.

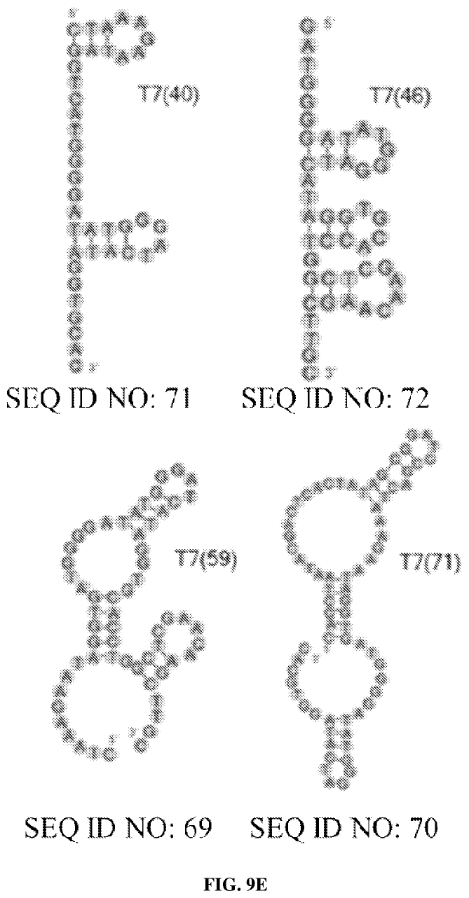

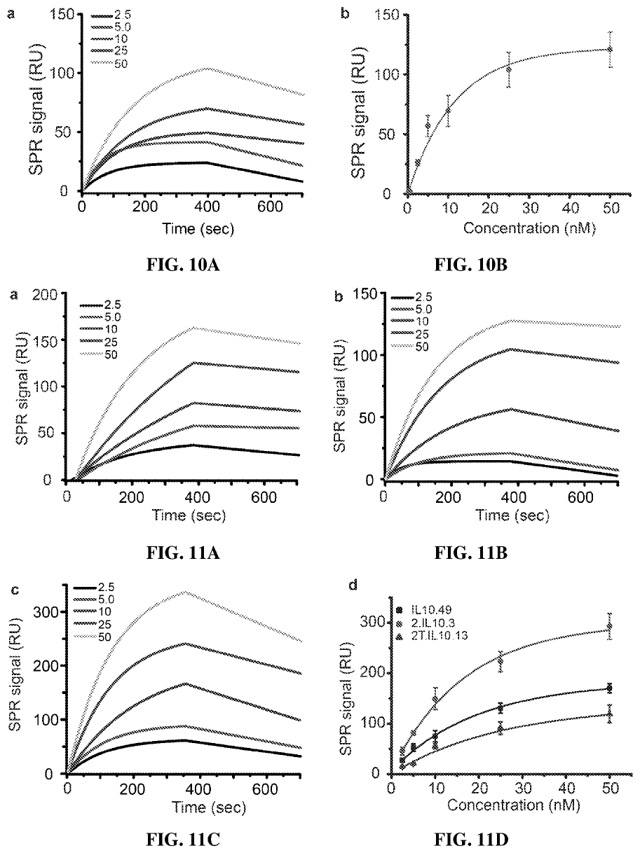

Thrombin protein was immobilized over 10% carboxylic-PEG SPR chip through EDC/NHS covalent chemistry. The prepared SPR chips were than utilized for binding affinity analysis with increasing concentration of selected aptamer. The binding of aptamer over the sensor chip led to increase in SPR angle, reaching the stabilization plateau within 6 min of association phase. After the binding, buffer solution was passed over the surface to remove loosely bound protein from the surface as dissociation phase (FIG. 9A). Calibration curve was plotted against the SPR max signal verses aptamer concentration (FIG. 9B). The binding affinity was calculated using the single ligand binding site equation in 1:1 interaction mode, A+B=AB, where A is injected aptamer and B is immobilized protein, AB is aptamer protein complex formed over the SPR chip. The formation of aptamer protein complex over the SPR chip is directly related to SPR response unit. The K_{D} value of aptamer was calculated using the equation RU=RUmax×C/KD+C, where C is the aptamer concentration. The determined KD value for thrombin T.7 aptamer to be 1.41±1.11 nM.

Truncation study was performed to shorten the T-7 aptamer. The purpose of this study is to compare the 90-mer thrombin aptamer (T-7) with previously reported (DNA 60-18) 29-mer aptamers {KD-0.5 nM, (Tasset et al., 1997)}. Thus, a truncation study was performed to make a fair comparison between similar-length aptamers. The truncation strategy relied on analyses of the secondary structure generated by mfold. Then the truncated sequences were experimentally verified with SPR (FIG. 9C, 9D). The result indicated the T.7 (45.1) exhibited excellent binding response compared to full-length and other truncated sequences. Later a comparison study was performed with previously reported standard aptamers (15 mer, 29 Mer) and truncated T.07 (45.1) aptamer (FIG. 9E, 9F). As depicted in the figure truncated T.7 aptamer showed a better response compared to the previously reported aptamer.

Mouse Interleukin-12 protein was immobilized over 10% carboxylic-PEG SPR chip through EDC/NHS covalent chemistry. The prepared SPR chips were then utilized for binding affinity analysis with increasing concentrations of IL12.35 aptamer. The binding of IL12.35 aptamer over the sensor chip led to an increase in SPR angle (FIG. 10A). The calibration curve was plotted against the SPR max signal versus aptamer concentration (FIG. 10B), and the sensor surface gets saturated after 25 nM. The binding affinity was discerned 9.72±2.75 nM by using the single ligand binding site equation in 1:1 interaction mode.

The binding affinity of selected aptamer candidates against human IL10 was evaluated using repeated HAS-two rounds or extended (2T) HAS time compared to a single round of HAS. The SPR study was performed with the best-selected aptamer from one round (IL10.49), 2 rounds (2rn.IL10.03), and extended time (2T. IL. 10.13) candidates (FIG. 11A-11C). The plotted calibration curve (FIG. 11D) indicated the binding affinity was similar in similar order for all three candidates. Therefore, one round of HAS is sufficient to produce aptamer with high binding affinity.

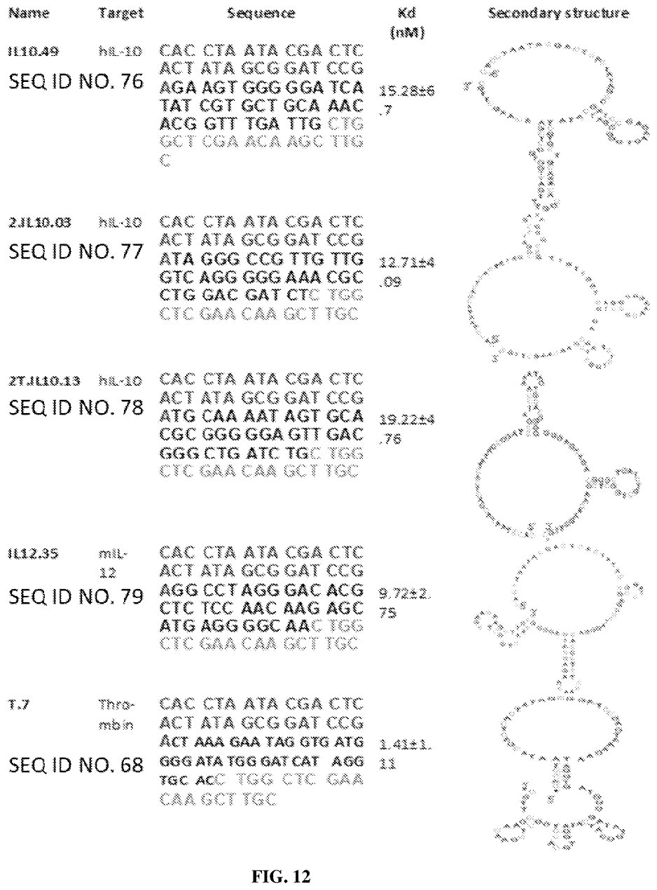

FIG. 12 shows the final selected aptamer sequences with respective secondary structure and measured binding affinity. The applicability of HAS was validated with proteins of different isoelectric point (pI) IL10-8.2±0.5, IL12-6.4±1.3 and Thrombin 6.8±1.0 and surface charge. Interestingly no direct relationship observed between the pI of protein and aptamer binding affinity. As evident from the study, HAS process could be used for all kinds of proteins in respective of their surface charge and pI. Sequences of respective proteins were identified in the UniProt database and isoelectric points were determined from the Compute pI/Mw tool at ExPASy.

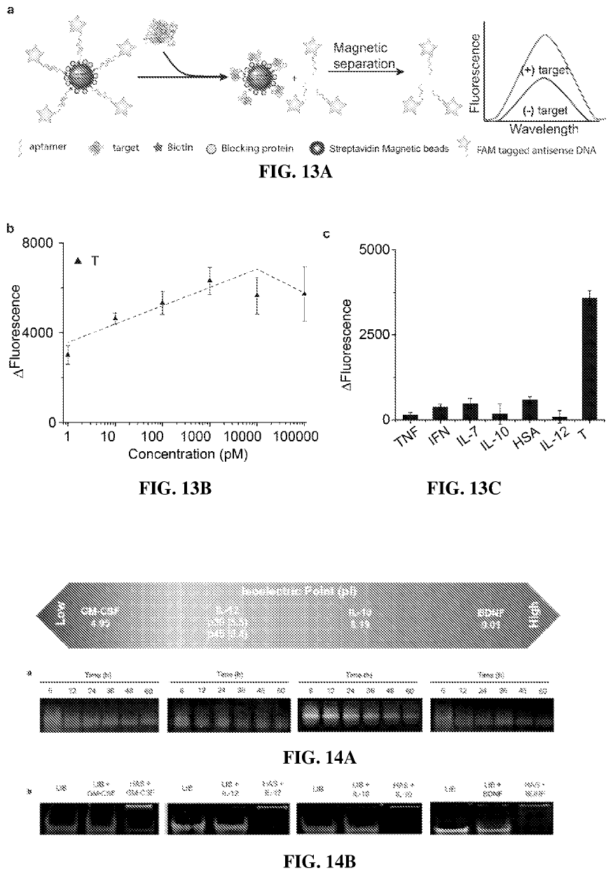

The practical application of the selected aptamer was validated through biosensing assay. A schematic representation of the aptamer-antisense displacement fluorescent-based assay was shown in FIG. 13A. The conformation-switching property of an aptamer in the presence of targets is the basic principle for this displacement assay. The sensor performance against the respective proteins was analyzed in a binding buffer with optimized parameters. A range of protein concentrations from 1 μM to 100 nM was studied for each selected aptamer. A linear response was observed from 1 μM to 1 nM for thrombin. The calculated limit of detection (LODs) was 0.2 μM for thrombin (FIG. 13B). The aptamer specificity against some analogous proteins was further investigated and no significant affinity was observed between the aptamer and nonspecific protein (FIG. 13C). It signifies the good selectivity of the selected aptamers against their specific target.

### REFERENCES

1. Ellington, A. D. & Szostak, J. W. In vitro selection of RNA molecules that bind specific ligands. Nature 346, 818-822 (1990).
2. Tuerk, C. & Gold, L. Systematic evolution of ligands by exponential enrichment: RNA ligands to bacteriophage T4 DNA polymerase. Science (80-.). 249, 505-510 (1990).
3. Lee, J. F., Stovall, G. M. & Ellington, A. D. Aptamer therapeutics advance. Curr. Opin. Chem. Biol. 10, 282-289 (2006).
4. Boltz, A. et al. Bi-specific aptamers mediating tumor cell lysis. J. Biol. Chem. 286, 21896-21905 (2011).
5. Abune, L., Davis, B. & Wang, Y. Aptamer-functionalized hydrogels: An emerging class of biomaterials for protein delivery, cell capture, regenerative medicine, and molecular biosensing. Wiley Interdiscip. Rev. Nanomedicine Nanobiotechnology 13, 1-25 (2021).
6. Song, S., Wang, L., Li, J., Fan, C. & Zhao, J. Aptamer-based biosensors. TrAC—Trends Anal. Chem. (2008). doi: 10.1016/j.trac.2007.12.004
7. Shi, P. & Wang, Y. Synthetic DNA for Cell-Surface Engineering. Angew. Chemie 133, 11684-11695 (2021).
8. He, F. et al. Aptamer-Based Targeted Drug Delivery Systems: Current Potential and Challenges. Curr. Med. Chem. 27, 2189-2219 (2018).
9. Zhang, Y. et al. Brain-targeted delivery of obidoxime, using aptamer-modified liposomes, for detoxification of organophosphorus compounds. J. Control. Release 329, 1117-1128 (2021).
10. Boshtam, M., Asgary, S., Kouhpayeh, S., Shariati, L. & Khanahmad, H. Aptamers Against Pro- and Anti-Inflammatory Cytokines: A Review. Inflammation 40, 340-349 (2017).
11. Rusconi, C. P. et al. Antidote-mediated control of an anticoagulant aptamer in vivo. Nat. Biotechnol. 22, 1423-1428 (2004).
12. Burmeister, P. E. et al. Direct in vitro selection of a 2′-O-methyl aptamer to VEGF. Chem. Biol. 12, 25-33 (2005).
13. Mahmoudpour, M. et al. Aptamer functionalized nanomaterials for biomedical applications: Recent advances and new horizons. Nano Today 39, 101177 (2021).
14. Gotrik, M. R., Feagin, T. A., Csordas, A. T., Nakamoto, M. A. & Soh, H. T.
   Advancements in Aptamer Discovery Technologies. Acc. Chem. Res. 49, 1903-1910 (2016).
15. Vant-Hull, B., Payano-Baez, A., Davis, R. H. & Gold, L. The mathematics of SELEX against complex targets. J. Mol. Biol. 278, 579-597 (1998).
16. Djordjevic, M. SELEX experiments: New prospects, applications and data analysis in inferring regulatory pathways. Biomol. Eng. 24, 179-189 (2007).
17. Mendonsa, S. D. & Bowser, M. T. In vitro selection of aptamers with affinity for neuropeptide Y using capillary electrophoresis. J. Am. Chem. Soc. 127, 9382-9383 (2005).
18. McNamara, J. O. et al. Cell type-specific delivery of siRNAs with aptamer-siRNA chimeras. Nat. Biotechnol. 24, 1005-1015 (2006).
19. Abune, L. & Wang, Y. Affinity Hydrogels for Protein Delivery. Trends Pharmacol. Sci. 42, 300-312 (2021).
20. Shi, P. et al. Polyvalent Display of Biomolecules on Live Cells. Angew. Chemie—Int. Ed. 57, 6800-6804 (2018).
21. Zhang, Z., Chen, N., Li, S., Battig, M. R. & Wang, Y. Programmable hydrogels for controlled cell catch and release using hybridized aptamers and complementary sequences. J. Am. Chem. Soc. 134, 15716-15719 (2012).
22. Battig, M. R., Soontornworajit, B. & Wang, Y. Programmable release of multiple protein drugs from aptamer-functionalized hydrogels via nucleic acid hybridization. J. Am. Chem. Soc. 134, 12410-12413 (2012).
23. Li, C. et al. Psoriasis-associated impairment of CCL27/CCR10-derived regulation leads to IL-17A/IL-22-producing skin T-cell overactivation. J. Allergy Clin. Immunol. 147, 759-763 (2021).
24. Shi, P., Zhao, N., Coyne, J. & Wang, Y. DNA-templated synthesis of biomimetic cell wall for nanoencapsulation and protection of mammalian cells. Nat. Commun. 10, 2223 (2019).
25. Singh, N. K., Chakma, B., Jain, P. & Goswami, P. Protein-Induced Fluorescence Enhancement Based Detection of Plasmodium falciparum Glutamate Dehydrogenase Using Carbon Dot Coupled Specific Aptamer. ACS Comb. Sci. 20, 350-357 (2018).
26. Singh, N. K., Arya, S. K., Estrela, P. & Goswami, P. Capacitive malaria aptasensor using Plasmodium falciparum glutamate dehydrogenase as target antigen in undiluted human serum. Biosens. Bioelectron. 117, 246-252 (2018).
27. Abune, L. et al. Macroporous hydrogels for stable sequestration and sustained release of vascular endothelial growth factor and basic fibroblast growth factor using nucleic acid aptamers. ACS Biomater. Sci. Eng. 5, 2382-2390 (2019).
28. Vulic, K. & Shoichet, M. S. Affinity-based drug delivery systems for tissue repair and regeneration. Biomacromolecules 15, 3867-3880 (2014).
29. Abune, L., Lee, K., Wang, Y., 2022. Development of a Biomimetic Extracellular Matrix with Functions of Protein Sequestration and Cell Attachment Using Dual Aptamer-Functionalized Hydrogels. ACS Biomater. Sci. Eng. 8, 1279-1289. https://doi.org/10.1021/acsbiomaterials.1c01544
30. Acinas, S. G., Sarma-Rupavtarm, R., Klepac-Ceraj, V., Polz, M. F., 2005. PCR-induced sequence artifacts and bias: insights from comparison of two 16S rRNA clone libraries constructed from the same sample. Appl. Environ. Microbiol. 71, 8966-8969. https://doi.org/10.1128/AEM.71.12.8966-8969.2005
31. Arnold, S., Pampalakis, G., Kantiotou, K., Silva, D., Cortez, C., Missailidis, S., Sotiropoulou, G., 2012. One round of SELEX for the generation of DNA aptamers directed against KLK6. Biol. Chem. 393, 343-353. https://doi.org/10.1515/hsz-2011-0253
32. Berezovski, M., Musheev, M., Drabovich, A., Krylov, S. N., 2006. Non-SELEX Selection of Aptamers. J. Am. Chem. Soc. 128, 1410-1411. https://doi.org/10.1021/ja056943j
33. Crivianu-Gaita, V., Thompson, M., 2016. Aptamers, antibody scFv, and antibody Fab′ fragments: An overview and comparison of three of the most versatile biosensor biorecognition elements. Biosens. Bioelectron. 85, 32-45. https://doi.org/10.1016/j.bios.2016.04.091
34. Ekblad, T., Bergström, G., Ederth, T., Conlan, S. L., Mutton, R., Clare, A. S., Wang, S., Liu, Y., Zhao, Q., D'Souza, F., Donnelly, G. T., Willemsen, P. R., Pettitt, M. E., Callow, M. E., Callow, J. A., Liedberg, B., 2008. Poly(ethylene glycol)-Containing Hydrogel Surfaces for Antifouling Applications in Marine and Freshwater Environments. Biomacromolecules 9, 2775-2783. https://doi.org/10.1021/bm800547m
35. Faccio, G., 2018. From Protein Features to Sensing Surfaces. Sensors 18, E1204. https://doi.org/10.3390/s18041204
36. Idili, A., Parolo, C., Alvarez-Diduk, R., Merkoçi, A., 2021. Rapid and Efficient Detection of the SARS-COV-2 Spike Protein Using an Electrochemical Aptamer-Based Sensor. ACS Sens. acssensors.1c01222. https://doi.org/10.1021/acssensors.1c01222
37. Kushwaha, A., Takamura, Y., Nishigaki, K., Biyani, M., 2019. Competitive non-SELEX for the selective and rapid enrichment of DNA aptamers and its use in electrochemical aptasensor. Sci. Rep. 9, 6642. https://doi.org/10.1038/s41598-019-43187-6
38. Lauridsen, L. H., Shamaileh, H. A., Edwards, S. L., Taran, E., Veedu, R. N., 2012. Rapid One-Step Selection Method for Generating Nucleic Acid Aptamers: Development of a DNA Aptamer against α-Bungarotoxin. PLOS ONE 7, e41702. https://doi.org/10.1371/journal.pone.0041702
39. Nitsche, A., Kurth, A., Dunkhorst, A., Pänke, O., Sielaff, H., Junge, W., Muth, D., Scheller, F., Stöcklein, W., Dahmen, C., Pauli, G., Kage, A., 2007. One-step selection of Vaccinia virus-binding DNA aptamers by MonoLEX. BMC Biotechnol. 7, 48. https://doi.org/10.1186/1472-6750-7-48
40. Plaxco, K. W., Soh, H. T., 2011. Switch-based biosensors: a new approach towards real-time, in vivo molecular detection. Trends Biotechnol. 29, 1-5. https://doi.org/10.1016/j.tibtech.2010.10.005
41. Pobanz, K., Lupták, A., 2016. Improving the odds: Influence of starting pools on in vitro selection outcomes. Methods San Diego Calif 106, 14-20. https://doi.org/10.1016/j.ymeth.2016.04.021
42. Wilson, R., Bourne, C., Chaudhuri, R. R., Gregory, R., Kenny, J., Cossins, A., 2014. Single-Step Selection of Bivalent Aptamers Validated by Comparison with SELEX Using High-Throughput Sequencing. PLOS ONE 9, e100572. https://doi.org/10.1371/journal.pone.0100572
43. Zakashansky, J. A., Imamura, A. H., Salgado, D. F., Romero Mercieca, H. C., Aguas, R. F. L., Lao, A. M., Pariser, J., Arroyo-Curras, N., Khine, M., 2021. Detection of the SARS-COV-2 spike protein in saliva with Shrinky-Dink@ electrodes. Anal. Methods Adv. Methods Appl. 13, 874-883. https://doi.org/10.1039/d1ay00041a
44. Zhuo, Z., Yu, Y., Wang, M., Li, J., Zhang, Z., Liu, J., Wu, X., Lu, A., Zhang, G., Zhang, B., 2017. Recent Advances in SELEX Technology and Aptamer Applications in Biomedicine. Int. J. Mol. Sci. 18, E2142. https://doi.org/10.3390/ijms18102142

### Example 2: High-Affinity One-Step Aptamer Selection Using a Non-Fouling Porous Hydrogel

Aptamers, commonly referred to as “chemical antibodies,” have been studied in various areas. However, the process of aptamer selection poses a significant challenge. In this study, we present a new method for aptamer enrichment and selection based on a diffusion-binding process in a three-dimensional non-fouling porous hydrogel with immobilized target proteins. Low-affinity aptamer candidates can be rapidly released from the hydrogel, while high-affinity candidates are restricted due to their strong binding to the immobilized protein targets. Consequently, a one-step enriched aptamer pool can be obtained within 60 hours and the enriched pool can strongly bind the protein targets. This enrichment is consistent across all five proteins with isoelectric points in the range from 5 to 9. With thrombin as a representative model, the anti-thrombin aptamer identified from an enriched aptamer pool has been found to have a binding affinity that is comparable to those identified over ten cycles of selection using traditional methods. Thus, this method is simple, fast, low-cost, and instrument-free, with a high probability of obtaining high-quality aptamers in one single step.

### Introduction

DNA and RNA oligonucleotides are widely used as molecular tools or building blocks in various applications, ranging from genome editing to molecular computing, due to their ability to recognize and hybridize with complementary sequences.^{[1-7]} Oligonucleotides selected from synthetic DNA or RNA libraries not only recognize complementary sequences, but also non-nucleic acid molecules, and these oligonucleotides are known as aptamers or “chemical antibodies”.^{[8-10]} Aptamers can be applied to most if not all areas where antibodies have been designed. Aptamers are chemically synthesized with little batch-to-batch variation and can be produced at a lower cost compared to antibodies. Additionally, aptamers exhibit high structural or functional stability under various storage and working conditions. Thus, aptamers have demonstrated great potential in a wide range of applications such as drug delivery, regenerative medicine, and biosensing.^{[11-17]} However, the rigorous selection of aptamers from libraries containing approximately 10¹⁴ to 10¹⁵ molecules presents a significant challenge.^{[18-20]}

Commonly used methods for aptamer selection use an iterative process of enrichment involving incubation, partition, and amplification. These methods typically require eight to fifteen cycles of enrichment, with each cycle reducing the number of aptamer candidates by approximately one order of magnitude.^{[18-20]} This process is associated with several challenges, including PCR amplification bias, ineffective partition, and even conformational changes in proteins or aptamer candidates being selected.^{[18-20]} Furthermore, most selection methods rely on two-dimensional selection matrices (e.g., nitrocellulose membrane). Aptamer candidates in the library can nonspecifically bind to these matrices for enrichment even though depletion steps are involved. On the other hand, once a high-affinity aptamer candidate reversibly dissociates from the target molecule and leaves the selection surface to the selection solution, it becomes difficult for this candidate to re-bind to target molecules during partition, causing the loss of suitable candidates. Thus, current methods for aptamer selection can be time-consuming, labor-intensive, and costly with no guaranteed success in obtaining high-affinity aptamers.^{[18]} To advance the process of aptamer selection, numerous alternative methods have been studied, including microbead-based selection, capillary electrophoresis-based selection, microfluidic-based selection, and others.^{[21-24]} While these methods have shown progress in improving the process of aptamer selection, common or specific challenges, such as reduced sequence diversity, still exist.

A simple enrichment and selection method that is fundamentally different from existing methods in concept is described. Its working principle is a coupled diffusion-binding process in a three-dimensional space that is non-fouling and highly permeable (FIG. 1). This space allows for free and fast diffusion of oligonucleotides without nonspecific binding or entrapment. Conversely, high-affinity aptamer candidates are restricted in their diffusion and release since this space is rationally designed to immobilize target molecules and the candidates strongly bind to the immobilized targets. Even if a high-affinity candidate dissociates from a target molecule, it will re-bind to the next target molecule down the pathway of diffusion in the three-dimensional space, rather than on a two-dimensional surface. This method is essentially equivalent to an instrument-free, automatic, iterative, and sequential incubation-partition process that does not suffer from nonspecific binding, PCR amplification bias, or reduced sequence diversity.

### Results

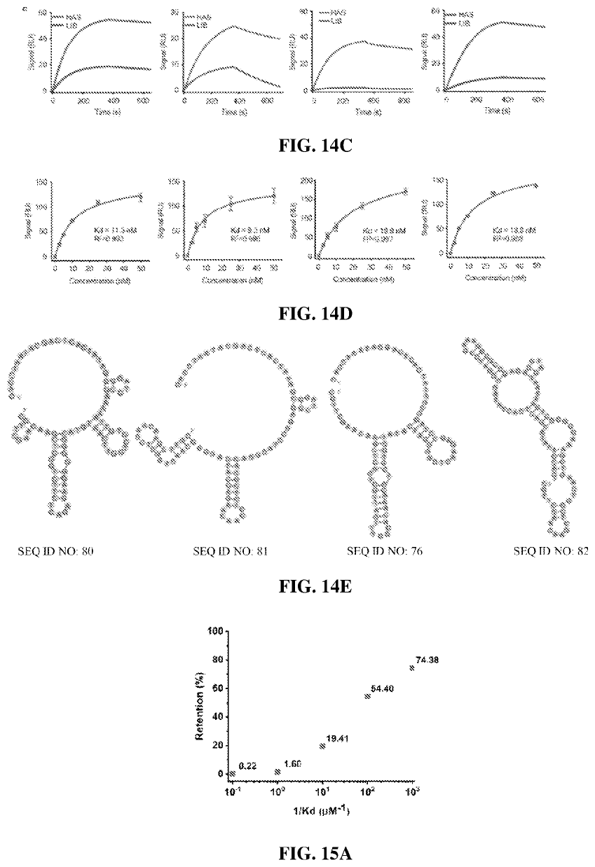

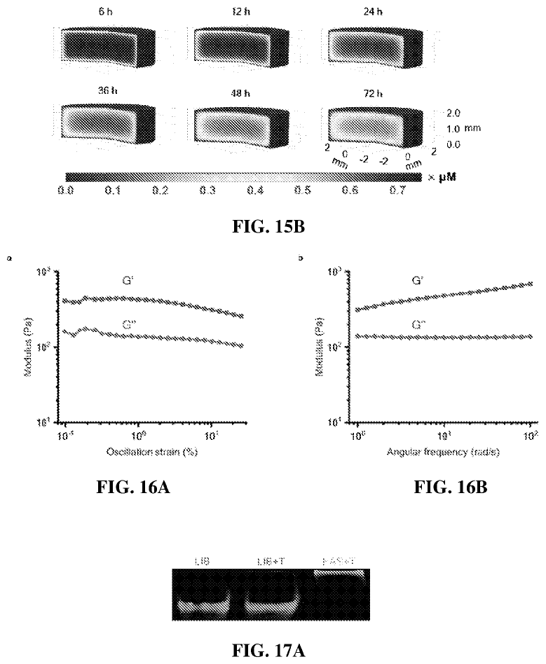

Modeling analysis. A diffusion-binding model was first used to evaluate the three-dimensional transport behavior of aptamers with different binding affinities as indicated by the Kd values ranging from 1 nM to 10 μM. The parameters used in this model are shown in Table 6. The modeling analysis showed that all aptamers were released from a defined porous space immobilized with target molecules (FIGS. 2A, and 14). The concentrations of the aptamers varied, with a higher concentration observed in the central region and a lower concentration on the periphery. However, different aptamers exhibited different distribution profiles spatially and retention rates temporarily (FIG. 2A, 14 and FIG. 15A-15B. For example, the retention of the low-affinity aptamer with a Kd of 1 μM at 24 hours was 14.1% whereas that of the high-affinity aptamer with a Kd of 1 nM was 84.9% (FIG. 2A). The retention of the aptamers with the Kd values ranging from 1 nM to 10 μM at 60 hours was 74.4%, 54.4%, 19.4%, 1.6%, and 0.2%, respectively (FIG. 15A-15B). The retention of the aptamer with a Kd of 1 nM was 46 times as much as that with a Kd of 1 μM. Therefore, the modeling analysis demonstrated that aptamers with different binding affinities have a highly different probability of retention in the porous space immobilized with target molecules due to the coupled diffusion-binding process.

TABLE 6
Parameters used in COMSOL simulation.

| Parameter | Value | Unit | Description |
|---|---|---|---|
| P | 1 | atm | Pressure |
| T | 298.15 | K | Temperature |
| EP* | 0.701 | 1 | Porosity of the hydrogel |
| kₒₙ, 1 nM | 1.0E+06 | /(s•M) | Forward rate constant of protein- |
| | | | aptamer binding (Kd = 1 nM) |
| k_{off}, 1 nM | 1.0E−03 | 1/s | Reverse rate constant of protein- |
| | | | aptamer binding (Kd = 1 nM) |
| kₒₙ, 10 nM | 3.3E+05 | /(s•M) | Forward rate constant of protein- |
| | | | aptamer binding (Kd = 10 nM) |
| k_{off}, 10 nM | 3.3E−03 | 1/s | Reverse rate constant of protein- |
| | | | aptamer binding (Kd = 10 nM) |
| kₒₙ, 100 nM | 1.0E+05 | /(s•M) | Forward rate constant of protein- |
| | | | aptamer binding (Kd = 100 nM) |
| k_{off}, 100 nM | 1.0E−02 | 1/s | Reverse rate constant of protein- |
| | | | aptamer binding (Kd = 100 nM) |
| kₒₙ, 1 μM | 3.3E+04 | /(s•M) | Forward rate constant of protein- |
| | | | aptamer binding (Kd = 1 μM) |
| k_{off}, 1 μM | 3.3E−02 | 1/s | Reverse rate constant of protein- |
| | | | aptamer binding (Kd = 1 μM) |
| kₒₙ, 10 μM | 1.0E+04 | /(s•M) | Forward rate constant of protein- |
| | | | aptamer binding (Kd = 10 μM) |
| k_{off}, 10 μM | 1.0E−01 | 1/s | Reverse rate constant of protein- |
| | | | aptamer binding (Kd = 10 μM) |
| Da* | 1.0E−07 | cm²/s | Diffusion coefficient of the |
| | | | aptamer in hydrogel |
| Cap_init* | 7.5E−07 | M | Initial concentration of aptamer- |
| | | | protein complexes |

*Hydrogel porosity was estimated by ImageJ based on the confocal microscopy images.
*Diffusion rate of proteins or protein-aptamer complexes were set as 0 because proteins were chemically immobilized in the hydrogel.
*It was assumed that aptamers were initially bound to proteins immobilized in the hydrogel.

Synthesis and characterization of macroporous polyethylene glycol (PEG) hydrogel. A major challenge in aptamer selection is nonspecific molecular binding to selection matrices.^{[20, 25]} PEG is the most well-studied non-fouling polymer. Methods for preparing PEG hydrogels are simple and well-established.^{[26-28]} Thus, macroporous PEG hydrogels were synthesized to experimentally simulate the non-fouling, porous space for aptamer selection. This method of using rationally designed “hydrogel for aptamer selection” is simply called HAS.

The porous PEG hydrogel was synthesized through cryogelation, a process that involves free radical polymerization of PEG monomers and crosslinkers at a cold temperature (FIG. 3A). Concurrently, ice crystals were formed in the hydrogel network, which created large pores in the hydrogel structure upon melting. To examine the pore structure, confocal laser scanning microscopy (CLSM) was used that does not require any specific sample pre-treatment such as lyophilization. A trace amount of fluorescein PEG acrylate was added to the polymerization solution, revealing the three-dimensional porous structure of the PEG hydrogel (FIG. 3B). Scanning electron microscopy images also confirmed the high porosity of the PEG hydrogel (FIG. 3C), showing that the average pore size of the dehydrated hydrogel was approximately 50 μm (FIG. 3D). Due to the presence of the porous structure, the PEG hydrogel could absorb a large amount of solution and swell to a high degree (FIG. 3E). The rheology data further indicate that the PEG hydrogel was highly soft with a storage modulus of 300 pa due to the presence of large pores (FIG. 16A-16B). Overall, these results demonstrate that cryogelation was an effective method to create a highly porous PEG hydrogel with the potential of being used as a selection matrix.

Protein immobilization in the macroporous hydrogel network. To enable protein immobilization in a three-dimensional space, acrylic acid was added to the pre-gelation solution. After polymerization, the PEG hydrogel network would be incorporated with carboxyl groups that can be easily activated for protein conjugation using the standard carbodiimide chemistry (FIG. 3F). To test the conjugation, fluorescein-labeled albumin was used as a protein model. After conjugation, the PEG hydrogel was washed and examined using the Maestro Imaging System. The PEG hydrogel without carboxyl groups was used as a control for comparison. The fluorescence images show that albumin was immobilized in the hydrogel network (FIG. 3G), which was confirmed by infrared spectrometry (FIG. 3H).

Next, thrombin was immobilized in the PEG hydrogel network and loaded a well-studied thrombin aptamer 60-18 (29) into the hydrogel to examine whether aptamers can be retained by immobilized proteins in a three-dimensional space that is highly permeable and non-fouling. This aptamer was acquired through eleven cycles of enrichment using the traditional nitrocellulose membrane filtration method and had a reported Kd of 0.5 nM^{[10]}. After loading the aptamer to the thrombin-immobilized PEG hydrogel, the release solutions were examined and imaged the entire hydrogel. The results show that the aptamer 60-18(29) was retained in the hydrogel with a much higher percentage than its low-affinity control aptamer (FIG. 2C), with retention percentages of 27% and 9% at 24 hours, respectively. The imaging analysis was consistent with the release examination (FIG. 2B). The data demonstrate that low-affinity aptamers can be rapidly released from the three-dimensional macroporous PEG hydrogel whereas high-affinity aptamers can be retained with a higher probability in the PEG hydrogel immobilized with target proteins. The experimental data and the modeling analysis (FIG. 2A) show the same trend of aptamer retention in a macroporous space.

Aptamer selection against thrombin immobilized in the macroporous PEG hydrogel. After these fundamental studies, it was examined whether HAS could be applied to identify aptamers against thrombin in one single step from a N40 aptamer library (Table 2 of Example 1). The library was loaded into the thrombin-immobilized PEG hydrogel after partially dehydrating the hydrogel. The dehydrated hydrogel recovered to its original shape immediately after loading the library. The library-loaded hydrogel was incubated in the selection buffer that was collected and replaced with a new buffer solution at pre-determined time points (FIGS. 4A, 4B). The collected solutions were analyzed using both gel electrophoresis and UV absorption.

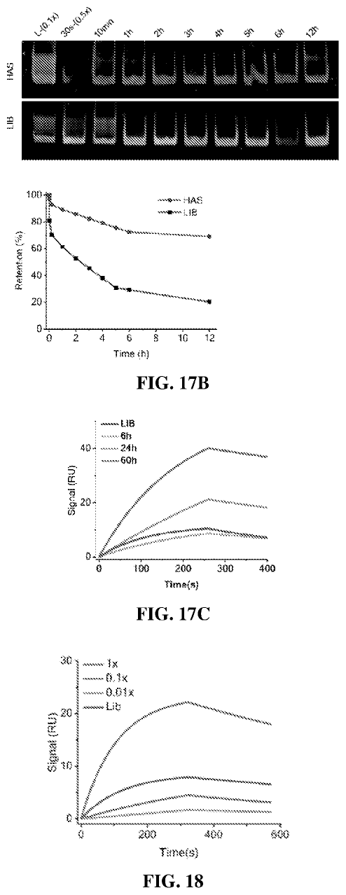

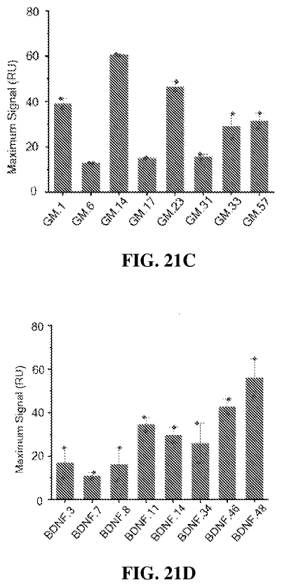

The target protein-immobilized (Thrombin, IL10, IL12, BDNF and GMCSF) hydrogel consisted of two components: the PEG-based selection matrix and thrombin or (i.e., other target molecule). Binding to the PEG matrix is considered nonspecific and contributes to background noise. Binding to thrombin, on the other hand, is considered specific and generates a positive signal. The library was rapidly released from the blank thrombin-free PEG hydrogel and its retention in this blank PEG hydrogel was close to 0% by 60 hours (FIG. 4A and FIG. 4B; FIGS. 21A and 21B), suggesting that the background noise due to nonspecific binding to the PEG matrix reached the minimal level. However, the concentration of the library retained in the thrombin-immobilized hydrogel was higher than that in the blank hydrogel at each time point of sample collection (FIG. 4A and FIG. 4B; FIGS. 21A and 21B). This comparison showed that the presence of thrombin in the PEG hydrogel affected the release of the library from the hydrogel, suggesting that the diffusion of potential aptamer candidates was retarded by the immobilized thrombin during the binding-diffusion process. As the background noise virtually reached 0% at 60 hours, 60 hours were chosen as the cut-off time point for the collection of enriched aptamer pools from the PEG hydrogel without further prolonging the release procedure. The aptamer pool collected at 60 hours exhibited a much stronger SPR signal than the original library (FIG. 17B). The SPR analysis was confirmed by the gel electrophoresis mobility shift assay and the aptamer release assay (FIG. 17A-17B).

To gain a better understanding of the aptamer selection process, aptamer pools eluted were collected at two earlier time points including 6 and 24 hours, and evaluated their ability to bind to thrombin using SPR analysis. The data show that the eluted aptamer pools at early time points did not exhibit strong binding signals (FIG. 17C), indicating that the aptamer pools collected at later time points had a stronger binding affinity for the immobilized thrombin than those collected at early time points. These results provide the evidence of successful aptamer enrichment through the one-step binding-diffusion process. Importantly, unlike traditional methods that require negative selection to eliminate potential non-specific binding of candidates to the selection matrix, the HAS method does not require negative selection because PEG, the selection matrix, is non-fouling. The thrombin concentration was also reduced by 10 and 100 times, and examined the capability of the collected aptamer pools in binding to thrombin. With the decrease of thrombin concentration, the collected aptamer pools did not exhibit strong binding to thrombin (FIG. 18). It suggests that it is important for immobilized thrombin to reach a threshold concentration in the macroporous hydrogel for effective aptamer selection.

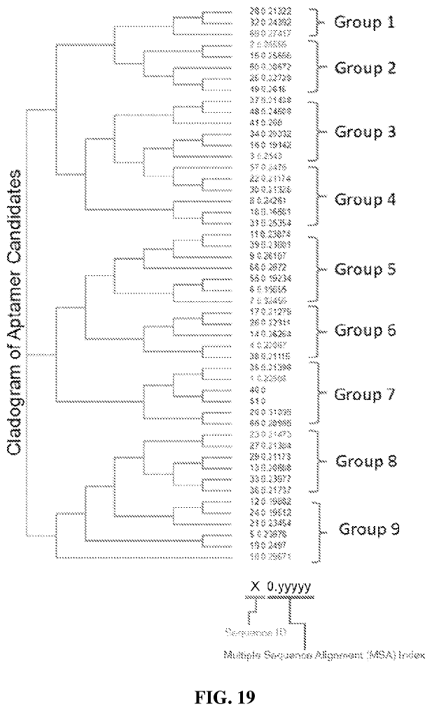

Anti-thrombin aptamer characterization. The aptamer pool collected from the hydrogel at 60 hours was analyzed using next-generation sequencing (FIG. 6). 5,905 sequences were acquired after filtering sequences inconsistent with the library in lengths and primer regions, selected the top 50 sequences representing 300 reads (Table 4) based on their abundance, and grouped them into 9 families (FIG. 19). One member from each family was chosen for analysis. One candidate showed a minimal binding signal, and the other eight candidates exhibited strong SPR binding signals (FIG. 8B and FIGS. 9C, 9D). Of these eight candidates, T.7 exhibited the strongest binding signal with a 1.5 nM Kd (FIG. 9A and FIG. 9B). A simple biosensor was used (FIG. 13A and FIG. 13B) to analyze the binding specificity of T.7. The comparison between thrombin and six control proteins showed that T.7 exhibited a high binding specificity (FIG. 13C). Thus, the data show that anti-thrombin aptamers were successfully discovered through one-step HAS.

The full-length T.7 aptamer, i.e., T.7 (90), has a multiple stem-loop secondary structure (FIG. 12). Based on this secondary structure, T.7 was truncated to generate six shorter aptamer sequences (Table 1). These truncated sequences were tested with SPR. T.7 (30) and T.7 (45) exhibited low binding strength whereas the other four truncated aptamers exhibited high binding affinities similar to the original T.7 aptamer (FIG. 9C and FIG. 9D). This observation is expected as a full-length aptamer contain both essential and non-essential nucleotides and aptamers will lose their binding affinities with essential nucleotides removed. [29-30] The four truncated aptamers exhibit different secondary structures (FIG. 9E).

TABLE 1
Sequences of thrombin aptamers.

| Name | SEQ ID NO. | Sequence (5′-3′) |
|---|---|---|
| T-7 (90) | 68 | CACCTAATACGACTCACTATAGCGGATCCGACTAAAGA |
| | | ATAGGTGATGGGGATATGGGATCATAGGTGCACCTGG |
| | | CTCGAACAAGCTTGC |
| T-7 (59) | 69 | CTAAAGAATAGGTGATGGGGATATGGGATCATAGGTG |
| | | CACCTGGCTCGAACAAGCTTGC |
| T-7 (71) | 70 | CACCTAATACGACTCACTATAGCGGATCCGACTAAAGA |
| | | ATAGGTGATGGGGATATGGGATCATAGGTGCAC |
| T-7 (40) | 71 | CTAAAGAATAGGTGATGGGGATATGGGATCATAGGTG |
| | | CAC |
| T-7 (46) | 72 | GATGGGGATATGGGATCATAGGTGCACCTGGCTCGAA |
| | | CAAGCTTGC |
| T-7 (30) | 73 | GATGGGGATATGGGATCATAGGTGCACCTG |
| T-7 (45) | 84 | CACCTAATACGACTCACTATAGCGGATCCGACTAAAGA |
| | | ATAGGTG |
| T-7 (45.1) | 74 | CACCTAATACGCTCACTATAGCGGATCCGACTAAAGAA |
| | | TAGGTG |
| T-7 (45.2) | 75 | GATATGGGATCATAGGTGCACCTGGCTCGAACAAGCTT |
| | | GC |

TABLE 4
Sequences and counts of top 50 candidates. N40 regions are shown.

| SEQ ID | | |
|---|---|---|
| NO. | Sequences Selected Candidates (5′-3′) | Counts |
| 107 | TTGGTCGAATAGCTCGAAGGTCCATACAGGGGTGTAAGCT | 15 |
| 108 | TGTAGGAGGGGACCGGGCTAGTTATATTTCACCTCTATCG | 12 |
| 109 | GCTCTTAATGTTAAGGGGTACAGTCCGAACAATTCTGGTG | 10 |
| 110 | GCAGTAGGGGCGTGTCCAAGTCTACTGAGCCGACAGATG | 9 |
| | G | |
| 111 | ATAGGGGGCTTCCACGTCCTGATAGAATAGAATAATGAGT | 8 |
| 112 | CGTCATACCCAAATATAAGTATACCAGAGAGGGGCCATTA | 8 |
| 113 | CTAAAGAATAGGTGATGGGGATATGGGATCATAGGTGCA | 8 |
| | C | |
| 114 | GATCTGGGGTCGTTATTTTACGGTAGAACGCATTTTGGTA | 8 |
| 115 | ACGCCCCGAATGTAGGTCCGTCGTGGTCTGGGGTTGTTTA | 8 |
| 116 | GGGGATACCCCTCTACAGCAATTGCGATTACACCCCTTGT | 8 |
| 117 | CCTTAAGCCACCGGGGGCATGACCTGTTTCTTTATTATTC | 8 |
| 118 | TTCTTTTGAATGTGCTGGGGTTACTCGAGTGGTATCTAT | 7 |
| 119 | CTCGGTATTTATCCATACTTGGGGTACGATCGTTTTAGTC | 7 |
| 120 | GGATTCCGGGGCTAGTGTAGGTTTCAGTTTCGTTACATGA | 7 |
| | C | |
| 121 | TGGGGAGTAAGCTGAGGCTGAACCTATCTTACCTGAGTCC | 7 |
| 122 | GGGGATTATGGTAAATGGCAGTACACTTCCCAGGTTTTCA | 7 |
| 123 | GTTCTACTCGACGGTATATAGTTCGTGGGGATGGTCGACA | 7 |
| 124 | AAATAACCGCCCCCGAGTGGGCCGTATTACAGGGGTAAGT | 7 |
| 125 | AGTTTCATTGCATCGTCCGGGGGTCGATACCACTCACAAG | 7 |
| 126 | CAGAGACCATGGCCCTTTATGCGGATCTGTTGCGGGGATC | 6 |
| 127 | TTTAGTAGCATCACATCGTTTTCCGGGGATGGGCACTACA | 6 |
| 128 | GTTATCGCGGGGAATACAGGGTGAAGCTAGCCCGCCGGA | 6 |
| 129 | GCCATTGGGATTAAATGCGTGGGGATCCGTTTTGATTGAG | 6 |
| 130 | ACTTGAATTATAACGGGGTCCTGTTGAGCAGATTCAACGT | 6 |
| 131 | TGTAAACGGGGATGTCTCTTCAGATGCAGGAAATGGGGAC | 6 |
| 132 | AATGTACCTCGGGGTAAAGTTAATTAGTGGGTTGTGTGTG | 6 |
| 133 | GTCCGTACTTAGCATAGTTGGGGCCCCCGTTCATATGGG | 6 |
| 134 | TTTTGGTGGGGAGAGTGTATTGTCCTGAGTCCTTGCAGCT | 6 |
| 135 | TCCCAGTTATTCAAGCTCGTTCTATTAACGTATTAGGGGG | 6 |
| 136 | AATATGCGCATGGAGATCACTGCGTGCCCGCGGGGGATCG | 5 |
| 137 | TAGTGGGGAATGTTAATGGAGTCAGAATGTAGTCTATGAG | 5 |
| 138 | TTGTCCGGACCGTTCCCTTGACGGGGTTACTTTAGCAAT | 5 |
| 139 | GCGGGGTGAATAAACAGGTGTTATCTTTGAACTGTAGGGT | 5 |
| 140 | GGGATTTAGGAACAAATCTGATATGGGGCCTCATACGGTT | 5 |
| 141 | GCTGAAGAGTAAGCCGCTCCGAAGGATTTAACGGGGTGA | 5 |
| | A | |
| 142 | GGGTGCTATACCGGCCGGACTGTGGGGTTCACTTCATGGT | 4 |
| 143 | TTGTTGCATTGCCTTACTAATTAATTGGCCGAGGGGGAGC | 4 |
| 144 | GGAGTGTAGGGATGTATAGGGGCGTCTGGGCCACGTGATT | 4 |
| 145 | AATTCATTGATAGGGGGGGTGCTTCACTGAGGGAGATGTT | 4 |
| 146 | CTAGACATTATAATAGGGACTTCATGTGAGTTAAGGATGG | 4 |
| 147 | ACGTCAGACGTGTAACTCACTGTATGTAAATGCGGAAGTC | 4 |
| 148 | AAGTTGAATTTGTAACTCCACGGAATTATCATTGTTCCGG | 4 |
| 149 | TAAAAATTACCATGCCCGTTCAATTCGGGCAAGGGACGTG | 3 |
| 150 | TAAAGGCACCCCACCTAATAAAATTGCCCCTTAGTACTAT | 3 |
| 151 | CTAGACATTATAATAGGGACTTCATGTGAGTTAAGGATGG | 3 |
| 152 | AGAATTATATATAAATGTAAATGTACGGTACTACGTCTGA | 3 |
| 153 | GCTCGGTGTTTATATGGAGTGACGCCACCGGGGACGGGAG | 3 |
| 154 | TAAGTAAACATGGGAGTTTGTAGCTCGAGAGACTCTATAA | 3 |
| 155 | TTACTTAGATCATGGTGATCGATGCGTCTCTTCGAACATG | 3 |
| 156 | CTGGCGGGGCACTCGGGAGAAAACGGAGAGCACTTAACT | 3 |
| | G | |

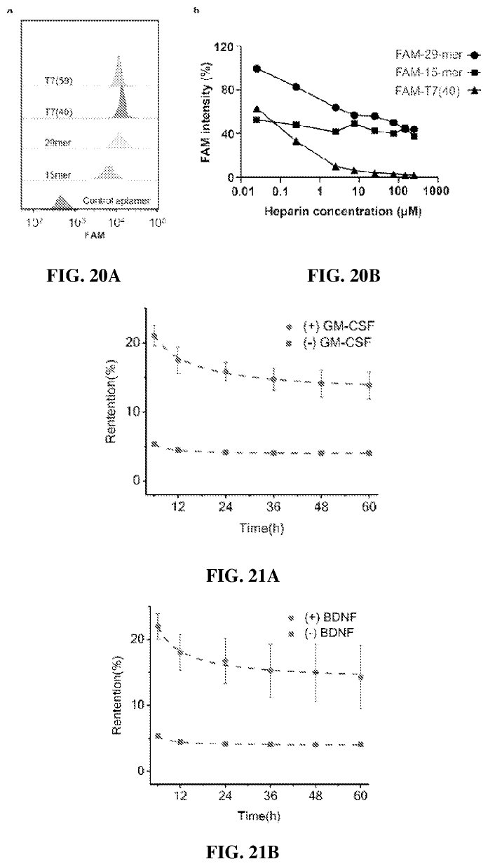

Thrombin was purposely chosen as the representative protein model for aptamer selection since the two well-characterized and widely used anti-thrombin aptamers, 15-mer and 60-18 (29),^{[10,31]} could be used as gold standards to compare the efficacy of the HAS method to traditional SELEX methods. Both 15-mer and 60-18 (29) were selected through over ten cycles of SELEX enrichment. The SPR analysis showed that T.7 (40) exhibited a binding affinity comparable to 15-mer and 60-18 (29) (FIG. 9C and FIG. 9D and FIG. 20B). Thus, the data show that HAS is effective in selecting high-affinity aptamers in one single step.

Aptamer selection against four additional protein targets with pI values ranging from 5 to 9. The majority of proteins have pI values in the range of 5 to 9.^{[32]} To faithfully show that HAS can be applied as a technology platform for other targets beyond thrombin, aptamers were selected against four additional proteins with pI values covering the range from 5 to 9. These proteins were granulocyte-macrophage colony-stimulating factor (GM-CSF), interleukin-12 (IL-12), IL-10, and brain derived neurotrophic factor (BDNF), which represent negatively, neutrally, and positively charged molecules. The aptamer selection against these four proteins was consistent with that against thrombin (FIG. 14A-14E and FIG. 21A-21D). The library was released more slowly from the protein-immobilized PEG hydrogel than the blank protein-free hydrogel. The one-step enriched aptamer pools showed stronger binding to the target proteins than the initial library. Aptamers with low Kd values were identified from the enriched pools. The successful selection of aptamers against a range of proteins with varying charges suggests that the HAS method is effective for discovering aptamers against diverse targets. The sequences and structures of aptamer candidates against these proteins are shown in Table 3.

TABLE 3
Aptamer candidate′s sequence.

| Aptamer | SEQ ID | |
|---|---|---|
| candidates | NO: | Sequence (5′-3′) without conserved region of library |
| IL10.58 | 2 | CATCTCTTGGTCAAGGTGAAGTTGCGCTAACTTCGCCAGA |
| IL10.11 | 3 | GGCGTCTTGTATAATACCATTCGCATTGTGCAAAATGGTC |
| IL10.08 | 4 | GATATCCTTTACCGCTGTCCACAATGCGGTATACGAATTAGT |
| IL10.49 | 5 | GAAGTGGGGGATCATATCGTGCTGCAAACACGGTTTGATTG |
| IL10.31 | 6 | GATAGTCGTATCGATCTGTAGAAACTTTCTGATACGTTCCGG |
| IL10.60 | 7 | GATAGTTCTCTGTGACTGTGTGAGACTCATCGCTGAGATCCG |
| IL10.9 | 8 | CTACACAAGTATGAGCCCTAGGCACAAACATTGATTTCTGCT |
| IL10.21 | 9 | CATTAAAGGATAGAGTATTGTGTTAGGCCACACACTATTC |
| IL10.38 | 10 | CCGATCTTACACAGAAGCAGGTCTCTGCTCTCCGGCAGTA |
| IL10.28 | 11 | CGTTGGGCGTCGCTGGACAGTGTTGGACCGACGGCTGGAC |
| IL 10.8 | 85 | GATATCCTTTACCGCTGTCCACAATGCGGTATACGAATTAGT |
| 2xT.IL10.30 | 12 | CCCGGGCTGGTGGGCTAGGAAGCAATTGGATCCCTGGCTA |
| 2xT.IL10.13 | 13 | TGCAAAATAGTGCACGCGGGGGAGTTGACGGGCTGATCTG |
| 2xT.IL10.57 | 14 | CCTACAACACCACAAGGCGTTGATGTAGTGAGGATTGTTA |
| 2xT.IL10.33 | 15 | TAGGGTGACCACAGGGGTGATAATCTATCGCCTCGAAAGT |
| 2xT.IL10.39 | 16 | CTTGGGACCGCTGTTAAGAAAAAAGTGTCGCAAGCGGGAA |
| 2xT.IL10.37 | 17 | AGGCGGGGGCTAGATCCGAAGGGAAGGACTGTAAGTCCCC |
| 2xT.IL10.14 | 18 | CGCCCTGGCTATCGTTAAAGCCGCTACGATGCAGAGTGCA |
| 2xT.IL10.27 | 19 | AAGATGTGCCGCAGGCCGAGATCCTAACTTCAGTCACTCA |
| 2xT.IL10.4 | 20 | GCACTCGAGCGGTCCGGGCCGCACTCTCGGCATTGCGCAA |
| 2xT.IL10.75 | 21 | ATGGTGTGTTGGACTACATAGACACCTCGAGGGTGAGGGG |
| 2rn.IL10.2 | 22 | CAACCCATGTATTACGGGTAACCGGATCAGAAACCCAGTG |
| 2rn.IL10.3 | 23 | TAGGGCCGTTGTTGGTCAGGGGGAAACGCCTGGACGATCT |
| 2rn.IL10.4 | 24 | TGGGCATACCACTAGGGGACGGATCGACAAAAGGGCGACG |
| 2rn.IL10.10 | 25 | GGTACCATGGGTACCTCATACTCAGCATTCAAGCCAGAAC |
| 2rm.IL10.13 | 26 | ATAGTGGCAGGGTACTCTTAGAGCGGATGGATGGAGGAGC |
| 2rn.IL10.14 | 27 | CACGCGGGAAGTCGGTGGTAGTGATCTATGGGCGCGACCG |
| 2rn.IL10.23 | 28 | ATACAACTGTCTTGTCAGGGTGTTCGGGGAGTTCTGAGGC |
| 2rn.IL10.25 | 29 | TTGGTTATGGCCAAAGTAACGCCTGAGCGTTGGTCGGTTA |
| 2rn.IL10.22 | 30 | CTATTTATGAGGAATATCCCAGACGGGACATCAGCATGCC |
| 2rn.IL10.5 | 31 | GCTCGCAGTGTATACGGTCTGCTAACAGCTCAGAGAGACA |
| IL12.36 | 32 | TGCAACGGGGCCGACCCGACATTTTGGCAGTGGGAGAACG |
| IL12.35 | 33 | GGCCTAGGGACACGCTCTCCAACAAGAGCATGAGGGGCAA |
| IL12.13 | 34 | AGGTAACAGTTACCAATCTTTTTGAGGATAGCATTTGGAA |
| IL12.28 | 35 | GGCATTTGGGATTTGGTTATGTTAACAAGGCATGGGCGGG |
| I112.19 | 36 | GACAAGTAATCATCGAGATTCGGATTTACGGTGGTGTGCA |
| IL12.20 | 37 | AGTTGGCCTAATGTAACGCCAAGGTGCCAAGTGTTTCTAT |
| IL12.10 | 38 | TCGGACTGGTTTGAGGGTTGCGGAACAGAGCCGTACCCCA |
| IL12.33 | 39 | TGTGATGTATATTAAGCGTGGGCCTCTATCGCACGCTTAC |
| IL12.5 | 40 | ACACGACATATCCACGTTAACCACATAATCCGCTGTGGTA |
| IL12.4 | 41 | CAAATTGGCACCTACTCGTAGAATAAGGTAATTGTGCTCT |
| IL12.3 | 42 | TACACTTGTACACGTAATCGTAGGGGGACGGTAACGGGTC |
| T.7 | 43 | CTAAAAGAATAGGTGATGGGGATATGGGATCATAGGTGCAC |
| T.10 | 44 | GGGGATACCCCTCTACAGCAATTGCGATTACACCCCTTGT |
| T.57 | 45 | GCTCGGTGTTTATATGGAGTGACGCCACCGGGGACGGGAG |
| T.69 | 46 | CTGGCGGGGCACTCGGGAGAAAACGGAGAGCACTTAACTG |
| T.1 | 47 | GTTATAAGTGACGTACCCTCAGAGCATATGCTTGGTGTGG |
| T.2 | 48 | TTAGCACTTTATTTAGGTTTTCGGGCCACGTGTGGTTG |
| T.3 | 49 | CGGCATATTGGTGTGTTGAATCGCTTAGATTAAGGAACCT |
| T.4 | 50 | GTGCTTCATTTACCTTCAGGTTGGGGATTGTGATAGAGC |
| T.23 | 51 | CAGCAGCACAGTGATGTTTGCCGACCAGGGTTATGAGGTT |
| GM.1 | 86 | TTCCAAAGGGCAGTCAAGTGTTTCTATCTCGTCCGGTATA |
| GM.6 | 87 | TACATCAATAAATGTTTGCATATTGATCTGTATGTTTAGA |
| GM.14 | 88 | AAAATTGTGCTGGTTCAATGTACGGGGGGAAGAGACTCTT |
| GM.17 | 89 | TTAATTTCAGGTTTAGCAGTACTTTGAAATTTTTAAGTTT |
| GM.23 | 90 | GACAGTCCAGTACGGAAATAATATTCGAACCATTTGTTAT |
| GM.31 | 91 | ATTAATAGAATGAGATGTATCTGGGTAAGTTTTTAAGTAT |
| GM.32 | 92 | ATCTATAACGAGAAATGTTAAGTCTTTTCTAATCACCTGA |
| GM.33 | 93 | TTGTGGAAAAATCATTTCAGATAATTTATTAATGTTGCAT |
| GM.41 | 94 | CCGAAGTATGTTTTCAATTGCACCTAGTTTTTTCTATCTT |
| GM.46 | 95 | TCTATTTTCTAGCAACTTTTATATGAGTTCTTTCTGTTGG |
| GM.57 | 96 | TGTTGGTTATTTTACGTCACAGACCGACTAGACATTGTCT |
| BDNF.3 | 97 | CTTATAGCTTGGGAAAAGTCCCTAATCAGATGATGTTTCA |
| BDNF.7 | 98 | ATGCGCCAATTTAGGTATAAAATGTAAAGAGTCATGATAG |
| BDNF.8 | 99 | TGTTCAATAAAGAATGTTATTGTATTAACAGATAGTTTCAA |
| BDNF.11 | 100 | GTAGTTAGATTTCATCTTTGCTTGTATGCTTATGTTATAA |
| BDNF.12 | 101 | GACTGATGTTACGCTGTGCGATCATGCGGACGCGCGGTTG |
| BDNF.14 | 102 | GTCGTGTTGGTATTTGGTTTGTATTGTGAATGCATTTATA |
| BDNF.34 | 103 | GGAGTGTATCTATAGTTCACTTGTTAGGACATTAGTGGCG |
| BDNF.46 | 104 | GTGATGAAAGATGAGTTAAGTATTTGAACAAGGTCTATAC |
| BDNF.48 | 105 | AAGGCGCCGAGACGTAGTAATAAGTCGGGGGAAAGGTGAA |
| BDNF.53 | 106 | ACCATTATATTCGAATGGACTCAACCAGCTCCAACACATA |

*IL10 or IL12 or T. XX is nomenclature coding where of IL10, or IL12 or T target protein, XX- selected candidate number), sequence highlighted in bold were selected for further validation study.

### Discussion

Aptamer selection systems consist of three main components including a target molecule, an aptamer library, and a selection matrix. The major variable determining optimal aptamer selection is the selection matrix that can take various forms, such as a membrane, particle, or device. As long as a selection matrix is used, one has to consider the binding of the library to the matrix. This library-matrix binding has to be minimized or avoided since it is a nonspecific binding that directly affects the efficiency of aptamer enrichment. Thus, the development of effective selection methods highly relies on choosing or establishing an appropriate selection matrix.

Although some matrix-free methods have been proposed, a truly matrix-free system with only a target molecule and an aptamer library may not be established in theory. For example, capillary electrophoresis-based aptamer selection, a promising method, requires a capillary that still acts as a selection matrix or support. The capillary may cause nonspecific binding and electrostatic interactions. Additionally, electrophoresis needs to be conducted under the influence of ionic current and electric field, which involves buffer solutions with low ionic strength that differ from physiological conditions and potentially affect the conformation and interactions of proteins and aptamers [33-35]. As a selection matrix has to be used, it is necessary to minimize or eliminate its interaction with the library. In the HAS method, a macroporous PEG hydrogel is used as the selection matrix since PEG is the most well-studied non-fouling molecule. The data clearly show that the library rapidly diffuses out of the macroporous protein-free PEG hydrogels, indicating that nonspecific interactions between the library and the matrix is minimal.

Two types of nonspecific binding need to be considered in aptamer selection processes. One is the nonspecific binding between the library and the selection matrix as discussed above. The other is the random or low-affinity binding of sequences to the immobilized target molecules. Therefore, apart from minimizing the nonspecific binding between the library and the matrix, it is important to eliminate sequences that bind nonspecifically to the target molecules. In the HAS method, this elimination is achieved through a coupled diffusion-binding process. Aptamer candidates exist either in a free state or in a bound state. Their concentrations in these states are determined by their binding affinities or dissociation constants. High-affinity candidates have a lower concentration in the free state, and vice versa. As diffusion is driven by the concentration gradient of free molecules, the apparent diffusion or release of high-affinity candidates is slower than that of low-affinity candidates. This difference in diffusion or release between high-affinity and low-affinity aptamer candidates within the three-dimensional non-fouling space leads to their automatic separation. However, it is important to note that, theoretically, the concentration of nonspecific or low-affinity candidates cannot reach zero due to the inherent molecular transport characteristics. An enrichment pool will still contain low-affinity candidates, which has been demonstrated through both modeling and experimental results.

Aptamers were selected against five proteins with the pI values ranging from 5 to 9 to avoid the potential bias of selecting aptamers against positively charged proteins that are typically considered as “aptamer-friendly” or “selection-friendly” molecules in the field. Among these five proteins, GM-CSF is the most negatively charged molecule with the lowest pI value. However, specific advantages or disadvantages in the enrichment of the aptamer pool against GM-CSF were not observed in comparison to the other four neutrally or positively charged proteins. Notably, while aptamer selection against proteins was studied, in principle, HAS can be applied to the selection of aptamers against other molecules or even living cells if they can be chemically immobilized or physically entrapped in the macroporous PEG hydrogel under mild conditions. However, aptamer selection is accomplished in 60 hours under the current working conditions that may decrease cell viability. Thus, if HAS can be optimized or integrated with other methods for a shorter duration of aptamer selection, HAS may be applied to select aptamers against targets beyond proteins. Beyond its application in aptamer selection, the HAS method holds promising potential for screening other types of drugs or ligands, such as bacteriophages. Future effort can be made to explore these potentials.

It is known that while the theoretical diversity of a random N-40 aptamer library should be 4⁴⁰ (i.e., ~10²⁴), the actual diversity of a working library is ten orders of magnitude lower with 10¹⁴-10¹⁵ candidates. Despite the decrease, the library size remains huge, and in theory, there should only be one copy of each sequence. In fact, the real library contains multiple identical sequences due to sequence biases arising from chemical synthesis and PCR amplification^{[36-38]} Thus, while 50 aptamer candidates were prioritized based on abundance hits in this work, it is important to acknowledge that this prioritization is not perfect as biases or artifacts cannot be fully ruled out. As a result, the selection of the 50 sequences was actually a random pick, similar to the process of selecting ~30 to 100 colonies after the final cycle of enrichment in traditional SELEX methods. Therefore, the development of methods for picking candidates from an enriched aptamer pool deserves further investigation. In fact, while on one-step HAS was focused in this work, repeating HAS would lead to the acquisition of two sets of enrichment pools. By comparing the sequences from these two sets, one can prioritize aptamer candidates using FASTAptameR 2.0,^{[39]} which will be explored in the near future.

In sum, HAS is simple, fast, low-cost, and instrument-free for the enrichment of aptamer pools and the selection of aptamers. HAS holds great potential to promote the development of various areas ranging from drug development to clinical diagnosis.

### Materials and Methods

### Materials

Primers, ssDNA library and aptamers were obtained from Integrated DNA technologies (Coralville, IA). The aptamer library and primer sequences are reported in Table 2 of Example 1. Recombinant carrier free proteins, human Interleukin-10 (IL-10), murine interleukin-12 (IL-12), Brain-derived neurotropic factor (BDNF), and granulocyte-macrophage colony-stimulating factor (GM-CSF) expressed in Spodoptera frugiperda (SF 21) insect cells were procured from R&D systems (Minneapolis, MN). Thrombin (T) from human plasma, poly(ethylene glycol) monomethyl ether monoacrylate (PEGMA, Mn 480), polyethyl glycol diacrylate (PEGDA, Mn 750), N-hydroxysuccinimide (NHS), N-(3-Dimethylaminopropyl)-N′-ethylcarbodiimide (EDC), ethanolamine, morpholine ethane sulfonic acid (MES), triton-X, calcium chloride (CaCl₂)), sodium chloride (NaCl), magnesium chloride (MgCl₂), tetramethylethylenediamine (TEMED), ammonium persulphate (APS), acrylic acid, fluorescein o-acrylate and Amicon Ultra 3-10 kDa cut-off filter were all procured from Sigma Aldrich (St. Louis, MO). Agarose, Zeba desalting column (7 kDa), GeneJet PCR purification kit, DNA gel extraction kit, SYBR gold DNA dye and Tris-borate-EDTA buffer (10×) were purchased from Thermo-Fisher Scientific (Waltham, MA)). Q5 high fidelity DNA polymerase 2× master mix was obtained from New England Biolabs (Ipswich, MA). Streptavidin-coated magnetic beads (4 μm diameter) and streptavidin polystyrene micron particles (MPs) were procured from Spherotech (Lake Forest, IL).

### Simulation of DNA Diffusion and Retention in Hydrogel

COMSOL Multiphysics v. 6.0 was used to simulate DNA diffusion from hydrogel. ‘Transport of Diluted Species in Porous Media’ was applied in the model. A hydrogel disc with a diameter of 6 mm and height of 2 mm was constructed. The initial concentration of DNA in the release medium was assumed to be zero. The aptamers were initially bound to the protein immobilized in the porous hydrogel. Other parameters used for the model are summarized in Table S1. After all the parameters were input into the model, a simulation was executed over 60 h and data were collected at 12 h increments.

### Synthesis of Porous PEG Hydrogel

PEG hydrogels were synthesized using a method previously reported in literature with slight modifications. Briefly, a petri dish (35 mm diameter) was coated with 2 mL of 4% (w/v) Pluronic F-127 surfactant for 2 min and air-dried after the surfactant removal. Meanwhile, a mixture of 2 mL of 10% (v/v) PEGMA and 0.2% (v/v) PEGDA was prepared and supplemented with 0.2% (v/v) TEMED, 4% (w/v) APS and 1% (v/v) acrylic acid in 1×PBS of pH 7.3 and kept on ice. The pH of the polymerization solution was adjusted with 1 M NaOH before APS and TEMED were added. After the polymerization solution was mixed, it was transferred to the petri dish and allowed to polymerize at −20° C. for 12 hours. The hydrogel was incubated at room temperature for the removal of ice crystals and washed thoroughly with DI H₂O to remove any unreacted precursors. Disk-like specimens (6 mm in diameter, 2.0 mm in thickness and calculated volume is 56.5 mm³) were obtained through a biopsy punch.

### Immobilization of Protein in Porous Hydrogel

Carboxyl groups were incorporated into the PEG hydrogel through the addition of acrylic acid in the polymerization solution. Carboxyl groups were activated using EDC/NHS for covalent immobilization of protein via the formation of amide bonds. In detail, the hydrogel was first dehydrated by blotting the gel with Kimwipes. Then the gel was incubated in a mixture of freshly prepared 30 mM EDC and 20 mM NHS in 300 μL 10 mM MES buffer of pH 5.0 for 1 h at 4° C. After washing, the activated hydrogel was incubated with 1 μg of protein in 1×PBS (300 μL) at room temperature for 2 h. The protein-conjugated PEG hydrogel was washed for three times with washing buffer (1×PBS, 0.2% Triton X, 300 mM NaCl) under shaking to remove loosely/physically bound proteins and stored at 4° C. until used. FITC tagged BSA was used for the examination and demonstration of the successful protein conjugation. The conjugation efficiency of the protein was determined by quantification of the fluorescent signal throughout the hydrogel.

### Hydrogel Characterization

The PEG hydrogel was examined under the scanning electron microscope (SEM) (Sigma VP-FESEM, Zeiss), and confocal scanning microscope (Fluoview 3000, Olympus). For SEM imaging, the lyophilized hydrogel was coated with iridium prior to imaging. The pore sizes were determined from SEM images using ImageJ. For confocal imaging of the hydrogel, a fluorescent hydrogel was prepared by adding (0.04 wt % fluorescein o-acrylate) during the hydrogel synthesis using the protocol as mentioned previously. After the hydrogel was washed thoroughly to remove monomers and mounted on the coverslip, images were captured at 10× magnification. The swelling ratio of the hydrogel was calculated by dividing the mass of the fully swollen hydrogel by the mass of the dried hydrogel. FTIR spectra were collected using lyophilized hydrogel on a vertex 70 spectrometer (Brucker optics, Billeric, MA). A spectrum scanning was performed from 2000 to 500 cm-1 and absorbance was calculated by referencing the clear bare diamond crystal. Rheological characterizations were performed by TA-instrument Discovery HR-2 rheometer. Each experiment was carried with a 20 mm diameter parallel plate and 200 μm gap at room temperature. Typically, frequency sweeps were tested from 0.1 to 100 rad/s at 0.1% strain and amplitude sweeps were tested from 0.1% to 30% strain at 1 rad/s.

### Aptamer Selection

5 nmole of commercially obtained ssDNA aptamer library (5′-CACCTAATACGACTCACTATAGCG GATCCGA-N40-CTGGCTCGAACAAGCTTGC-3′) was dissolved in binding buffer (BB-1×PBS, 5 mM MgCl₂, 1 mM CaCl₂ and 2.5 mM KCl), denatured at 95° C. for 5 min, and cooled at room temperature for 10 min. Library was loaded onto the HAS by partially dehydrating the plug and allowing passive uptake of library in aqueous solution (See Supplemental Video). After the aptamer library was loaded into the porous PEG hydrogel, the gel was incubated in 400 μL BB at 4° C. on shaker for diffusion of loosely bound ssDNA from the hydrogel. The binding buffer was collected and replaced with fresh binding buffer at intervals of 12 h and this step was repeated up to 60 hours. After 60 hours, the hydrogel was treated with 400 μL of 1M NaCl for overnight to elute aptamer candidates from the PEG hydrogel. The eluted ssDNA sequences were desalted with Zeba desalting column and concentrated over the speed vac concentrator until approximately ~50-100 μL solution was obtained. The concentrated ssDNA sequences were amplified by PCR using the primers with following conditions: initial denaturation 98° C. for 2 mins, followed by 25 cycles of 98° C.-10 s, 68° C.-12 s, 72° C.-5 s and final extension at 72° C. for 5 min (Table 5). The sequence length and purity of PCR products were subsequently checked by running the samples on 2.0% agarose gel for 45 min at 90 V in 1×TBE. The target band was cut, and DNA band was extracted with GeneJet extraction kit by following the manufacturer's protocol and the purified product was used for sequencing.

TABLE 5

| | PCR reaction parameters. | | | |
|---|---|---|---|---|
| Reagents | | Volume (μL) | | |
| Q5 high fidelity | | 25 | | |
| 2x master mix | | | | |
| Forward primer | | 1 | | |
| (10 pmole/μL) | | | | |
| Reverse primer | | 1 | | |
| (10 pmole/μL) | | | | |
| Template (HAS- | | 20 | | |
| enriched product) | | | | |
| Nuclease free | | Adjust to 50 | | |
| water | | | | |
| | Temp- | | | |
| | erature | Time | | |
| PCR stage | (° C.) | (sec) | | Cyce |
| Initial denaturation | 98 | 30 | | 1 |
| Denaturation | 98 | 10 | [symbol] | |
| Annealing | 68 | 12 | | 25 |
| Extension | 72 | 05 | | |
| Final extension | 72 | 300 | | 1 |
| Storage | 4 | ∞ | | |

### NGS and Bioinformatics Analysis

Gel-purified DNAs were sequenced using Illumina next generation sequencing platform with distinct prespecified partial Illumina barcode and sequenced from the Azenta life sciences, USA. The raw data obtained in fastq format was imported into Galaxy web server for analysis by using standard methodology [41]. An overall evaluation of the data and general statistics were obtained using the “FASTQC and Trimmometric” tool using sliding window operation. The format of the data was then converted to FASTA using the “FASTQ to FASTA converter”. The length of the sequences was constrained to library length (80-100 bp) using the “Filter sequences by length” tool to remove amplification and sequencing artifacts from the data sets. Unique sequences were then identified using the “Collapse” tool. The topmost abundant sequences were identified after trimming, QC, collapsing, analyzed, and characterized. Finally, the selected top 50 candidates based on abundance hit were identified as potential binders with target protein. Notably, in theory, these sequences should have single copies rather than multiple ones. Prioritizing these sequences for aptamer identification is not perfect as it is largely similar to the random pick of colonies after the final round of aptamer selection in traditional methods. Multiple sequence alignment of these sequences using Multalin (http://multalin.toulouse.inra.fr/multalin/)/Clustal omega (https://www.ebi.ac.uk/Tools/msa/clustalo/) was finally performed to identify sequence families.^{[42-43]} Based on this information, single candidates from each family were chosen based on lowest Gibbs energy as calculated by mFold (http://www.unafold.org/mfold/applications/dna-folding-form.php) for further bioinformatics analysis. Final selected sequences were sent for synthesis for further analysis^{[44]}.

### Strand Separation of PCR Amplified Product

For the DNA strand separation, the eluted DNA product was PCR amplified with 3′ biotin tagged reverse primer. The PCR amplified product was incubated with 25 μL streptavidin coated magnetic beads in binding buffer at room temperature for 30 min. The conjugated beads were then washed with binding buffer and ssDNA strands were separated from biotinylated strands using 100 μL of 100 mM NaOH. The pH of separated strand was adjusted to pH 7.3±0.5 with 10.5 μL of 1M KH₂PO₄ and quantified with nanodrop after the background substruction with respective buffer.

### Electrophoretic Mobility Shift Assay (EMSA)

Protein at various concentrations and 250 nM of separated ssDNA or aptamer were mixed in the binding buffer (25 μL) and incubated at room temperature for 30 min. The protein-ssDNA complexes were loaded in a 10% PAGE-TBE gel and separated at 90 V for 2 hr at 4° C. After electrophoresis, the gel was stained with SYBR gold for 15 min for visualization.

### Surface Plasmon Resonance (SPR) Analysis

Different methods have been used to examine the same aptamer, and the acquired K_{d} values from different labs can be several orders of magnitude difference and hard to compare. Surface plasmon resonance (SPR) is a common method for determining the K_{d} value of aptamers. Thus, in this study, the same technique (i.e., SPR) was used to measure and compare previously or newly selected aptamers, enriched aptamer pools, or DNA libraries against immobilized proteins (IL10, IL12, BDNF, GMCSF and Thrombin) using Reichert SPR 7000DC (USA) for fair comparison. Proteins were conjugated onto the carboxyl group coated sensor chip. Prior to the protein injection, the left channel of the sensor chip was activated by the mixture of 400 mM EDC and 200 mm NHS in 10 mM (pH 5.5) MES buffer at flow rate of 20 μL/min for 15 min. Later, the left channel was immobilized with protein (10 μg/mL in 10 mM MES buffer, pH 5.5) for 20 min at 10 μL/min, followed by blocking with 1M ethanol amine. Before the measurement, the system was equilibrated with running buffer until stable sensorgram signal was observed. The running buffer was a binding buffer supplemented with 0.05% of Tween-20. For the measurement, the solution of analytes was passed over the SPR chip at 20 μL/min for association followed by a dissociation phase with binding buffer. The SPR chip was regenerated by washing with 30 mM NaOH at 100 μL/min for 1 min. Data was collected using SPR Autolink Software (Reichert Technologies) and analyzed using Scrubber and Origin software. The K_{d} value of the aptamer was calculated by plotting a graph of maximum SPR response versus the concentration of proteins and fitted in single ligand binding model of Scrubber 2.0 software.

### Binding and Competition Assay

The binding and competition assay was performed using 5 μm thrombin-coated microparticles (MPs). To prepare the thrombin-coated MPs, 0.1 mg (20 μL 0.5% w/v) streptavidin modified MPs were treated with 1 μg biotinylated thrombin at room temperature. After 1 h incubation, the MPs were washed twice with DPBS to remove the excessive biotinylated thrombin. For the binding assay, 0.1 mg thrombin coated MPs were resuspended in 400 μL binding buffer, and then 2.5 μM Fam labeled aptamer (T7 (59), T7 (40) 29mer, 15mer or scrambled aptamer KK) was added into the solution (Table 2 of Example 1). The samples were incubated at room temperature for 1 hour followed by gentle washing with binding buffer (same as the aptamer selection buffer) twice. The microparticles were examined using flow cytometer. For the competition assay, the Fam labeled aptamer was fixed at 2.5 μM. The heparin solution with varied concentrations (0, 0.025, 0.25, 2.5, 7.5, 25, 75, 150, 250 μM) was added to compete against the aptamers. The final volume was 400 μL. After 1 h incubation at room temperature, all samples were washed with binding buffer and measured using flow cytometry.

### Antisense Strand Displacement Assay for Protein Detection

An equimolar (10 μM) mixture of 6-FAM (6-Carboxyfluorescein) modified aptamer (T.7) and biotinylated antisense strand in binding buffer was heated at 90° C. for 5 min and cooled at room temperature for 30 min. The unhybridized antisense strand was removed by filtration through a 10 kDa cutoff filter. Streptavidin coated magnetic bead (MB) was incubated with 10 μL of the hybridized aptamer at room temperature for 1 h under shaking and then washed with binding buffer to remove unbound aptamers. The MB surface was blocked by incubation with 0.1% BSA for 15 min and washed with buffer. The functionalized MB was suspended in buffer and stored at 4° C. For protein analysis, 200 μL of functionalized MB was incubated with the solution of protein at room temperature. After 30 min incubation, the supernatant was collected for fluorescence analysis (excitation at 2493 nm and emission at 2517 nm) using Tecan M200 pro. Binding specificity was evaluated with the same method.

### Aptamer Truncation

A standard aptamer structure consists of conserved forward primer binding region, reverse primer binding region, and in between 40 N random region. These primer regions were sequentially deleted and their effect on binding was evaluated with SPR. Truncation was further performed based on the analysis of the secondary structure derived from M-fold.

### Statistics and Reproducibility

Synthesis and characterization of the hydrogel in FIGS. 15A-15K. B and c were conducted in triplicate independently with similar results. Experiments in FIGS. 17A-17B were repeated twice independently with similar results. Experiments in FIGS. 20A and 20B were repeated twice independently with similar results.

### REFERENCES

[1] Cong, L., Ran, F., Cox, D. G., Lin, S., Barretto, R. P. J., Habib, N., Hsu, P. D., Wu, X., Jiang, W., Marraffini, L. A., & Zhang, F. Multiplex Genome Engineering Using CRISPR/Cas Systems. Science 339, 819-823 (2013).
[2] Jinek, M., Chylinski, K., Fonfara, I., Hauer, M. H., Doudna, J. A., & Charpentier, E. A Programmable Dual-RNA-Guided DNA Endonuclease in Adaptive Bacterial Immunity. Science 337, 816-821 (2012).
[3] Fire, A., Xu, S., Montgomery, M. W., Kostas, S. A., Driver, S. E., & Mello, C. C. Potent and specific genetic interference by double-stranded RNA in Caenorhabditis elegans. Nature 391, 806-811 (1998).
[4] Seeman, N. C., & Sleiman, H. F. DNA nanotechnology. Nature Reviews Materials 3 (2017).
[5] Rothemund, P. W. K. Folding DNA to create nanoscale shapes and patterns. Nature 440, 297-302 (2006).
[6] Benenson, Y., Adar, R., Paz-Elizur, T., Livneh, Z., & Shapiro, E. DNA molecule provides a computing machine with both data and fuel. Proceedings of the National Academy of Sciences of the United States of America 100, 2191-2196 (2003).
[7] Li, Y., Tseng, Y. D., Kwon, S. O., D'Espaux, L., Bunch, J. S., McEuen, P. L., & Luo, D. Controlled assembly of dendrimer-like DNA. Nature Materials 3, 38-42 (2004).
[8] Ellington, A. D., & Szostak, J. W. In vitro selection of RNA molecules that bind specific ligands. Nature 346, 818-822 (1990).
[9] Tuerk, C., & Gold, L. Systematic Evolution of Ligands by Exponential Enrichment: RNA Ligands to Bacteriophage T4 DNA Polymerase. Science 249, 505-510 (1990).
[10] Tasset, D., Kubik, M. F., & Steiner, W. Oligonucleotide inhibitors of human thrombin that bind distinct epitopes. Journal of Molecular Biology 272, 688-698 (1997).
[11] Boshtam, M., Asgary, S., Kouhpayeh, S., Shariati, L., & Khanahmad, H. Aptamers Against Pro- and Anti-Inflammatory Cytokines: A Review. Inflammation 40, 340-349 (2017).
[12] Famulok, M., & Mayer, G. Aptamers and SELEX in Chemistry & Biology. Chemistry & Biology 21, 1055-1058 (2014).
[13] Rusconi, C. P., Scardino, E., Layzer, J. M., Pitoc, G. A., Ortel, T. L., Monroe, D. M., & Sullenger, B. A. RNA aptamers as reversible antagonists of coagulation factor IXa. Nature 419, 90-94 (2002).
[14] Farokhzad, O. C., Cheng, J., Teply, B. A., Sherifi, I., Jon, S., Kantoff, P. W., Richie, J. P., & Langer, R. Targeted nanoparticle-aptamer bioconjugates for cancer chemotherapy in vivo. Proceedings of the National Academy of Sciences 103, 6315-6320 (2006).
[15] Liu, J., & Lu, Y. Fast Colorimetric Sensing of Adenosine and Cocaine Based on a General Sensor Design Involving Aptamers and Nanoparticles. Angewandte Chemie 45, 90-94 (2006).
[16] Zhao, W., Schafer, S., Choi, J., Yamanaka, Y. J., Lombardi, M. G., Bose, S., Carlson, A. L., Phillips, J. H., Teo, W., Droujinine, I. A., Cui, C. H., Jain, R. K., Lammerding, J., Love, J., Lin, C., Sarkar, D., Karnik, R., & Karp, J. M. Cell-surface sensors for real-time probing of cellular environments. Nature Nanotechnology 6, 524-531 (2011).
[17] McNamara, J. A., Andrechek, E. R., Wang, Y., Viles, K. D., Rempel, R. E., Gilboa, E., Sullenger, B. A., & Giangrande, P. H. Cell type-specific delivery of siRNAs with aptamer-siRNA chimeras. Nature Biotechnology 24, 1005-1015 (2006).
[18] Gotrik, M. R., Feagin, T. A., Csordas, A. T., Nakamoto, M., & Soh, H. T . . . . Advancements in Aptamer Discovery Technologies. Accounts of Chemical Research 49, 1903-1910 (2016).
[19] Vant-Hull, B. P., Payano-Baez, A., Davis, R. H., & Gold, L. The mathematics of SELEX against complex targets. Journal of Molecular Biology 278, 579-597 (1998).
[20] Djordjevic, M. SELEX experiments: New prospects, applications and data analysis in inferring regulatory pathways. Biomolecular Engineering 24, 179-189 (2007).
[21] Mendonsa, S. D., & Bowser, M. T. In Vitro Selection of Aptamers with Affinity for Neuropeptide Y Using Capillary Electrophoresis. Journal of the American Chemical Society 127, 9382-9383 (2005).
[22] Hoon, S., Zhou, B., Janda, K. D., Brenner, S., & Scolnick, J. Aptamer selection by high-throughput sequencing and informatic analysis. BioTechniques 51, 413-416 (2011).
[23] Lou, X., Qian, J., Xiao, Y., Viel, L., Gerdon, A. E., Lagally, E. T., Atzberger, P. J., Tarasow, T. M., Heeger, A. J., & Soh, H. T . . . . Micromagnetic selection of aptamers in microfluidic channels. Proceedings of the National Academy of Sciences of the United States of America 106, 2989-2994 (2009).
[24] Kim, J., Olsen, T. W., Zhu, J., Hilton, J., Yang, K., Pei, R., Stojanovic, M., & Lin, Q. Integrated Microfluidic Isolation of Aptamers Using Electrophoretic Oligonucleotide Manipulation. Scientific Reports 6, 26139 (2016).
[25] Wilson, B., & Soh, H. T . . . . Re-Evaluating the Conventional Wisdom about Binding Assays. Trends in Biochemical Sciences 45, 639-649 (2020).
[26] Sun, Y., Gombotz, W. R., & Hoffman, A. Synthesis and Characterization of Non-Fouling Polymer Surfaces: I. Radiation Grafting of Hydroxyethyl Methacrylate and Polyethylene Glycol Methacrylate onto Silastic Film. Journal of Bioactive and Compatible Polymers 1, 316-334 (1986).
[27] Ma, H., Hyun, J., Stiller, P., & Chilkoti, A. “Non-Fouling” Oligo (ethylene glycol)-Functionalized Polymer Brushes Synthesized by Surface-Initiated Atom Transfer Radical Polymerization. Advanced Materials 16, 338-341 (2004).
[28] Sanchez-Cano, C., & Carril, M. Recent Developments in the Design of Non-Biofouling Coatings for Nanoparticles and Surfaces. International Journal of Molecular Sciences 21, 1007 (2020).
[29] MacDonald, J., Houghton, P., Xiang, D., Duan, W., & Shigdar, S. Truncation and Mutation of a Transferrin Receptor Aptamer Enhances Binding Affinity. Nucleic Acid Therapeutics 26, 348-354 (2016).
[30] Le, T. T., Chumphukam, O., & Cass, A. Determination of minimal sequence for binding of an aptamer. A comparison of truncation and hybridization inhibition methods. RSC Advances 4, 47227-47233 (2014).
[31] Bock, L. C., Griffin, L. L., Latham, J., Vermaas, E., & Toole, J. M. Selection of single-stranded DNA molecules that bind and inhibit human thrombin. Nature 355, 564-566 (1992).
[32] Tokmakov, A. A., Kurotani, A., & Sato, K. Protein pI and Intracellular Localization. Frontiers in Molecular Biosciences 8, 775736 (2021).
[33] Ma, X., Balasubramanian, G., & Shrotriya, P. Electrical Stimulus Controlled Binding/Unbinding of Human Thrombin-Aptamer Complex. Scientific Reports 6, 37449 (2016).
[34] Jiang, Z., You, L., Dou, W., Sun, T., & Xu, P. (2019). Effects of an Electric Field on the Conformational Transition of the Protein: A Molecular Dynamics Simulation Study. Polymers 11, 282 (2019).
[35] Thormann, W., Zhang, C., Caslavska, J., Gebauer, P., & Mosher, R. A . . . . Modeling of the Impact of Ionic Strength on the Electroosmotic Flow in Capillary Electrophoresis with Uniform and Discontinuous Buffer Systems. Analytical Chemistry 70, 549-562 (1998).
[36] Acinas, S. G., Sarma-Rupavtarm, R. B., Klepac-Ceraj, V., & Polz, M. F . . . . PCR-Induced Sequence Artifacts and Bias: Insights from Comparison of Two 16S rRNA Clone Libraries Constructed from the Same Sample. Applied and Environmental Microbiology 71, 8966-8969 (2005).
[37] Takahashi, M., Wu, X., Ho, M., Chomchan, P., Rossi, J. J., Burnett, J. C., & Zhou, J. High throughput sequencing analysis of RNA libraries reveals the influences of initial library and PCR methods on SELEX efficiency. Scientific Reports 6, 33697 (2016).
[38] Cho, M., Xiao, Y., Nie, J., Stewart, R., Csordas, A. T., Oh, S. S., Thomson, J. A., & Soh, H. T. Quantitative selection of DNA aptamers through microfluidic selection and high-throughput sequencing. Proceedings of the National Academy of Sciences of the United States of America 107, 15373-15378 (2010).
[39] Kramer, S. T., Gruenke, P. R., Alam, K. K., Xu, D., & Burke, D. H . . . . FASTAptameR 2.0: A web tool for combinatorial sequence selections. Molecular Therapy. Nucleic Acids 29, 862-870 (2022).
[40] Hwang, Y., Sangaj, N., & Varghese, S. Interconnected Macroporous Poly(Ethylene Glycol) Cryogels as a Cell Scaffold for Cartilage Tissue Engineering. Tissue Engineering Part A 16, 3033-3041 (2010).
[41] Thiel, W. H., & Giangrande, P. H. Analyzing HT-SELEX data with the Galaxy Project tools—A web based bioinformatics platform for biomedical research. Methods 97, 3-10 (2016).
[42] CORPET, F. Multiple sequence alignment with hierarchical clustering. Nucleic Acids Research 16, 10881-10890 (1988)
[43] Madeira, F., Pearce, M., Tivey, A R N, et al. Search and sequence analysis tools services from EMBL-EBI in 2022. Nucleic Acids Research 50, 276-279 (2022).
[44] Zuker, M. Mfold web server for nucleic acid folding and hybridization prediction. Nucleic Acids Research 31, 3406-3415 (2003).
[45] Kalra, P., Dhiman, A., Cho, W. C., Bruno, J. F., & Sharma, T. Simple Methods and Rational Design for Enhancing Aptamer Sensitivity and Specificity. Frontiers in Molecular Biosciences, 5, 41 (2018).

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

1. An aptamer-target complex comprising:
an aptamer; and
a target,
wherein the target is conjugated to a porous hydrogel, and
wherein the aptamer selectively associates with the target.

2. The complex of claim 1, wherein the porous hydrogel comprises a non-fouling polymer.

3. The complex of claim 1, wherein the porous hydrogel comprises a cross-linked hydrophilic polymer, a zwitterionic polymer, or mixtures or co-copolymers thereof.

4. The complex of claim 1, wherein the target is selected from a protein, a peptide, a receptor, an antibody, a growth factor, small molecule, toxins, polysaccharides, a cell, a bacteria, and a virus.

5. The complex of claim 4, wherein the target is selected from the group consisting of IL-10, IL-12, and thrombin.

6. The complex of claim 1, wherein the aptamer is a single-stranded nucleic acid.

7. The complex of claim 1, wherein the aptamer comprises DNA, or RNA, or both DNA and RNA.

8. The complex of claim 1, wherein the aptamer comprises at least one chemical modification.

9. The complex of claim 1, wherein the aptamer comprises a detectable moiety.

10. The complex of claim 9, wherein the detectable moiety is selected from the group consisting of a dye, a quantum dot, a radiolabel, an electrochemical functional group, an enzyme, an enzyme substrate, a ligand and a receptor.

11. The complex of claim 1, wherein the aptamer exhibits an affinity constant (Kₐ) of greater than about 10⁵ M⁻¹ with the target.

12. The complex of claim 3, wherein the hydrophilic polymer is selected from the group comprising poly(ethylene glycol), polyoxazoline, polyaliphatic polyurethanes, polyether polyurethanes, polyester polyurethanes, polyethylene copolymers, polyamides, polyvinyl alcohols, poly(ethylene oxide), polypropylene oxide, polypropylene glycol, polytetramethylene oxide, polyvinyl pyrrolidone, polyacrylamide, poly(hydroxy ethyl acrylate), poly(hydroxy ethyl methacrylate), or mixtures or co-polymers thereof.

13. The complex of claim 3, wherein the hydrophilic polymer is poly(ethylene glycol) (PEG) comprising PEG-vinyl sulfone (PEG-VS), PEG-acrylate (PEG-Acr), PEG diacrylate, or mixtures or copolymers thereof.

14. The complex of claim 1, wherein the porous hydrogel has pore sizes of from 1 nm to 200 μm.

15. The complex of claim 1, wherein the porous hydrogel is a macroporous hydrogel.

16. The complex of claim 15, wherein the macroporous hydrogel has a porosity of from 30% to 90%.

17. A method for identifying or producing an aptamer to a target, the method comprising:
contacting a candidate mixture of nucleic acids with the target, wherein nucleic acids having affinity to the target bind the target and form nucleic acid-target complexes;
partitioning nucleic acid-target complexes from free nucleic acids in the candidate mixture; and
identifying nucleic acids that bind to the target, thereby identifying or producing an aptamer to a target;
wherein the target is conjugated to a porous hydrogel.

18. The method of claim 17, further comprising amplifying the nucleic acids that bind to the target.

19.-30. (canceled)

31. The method of claim 17, wherein the porous hydrogel is a macroporous hydrogel having a porosity of from 30% to 90%.

32.-34. (canceled)

35. The method of claim 17, wherein the aptamer exhibits an affinity constant (Ka) of greater than about 105 M-1 with the target.
